(19) United States
(12) Patent Application Publication
(10) Pub. No.: US 2020/0085990 A1
(43) Pub. Date: Mar. 19, 2020

(54) CHEMILUMINESCENT WETNESS INDICATOR FOR ABSORBENT PRODUCTS

(71) Applicant: International Paper Company, Memphis, TN (US)

(72) Inventors: Johnway Gao, Federal Way, WA (US); Brent A. Petersen, Seattle, WA (US); Denise Martin, Issaquah, WA (US); Gary Nicklett, Federal Way, WA (US)

(73) Assignee: International Paper Company, Memphis, TN (US)

(21) Appl. No.: 16/457,732

(22) Filed: Jun. 28, 2019

Related U.S. Application Data
(60) Provisional application No. 62/753,024, filed on Oct. 30, 2018.
(60) Provisional application No. 62/692,502, filed on Jun. 29, 2018.

(51) Int. Cl. A61L 15/38; G01N 21/76; A61L 15/28; A61F 13/42

(52) U.S. Cl. CPC A61L 15/38; A61F 13/42; A61L 15/28; G01N 21/76

(57) ABSTRACT

Disclosed herein are materials and structural elements for absorbent articles that incorporate at least one component of a chemiluminescent system configured to produce light upon contact with an aqueous system. This disclosure also relates to absorbent articles that incorporate materials or structural elements. This disclosure also relates to formulations and methods for treating materials or structural elements with one or more components of such a chemiluminescent system. Representative absorbent articles include disposable diapers and adult incontinence products. Representative chemiluminescent systems include bioluminescent systems.

### CROSS-REFERENCES TO RELATED APPLICATIONS

This application claims the benefit of U.S. Provisional Application No. 62/753,024, filed Oct. 30, 2018, and U.S. Provisional Application No. 62/692,502, filed Jun. 29, 2018, both of which applications are expressly incorporated herein by reference in their entirety.

### TECHNICAL FIELD

Some chemiluminescent systems react in the presence of an aqueous system to produce light. Some of such chemiluminescent systems include components that react in the presence of an aqueous system to produce light, such as a bioluminescent system that includes a luciferin and a luciferase. This disclosure relates to materials, or structural elements for absorbent articles, which are treated with or otherwise integrate at least one component of such a chemiluminescent system. This disclosure also relates to absorbent articles that incorporate such chemiluminescent systems, for example in the aforementioned materials or structural elements. This disclosure also relates to formulations and methods for treating materials or structural elements with one or more components of such a chemiluminescent system.

### BACKGROUND

Personal care absorbent products, such as infant diapers, adult incontinence pads, and feminine care products, as well as related absorbent articles that generally are not worn, such as absorbent bed pads, absorbent pet pads, and the like, typically contain a fluid absorbent core that includes one or more absorbent materials. Although there are many configurations, many absorbent articles include a fluid absorbent core disposed between a top sheet and a back sheet. The top sheet is typically formed from a fluid-permeable material adapted to promote fluid transfer into the absorbent core, such as upon a liquid insult, usually with minimal fluid retention by the top sheet. One absorbent material commonly used in an absorbent core is U.S. southern pine fluff pulp, generally in the form of a fibrous matrix, and sometimes in conjunction with a superabsorbent polymer (SAP) dispersed throughout the fibrous matrix. This fluff pulp is recognized worldwide as a preferred fiber for absorbent products, based on factors such as the fluff pulp's high fiber length, fiber coarseness, and its relative ease of processing from a wet-laid and dried pulp sheet to an air-laid web. The raw material for this type of cellulosic fluff pulp is Southern Pine (e.g., Loblolly Pine, Pinus taeda L.). The raw material is renewable, and the pulp is easily biodegradable. Compared to SAP, these fibers are inexpensive on a per mass basis but tend to be more expensive on per unit of liquid held basis. These fluff pulp fibers mostly absorb within the interstices between fibers. For this reason, a fibrous matrix readily releases acquired liquid on application of pressure. The tendency to release acquired liquid can result in significant skin wetness during use of an absorbent product that includes a core formed exclusively from cellulosic fibers. Such products also tend to leak the acquired liquid because liquid is not effectively retained in such a fibrous absorbent core.

SAPs are water-swellable, generally water-insoluble absorbent materials having a high absorbent capacity for fluids. They are used in absorbent articles like baby diapers or adult incontinent products to absorb and hold body fluids. SAP, upon absorption of fluids, swells and becomes a gel holding more than its weight of such fluids. SAPs in common use are mostly derived from acrylic acid. Acrylic acid based polymers also comprise a meaningful portion of the cost structure of diapers and incontinence pads. SAPs are designed to have high gel strength (as demonstrated by high absorbency under load or AUL). The high gel strength (upon swelling) of currently used SAP particles helps them to retain significant void space between particles, which is helpful for rapid fluid uptake. However, this high “void volume” simultaneously results in significant interstitial (between particles) liquid in the product in the saturated state. When there is interstitial liquid the “rewet” value or “wet feeling” of an absorbent product is compromised.

Advances in SAP technology have allowed the design of absorbent core configurations in which fluff pulp contributes less to the absorbent capability of the core and more to providing a matrix structure in which the SAP is stably held. Fluff pulp fibers also provide fluid distribution functionality, to direct fluid to the SAP. However, it has been found that these structural and fluid distribution functions may be provided, in some configurations, by synthetic fibers, leading to the development of absorbent cores containing both fluff pulp fibers and synthetic fibers, and even “fluff-less” absorbent cores containing no fluff pulp fibers. These configurations may offer the advantage of being less physically bulky, without sacrificing absorbency.

Whatever the configuration, the absorbent core is an absorbent structure that includes one or more materials adapted to absorb a fluid insult. In some configurations, the absorbent core is a self-contained component that is placed into an absorbent article during production. In such configurations, the absorbent materials of the absorbent core (e.g., fluff pulp, synthetic fibers, SAP, and so forth) may be enveloped or at least partially encompassed in a fluid-permeable material, such as a tissue sheet.

Some absorbent articles, such as diapers or adult incontinence pads, also include an acquisition and distribution layer (ADL) for the collection, as well as uniform and timely distribution of fluid from a fluid insult to the absorbent core. An ADL is usually placed between the top sheet and the absorbent core, and normally takes the form of a composite fabric. In an example configuration, the top one-third of such a fabric has low density (higher denier fiber) with relatively large voids and higher void volume for the effective acquisition of the presented fluid, even at relatively higher discharge rates. The middle one-third of the composite fabric of the ADL is usually made of higher density (low denier) fibers with smaller voids, while the lower one-third of the fabric is made of even higher density (lower and smaller denier) fibers and yet with finer voids. The higher density portions of the composite fabric have more and finer capillaries and hence develop greater capillary pressure, thus moving greater volumes of fluid to the outer regions of the structure, thus enabling the proper channelization and distribution of fluid in an even fashion, to allow the absorbent core to take up all of the liquid insult in a time bound manner to allow SAP within the absorbent core to hold and to gel the insult neither too slow nor too fast. The ADL provides for more rapid liquid acquisition (minimizing flooding in the target zone), and ensures more rapid transport and thorough distribution of the fluid into the absorbent core.

As noted above, whatever the configuration, the absorbent core functions to retain fluid, and as such may consist of one or more layers, such as layers to acquire, distribute, and/or store fluid. In many cases, a matrix of cellulose fibers, such as in the form of an air-laid pad and/or non-woven web, is used in (or as) the absorbent core of absorbent articles. In some cases, the different layers may consist of one or more different types of cellulose fibers, such as cross-linked cellulose fibers. In some cases, synthetic fibers, with or without cellulose fibers, may be used. The absorbent core may also include one or more fluid retention agents or other absorbent materials, such as one or more SAPs, distributed throughout the fiber matrix, usually as particles.

The back sheet is typically formed from a fluid-impermeable material to form a barrier to prevent retained fluid from escaping.

Whatever the structure, when the absorbent article is wet from one or more liquid insults, the chances for the fluid coming in contact with the skin increases profoundly, and if left unchanged for a long time can result in diaper rash for infants or dermatitis problems in adults, thereby posing a skin wellness hazard. However, in general, the only way to know whether the absorbent article is dry or wet is to physically inspect it. During day time this may not pose a significant problem, because a caregiver can check worn articles such as diapers or adult incontinent products, or other articles such as a bed pad, as many times as desired. On the other hand, inspections during night time can be a discomfort to the baby as well as to the adult, disturbing their sleep. Moreover, frequent night time inspections, such as several times in a single night, can disrupt the wearer's sleeping pattern, which poses a health hazard to an infant as well as an adult user. In addition, with worn articles such as diapers or incontinence pads, it is typical that an article of clothing, such as pants, pajamas, and/or undergarments, is worn over the absorbent article. With items such as bed pads or pet pads, the position of the user on the pad (human or animal) can obstruct a caregiver's view of it. Accordingly, even absorbent articles that incorporate different types of wetness and/or moisture indicators pose difficulties in timely discovery of an insult.

As a result, there is typically a time lapse between the insult and its discovery. If this time period is prolonged then there exists the possibility of developing diaper rash, skin irritation, and/or skin flaking. These conditions can be very painful for those affected. This is particularly true for babies and those adults in care-giving facilities, and especially true for night time insults, which can lead to longer periods prior to changing the absorbent article.

Although these concerns may not be as immediate for absorbent products that are generally not worn against the body, such as an absorbent bed pad, maintaining user hygiene and comfort is still an important goal.

Previous moisture indicators incorporated into absorbent articles use color change as a visual indication of wetness detection. Inks that appear, or disappear, based on contact with liquid are popular mechanisms for wetness detection. Fluorescence has also been used for wetness detection, such as by incorporating a compound that fluoresces in the presence of a liquid. The mechanisms for such indicators generally fall into three broad categories: (1) imprinting a moisture indicating pattern on one of the plies of the absorbent article; (2) discrete moisture-indicating strips or layers that are incorporated between the layers of the absorbent article; and (3) a discrete (i.e., not part of the absorbent article's construction) indicating strip that is fastened to the interior of the absorbent article immediately prior to use.

Whatever the mechanism, these visual indicators are all deficient in low-light (e.g., night time) situations. Appearing or disappearing inks must be directly visually detected, requiring the caregiver to directly observe the absorbent product. In low-light situations, this may require both a light source (e.g., overhead light or flashlight), as well as the removal of covering garments (e.g., pajamas or undergarments). Fluorescent indicators suffer similar issues, in that they require an external light source to excite the fluorescent compound. Such excitation is typically provided by exposing the indicator to UV light (which presents health concerns to the wearer and caregiver) and must be in direct optical communication with the fluorescent compound, which then requires removal of covering garments, blankets, etc. Therefore, the use of visual indicators previously used to detect wetness in absorbent garments suffers many disadvantages in low-light situations, which greatly reduces the usefulness of their indication mechanisms.

Each of these solutions to wetness detection for absorbent articles is deficient for the needs of night insult detection. Chiefly, all technologies do not reliably trigger, and even when they do, require direct, lighted visual inspection to detect.

Therefore, traditional absorbent articles are inadequate when alerting a caregiver to insults occurring at night and/or under garments.

U.S. patent application Ser. No. 14/516,255, the complete disclosure of which is incorporated herein by reference, discloses fluff pulp compositions treated with a chemiluminescent system configured to produce visible light upon contact with an aqueous system, and absorbent articles that incorporate such treated fluff pulp compositions.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect of this disclosure, materials treated with one or more reactive components of a chemiluminescent system are provided. Generally, two-component systems are discussed, such as bioluminescent systems, which include a luciferin and a luciferase. In such embodiments, the treated material includes at least one reactive component of such a chemiluminescent system. However, these components react in the presence of an aqueous system to produce light. A challenge, therefore, is incorporating the reactive components into a material and/or an absorbent article in a manner that does not prematurely initiate the light-producing reaction, such as during production or storage, but only during use of an absorbent article—specifically, when the absorbent article receives a fluid insult.

Accordingly, some materials are treated with only one reactive component. In use, embodiments in which the treated material includes only one reactive component would typically be incorporated into an absorbent article together with the other reactive component disposed elsewhere in the absorbent article—such as in another treated material or layer—in an arrangement in which one reactive component would be transferred to the other upon receipt of a fluid insult by the absorbent article, such as when the aqueous fluid of the insult moves from the top sheet toward the back sheet (for example), to thereby react and initiate the light-producing reaction. However, in some embodiments, a treated material includes two components that react with each other to produce light. In use, the reaction would typically be initiated when an aqueous system contacts the treated material.

The materials are those that are typically incorporated into an absorbent article. The materials may be absorbent or non-absorbent materials.

In some embodiments, the treated material is a treated tissue composition, in which a liquid permeable tissue sheet is treated on at least one surface with one or both of a luciferin and a luciferase, in a manner in which the component is retained on the surface. The treated tissue composition may be incorporated into an absorbent article, for example as a material in which an absorbent core is enveloped. In some embodiments, the material is an indicator particle formed of hydrogen-bonded cellulose pulp fibers, which is treated with one or more of a luciferin and a luciferase, retained on the fibers on the particle surface and/or throughout the particle. In such embodiments, the indicator particle may take a variety of physical forms. For example, the indicator particle may be in the form of a flake, having two opposed faces and an aspect ratio (the ratio of the length to the width) less than 1.5, with the area of one or both surfaces measuring from 0.1-300 mm². As another example, the indicator particle may be in the form of an elongate strip, having an aspect ratio greater than or equal to 1.5, with a cross-sectional area measuring from 0.01-200 mm², and a length of 1-800 mm. In use, one or more indicator particles may be incorporated into an absorbent article, such as placed or distributed within the absorbent core, or between the absorbent core and the back sheet. The physical form of the particles may vary for a particular configuration and/or size of absorbent core or absorbent article.

In another aspect of this disclosure, an article includes synthetic fibers and at least one of a luciferin and a luciferase. In some embodiments, the synthetic fibers form a nonwoven, absorbent matrix, and thus the article may be suitable for use in, or as, an absorbent core (such as a fluff-less absorbent core) for an absorbent article.

In another aspect of this disclosure, absorbent articles that include a chemiluminescent system, which is configured to produce visible light upon contact with an aqueous system, are provided. In some embodiments, an absorbent article includes a liquid permeable top sheet, a liquid-impermeable back sheet, an absorbent material disposed therebetween, and a chemiluminescent system, with reactive components of the chemiluminescent system separately disposed within the absorbent article in a configuration in which one reactive component is transferred to another by an aqueous system moving through the absorbent article. In such embodiments, the reactive components are disposed in, or on, different structural elements of the absorbent article, such as the absorbent material, the top sheet, the back sheet, a liquid permeable tissue sheet, a treated indicator particle, and so forth. In an illustrative and non-limiting example of such an embodiment, the chemiluminescent system includes a luciferin and a luciferase, with the luciferase disposed within a fibrous absorbent material, and with the luciferin disposed on a tissue sheet, which may form a wrap for the absorbent material such that the absorbent material and tissue sheet together form an absorbent core. In this example, when the absorbent article receives a fluid insult, the fluid moving through the absorbent article toward the absorbent core would encounter the luciferin on the tissue sheet and transfer it to the luciferase (and/or vice versa) in the absorbent material, to thereby initiate the light-producing reaction. In some embodiments that incorporate a fluff-less absorbent core, the chemiluminescent system includes a luciferin and a luciferase, with the luciferase disposed within an absorbent material containing super absorbent materials and with the luciferin disposed on a tissue sheet, which may form a wrap for the absorbent material. In some embodiments incorporating a fluff-less absorbent core, the chemiluminescent system is disposed within the core materials or other structure(s) without a tissue wrapping. In some embodiments, a structural element for incorporation into an absorbent article includes a first surface having a treated area, the treated area being treated with at least one component of a chemiluminescent system, in which the total treated area is less than the area of the first surface.

In yet another aspect of this disclosure, formulations for treating substrate materials with one or more reactive components of a chemiluminescent system—in particular, a luciferin and a luciferase—are provided. In some embodiments, a formulation includes the at least one reactive component (e.g., a luciferin), and a liquid carrier (which may include, e.g., a solvent in which the luciferin is dissolved). Some of such embodiments include a binder adapted to retain the reactive component(s) on the substrate material. Some of such embodiments include a viscosity adjusting agent to impart a desired viscosity to the formulation, for example a viscosity suitable for an application process such as streaming, printing, coating, and so forth. Some of such embodiments include a porous transfer agent. Upon contact of a substrate material treated with a formulation by an aqueous system, the porous transfer agent is adapted to facilitate transfer of the reactive component(s), and/or the aqueous system, relative to the substrate material. In other embodiments, at least one component of a chemiluminescent system is applied to one or more substrates as a dry formulation within a dry carrier material. Dry carrier materials may include any inert material to allow for flowable or dispersible formulations within application machinery, such as, but not limited to, sugars, minerals or salts thereof, starch, silica, clays, talc, micronized wood or other cellulose, gelatins, agars, and SAP.

In some embodiments, a formulation includes both a luciferin and a luciferase, and the liquid carrier is, or includes, a solvent. In such embodiments, the luciferin is dissolved in the solvent, and the luciferase is dispersed in the liquid carrier.

In some embodiments particularly for applying a luciferin to a substrate material, a partially aqueous formulation is used. Such embodiments include a luciferin, a solvent to dissolve the luciferin that includes water, and an excipient (e.g., hydroxypropyl-β-cyclodextrin, ethanol, water soluble polymers such as poly(ethylene glycol), poly(vinyl alcohol), partially hydrolyzed poly(vinyl alcohol), polyvinylpyrrolidone, poly(1-vinylpyrrolidone-co-2-dimethylaminoethylmethacrylate), poly(l-vinylpyrrolidone-co-vinyl acetate), and combinations thereof; sugars (mono-saccharides, poly-saccharides, and branched polysaccharides); cellulose and cellulose derivatives; minerals and salts thereof; etc.) adapted to facilitate the solubility of the luciferin in water. Some of such embodiments include a binder adapted to bind the luciferin to the substrate material. Some excipients, while inert in the chemiluminescent reaction, provide additional benefits. Such additional benefits include, for example, effects on solubility of a given component of the chemiluminescent system in various aqueous and non-aqueous solvent systems, effects on water availability in an absorbent article, retention effects (e.g., binders) on various substrates for one or both components of the chemiluminescent system, releasing effects (e.g., porous transfer agents) for one or both components of the chemiluminescent system, etc. Some such excipients may have multiple of these or other functions. Thus, the excipient use in the present teaching is not limited to partially aqueous formulations.

Within these broad parameters, formulations in accordance with this disclosure may include components, and relative amounts thereof, suitable for a broad variety of applications. In an illustrative and non-limiting example of such an embodiment, a formulation suitable for applying coelenterazine (a luciferin) to a liquid-impermeable back sheet, which are typically formed from synthetic materials, includes ethanol, coelenterazine, a binder, and a porous transfer agent. In another illustrative and non-limiting example, a formulation suitable for applying coelenterazine and a luciferase to a cellulosic substrate material includes ethanol, coelenterazine, a luciferase (such as Gaussia, Renilla, and/or Metridia luciferase), a binder and/or a viscosity adjusting agent, and a porous transfer agent. As explained in greater detail herein, the nature of the substrate material to which a formulation is applied is a factor for determining whether a binder is suitable. For example, a binder may benefit a formulation to be applied to a substrate material that includes synthetic fibers, whereas a binder may not be needed in a formulation to be applied to a substrate material made up of cellulose fibers.

In yet another aspect of this disclosure, methods for treating a substrate material with one or more reactive components of a chemiluminescent system are provided. As noted above, these components react in the presence of an aqueous system to produce light. Accordingly, one challenge in incorporating the reactive components into a material and/or an absorbent article is doing so in a manner that does not prematurely initiate the light-producing reaction. In some embodiments, a method includes applying a formulation of a luciferase dispersed in an aqueous liquid to a substrate material sufficient to achieve a desired luciferase concentration on the substrate material, but without raising the moisture content of the substrate material above a threshold level of moisture. Such a method may offer the advantage of reducing the need for a subsequent drying step, such as if a luciferin is also applied to the substrate material. In some embodiments, a method includes separate luciferase and luciferin treatment steps, in which an area on a surface of a substrate material is treated with a luciferase formulation that includes a luciferase dispersed in an aqueous liquid, and separately with a luciferin formulation that includes a luciferin dissolved in a non-aqueous solvent.

In yet another aspect, methods for producing absorbent articles, or structural elements for incorporation into absorbent articles, for example utilizing one or more of the aforementioned treated materials, are provided. In some embodiments, a treated tissue composition may be produced by applying a formulation that includes luciferin dissolved in a solvent to a liquid permeable tissue sheet, for example by streaming the formulation to the surface, and subsequently removing the solvent from the tissue sheet. In some of such embodiments, the tissue sheet may be a continuous sheet that is moved relative to one or more nozzles that stream the formulation, with the surface of the tissue sheet to which the formulation is applied being suspended between two fixed points. In such embodiments, the solvent may be removed by subsequent heat treatment of the treated surface, for example by moving the treated surface through a heated zone.

In yet another aspect, a chemiluminescent system is provided in a form suitable for use in an absorbent article, or in one or more materials and/or structural elements thereof. In some embodiments, a composition includes an encapsulated material consisting of particles comprising a predetermined quantity of a first component of a chemiluminescent system, with the particles having a water-permeable or water-soluble coating that covers the entire surface of the particle, and a predetermined quantity of a second component of the chemiluminescent system. Such a composition may be suitable for incorporation into an absorbent article, or a component thereof, for example during production, or by an end user prior to use of the absorbent article. Some embodiments may be in the form of a kit that includes an absorbent article in which a first component of a chemiluminescent system is incorporated, and a measured quantity of a second component of the chemiluminescent system suitable to react with the first component to produce light of a predetermined duration and/or intensity. In some of such embodiments, the measured quantity may be in the form of a liquid formulation, gel, powder, and so forth, for example for an end user of the absorbent article to apply to the absorbent article prior to its use.

Representative absorbent articles include disposable diapers and adult incontinence products.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 photographically depicts an example absorbent article that incorporates a chemiluminescent fluff pulp composition.

FIG. 2A is a chemical structure of a representative luciferin compound useful in chemiluminescent systems in accordance with embodiments disclosed herein;

FIG. 2B is a chemical structure of a representative luciferin compound useful in chemiluminescent systems in accordance with embodiments disclosed herein;

FIG. 2C is a chemical structure of another representative luciferin compound useful in chemiluminescent systems in accordance with embodiments disclosed herein;

FIG. 2D is a chemical structure of another representative luciferin compound useful in chemiluminescent systems in accordance with embodiments disclosed herein;

FIG. 2E is a chemical structure of another representative luciferin compound useful in chemiluminescent systems in accordance with embodiments disclosed herein;

FIG. 2F is a chemical structure of another representative luciferin compound useful in chemiluminescent systems in accordance with embodiments disclosed herein;

FIG. 2G is a chemical structure of another representative luciferin compound useful in chemiluminescent systems in accordance with embodiments disclosed herein;

FIG. 2H is a chemical structure of another representative luciferin compound useful in chemiluminescent systems in accordance with embodiments disclosed herein;

FIG. 3 graphically illustrates spectral properties of representative luciferins in accordance with embodiments disclosed herein;

FIG. 4A illustrates a perspective view of a non-limiting, representative example of an absorbent article (in the form of a diaper) in accordance with embodiments disclosed herein;

FIG. 4B is a top-down plan view of the absorbent article of FIG. 4A;

FIG. 4C illustrates an example cross-section of the absorbent article of FIG. 4A in accordance with an embodiment of the disclosure;

FIG. 4D illustrates an example cross-section of the absorbent article of FIG. 4A in accordance with an embodiment of the disclosure;

FIG. 4E illustrates an example cross-section of the absorbent article of FIG. 4A in accordance with an embodiment of the disclosure;

FIG. 4F illustrates an example cross-section of the absorbent article of FIG. 4A in accordance with an embodiment of the disclosure;

FIG. 5A illustrates a perspective view of a non-limiting, representative example of a treated tissue composition in accordance with embodiments disclosed herein;

FIG. 5B illustrates a perspective view of a non-limiting, representative example of another treated tissue composition in accordance with embodiments disclosed herein;

FIG. 5C illustrates a perspective view of a non-limiting, representative example of another treated tissue composition in accordance with embodiments disclosed herein;

FIG. 5D illustrates a cross-section view of a non-limiting, representative example of a treated tissue composition in accordance with an embodiment of the disclosure;

FIG. 5E illustrates a cross-section view of a non-limiting, representative example of another treated tissue composition in accordance with an embodiment of the disclosure;

FIG. 5F illustrates a cross-section view of a non-limiting, representative example of another treated tissue composition in accordance with an embodiment of the disclosure;

FIG. 5G illustrates a cross-section view of a non-limiting, representative example of another treated tissue composition in accordance with an embodiment of the disclosure;

FIG. 6A illustrates a non-limiting, representative example of an indicator particle in accordance with embodiments disclosed herein;

FIG. 6B illustrates a non-limiting, representative example of another indicator particle in accordance with embodiments disclosed herein;

FIG. 7 illustrates a non-limiting, representative example of an article in the form of an absorbent core in accordance with embodiments disclosed herein;

FIG. 8A schematically illustrates a non-limiting, representative example of a streaming apparatus suitable for use in producing treated tissue compositions in accordance with embodiments disclosed herein;

FIG. 8B schematically illustrates a non-limiting, representative example of a streaming apparatus suitable for use in producing treated tissue compositions in accordance with embodiments disclosed herein;

FIG. 9A illustrates a non-limiting, representative example of a structural element for incorporation into an absorbent article, in accordance with embodiments disclosed herein;

FIG. 9B illustrates a non-limiting, representative example of another structural element for incorporation into an absorbent article, in accordance with embodiments disclosed herein;

FIG. 9C illustrates a non-limiting, representative example of another structural element for incorporation into an absorbent article, in accordance with embodiments disclosed herein;

FIG. 9D illustrates a non-limiting, representative example of another structural element for incorporation into an absorbent article, in accordance with embodiments disclosed herein;

FIG. 9E illustrates a non-limiting, representative example of another structural element for incorporation into an absorbent article, in accordance with embodiments disclosed herein;

FIG. 9F illustrates a non-limiting, representative example of another structural element for incorporation into an absorbent article, in accordance with embodiments disclosed herein;

FIG. 9G illustrates a non-limiting, representative example of another structural element for incorporation into an absorbent article, in accordance with embodiments disclosed herein;

FIG. 9H illustrates a non-limiting, representative example of another structural element for incorporation into an absorbent article, in accordance with embodiments disclosed herein;

FIG. 9I illustrates a non-limiting, representative example of another structural element for incorporation into an absorbent article, in accordance with embodiments disclosed herein;

FIG. 9J illustrates a non-limiting, representative example of another structural element for incorporation into an absorbent article, in accordance with embodiments disclosed herein;

FIG. 10 graphically illustrates chemiluminescent intensity as a function of time exhibited by a representative example treated material in accordance with embodiments disclosed herein;

FIG. 11 graphically illustrates chemiluminescent intensity as a function of time exhibited by a representative example treated material in accordance with embodiments disclosed herein;

FIG. 12 graphically illustrates chemiluminescent intensity as a function of time exhibited by a representative example treated material in accordance with embodiments disclosed herein;

FIG. 13 graphically illustrates chemiluminescent intensity as a function of time exhibited by a representative example treated material in accordance with embodiments disclosed herein;

FIG. 14A graphically illustrates chemiluminescent intensity as a function of time exhibited by a representative example treated material in accordance with embodiments disclosed herein;

FIG. 14B graphically illustrates chemiluminescent intensity as a function of time exhibited by a representative example treated material in accordance with embodiments disclosed herein;

FIG. 15A graphically illustrates chemiluminescent intensity as a function of time exhibited by a representative example treated material in accordance with embodiments disclosed herein;

FIG. 15B graphically illustrates chemiluminescent intensity as a function of time exhibited by a representative example treated material in accordance with embodiments disclosed herein; and

FIG. 16 graphically illustrates chemiluminescent intensity as a function of time exhibited by a representative example treated material in accordance with embodiments disclosed herein.

### DETAILED DESCRIPTION

While illustrative embodiments have been illustrated and described, it will be appreciated that various changes can be made therein without departing from the spirit and scope of the invention.

Disclosed herein are materials treated with one or more reactive components of a chemiluminescent system, absorbent articles and structural elements for absorbent articles that incorporate these materials, formulations, compositions, and treatment and production methods relating to a chemiluminescent system and its use in absorbent articles. The chemiluminescent system is configured to produce light upon contact with an aqueous system. The reactive components of the chemiluminescent system are generally disposed in one or more treated materials and/or compositions incorporated into the absorbent articles. Representative absorbent articles include disposable diapers and adult incontinence products. Representative chemiluminescent systems include bioluminescent systems.

Chemiluminescence results from a chemical reaction that produces light, and therefore provides a lighted indication of moisture that can be seen in low light and/or in the absence of light, and through clothing. Furthermore, chemiluminescence requires no external excitation light, as is required for photoluminescent (e.g., fluorescent) indicators. Accordingly, by generating light upon contact with an aqueous system (e.g., urine), incorporating a chemiluminescent system greatly enhances the ability of absorbent articles to indicate the occurrence of an insult in darkened conditions (e.g., at night). Moreover, by generating light that can be detected through clothing, a caregiver may be able to ascertain the occurrence of an insult without having to move or disturb the infant or adult wearer of such an absorbent article, such as during sleep. Therefore, the various compositions and articles provided herein may provide the distinct advantages of insult indication at night and through clothing, which may reduce or even eliminate the need for caregivers to disturb the sleep (e.g., by pulling down clothing and/or shining a light) of one wearing such an absorbent article in order to test for an insult. Further, because visible light (that is, light in the visible spectrum) is produced by the chemiluminescent systems disclosed herein, there is no need to expose the absorbent article in which the system is incorporated, and/or the wearer, to UV light in order to determine whether an insult has occurred, allowing health concerns associated with UV radiation to be avoided.

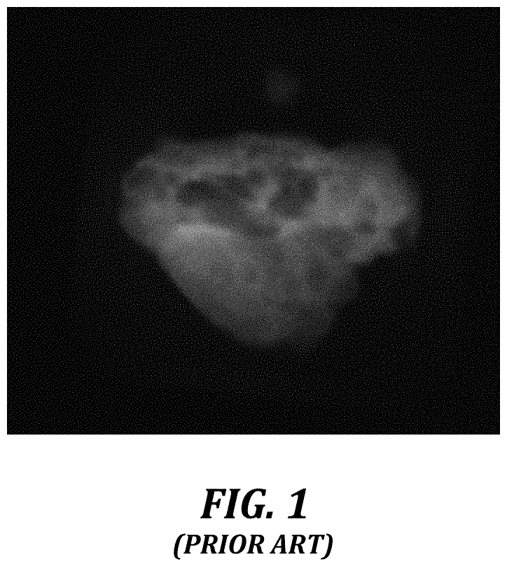

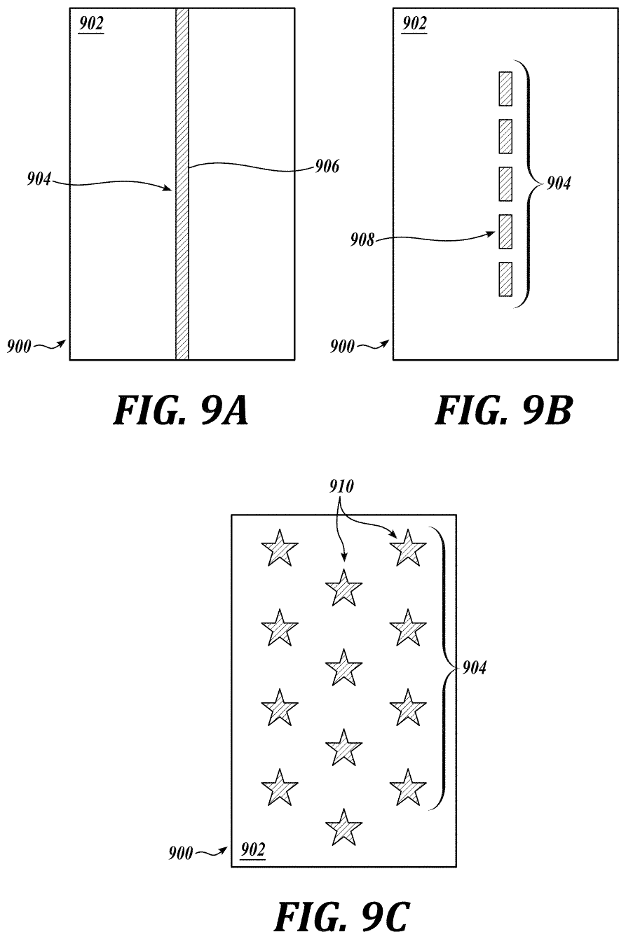

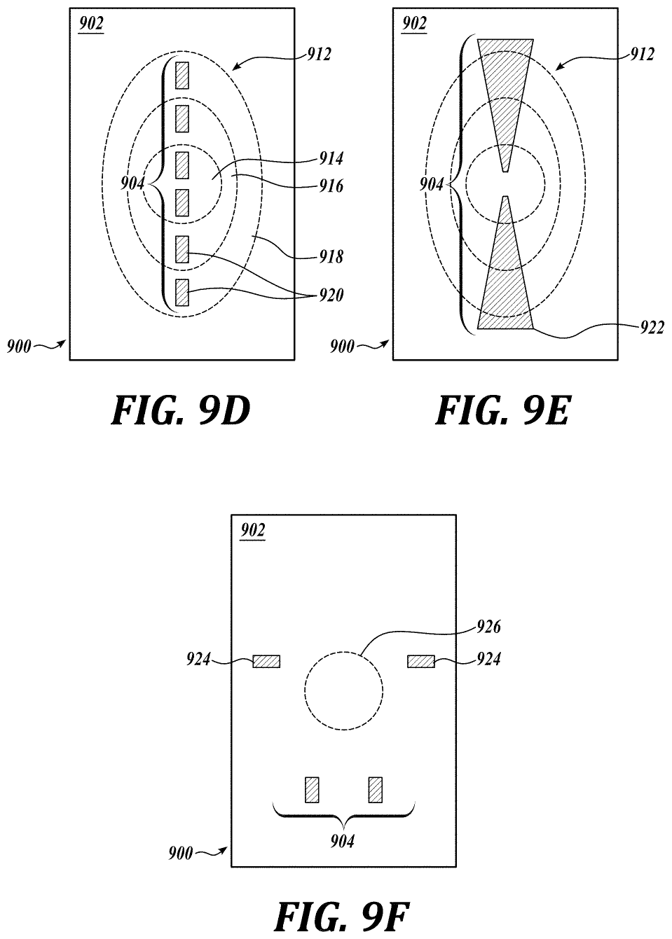

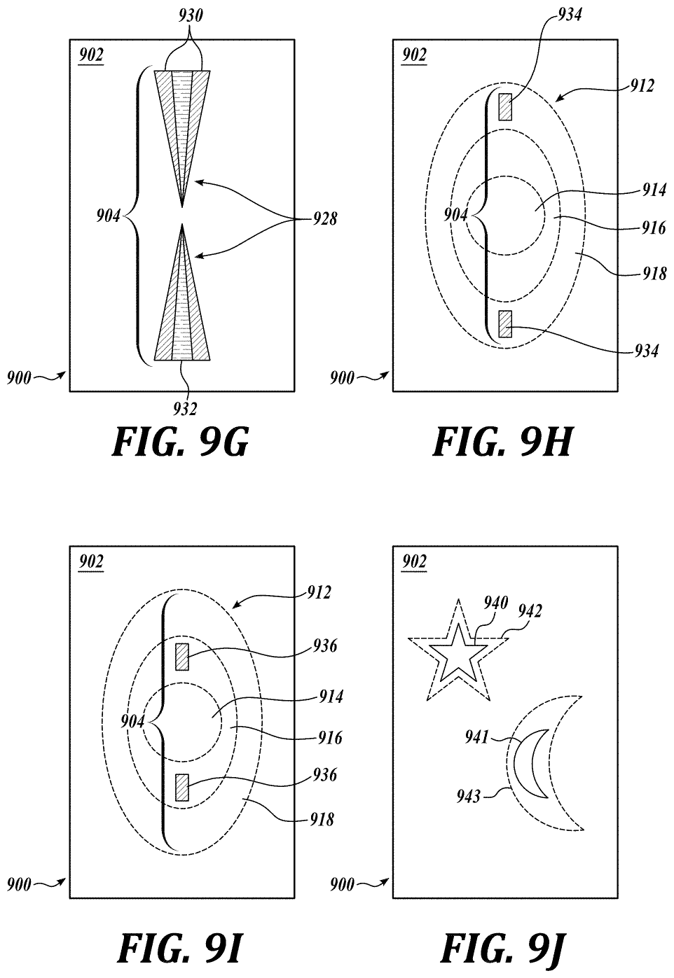

The low-light detection provided by a chemiluminescent system, and in particular certain embodiments of absorbent articles incorporating such a system, is discussed in Applicant's aforementioned co-pending U.S. patent application Ser. No. 14/516,255, and illustrated in some of the drawings therein. For example, FIG. 1, reproduced from the '255 application, is a photograph of an absorbent article (diaper) incorporating an absorbent core that includes a chemiluminescent fluff pulp composition (a fluff pulp treated with both luciferase and luciferin). In FIG. 1, a mock insult (saline solution) was applied and the image was captured showing chemiluminescence shining through the diaper back sheet and light-weight cotton fabric for easy visual detection in low-light conditions. A comparative absorbent article formed using fluorescent instead of chemiluminescent fluff does not function through the diaper material, due to blocked excitation via an external light source (see, e.g., FIG. 9A of the '255 application). Activation of a fluorescent wetness indicator requires removal of clothing, etc., and application of an excitation light (e.g., UV light) in order to visually detect an insult. Chemiluminescence requires neither removal of clothing nor excitation light.

The improved ease with which an insult can be detected with the absorbent articles disclosed herein allows the caregiver to check for an insult as needed (e.g., more frequently), due to the reduced interruption required. More frequent checks may allow an insult to be detected sooner and the absorbent article changed soon after the insult, thereby reducing the amount of time the insult contacts the wearer's skin, as well as reducing the possibility of fluid from multiple insults contacting the wearer's skin. The skin health and general comfort of the wearer are improved when the length of time that fluid is in contact with the skin is reduced.

In one aspect, materials treated with one or more reactive components of a chemiluminescent system, which react in the presence of an aqueous system to produce visible light, are provided. The term “visible light” herein refers to light in the visible spectrum. The materials are those that are typically incorporated into an absorbent article. The materials may be absorbent or non-absorbent materials.

### Chemiluminescent System

The chemiluminescent system includes at least two reactive components configured to react in the presence of an aqueous system to produce visible light. Put another way, the aqueous system initiates the chemiluminescence reaction between the reactive components in order to produce light. The chemiluminescent system is adapted to react in the presence of an aqueous system to produce light. In preferred embodiments, the produced light is visible light that can be observed by a human. In some embodiments, only a single reaction component selected from luciferin and luciferase is present in a given structural element within an article as described herein. In such embodiments, the chemiluminescent system is configured to produce light when in the presence of an aqueous system and the other of the reaction components. In preferred embodiments, the produced light is visible light that can be observed by a human. In some embodiments, the aqueous system functions to transfer one reactive component to the other, to thereby initiate the light-producing reaction. As used herein, the term “aqueous system” refers to water or water-containing compositions. In the context of this disclosure, such water-containing compositions are generally in the form of body fluid, such as urine, menses, fecal matter, and so forth. The occurrence of the release of bodily fluid (or the fluid itself) is referred to herein as an “insult,” “liquid insult,” or “fluid insult.” Accordingly, the chemiluminescent systems of the present disclosure produce light upon insult of an article in which the reactive components of the chemiluminescent system are incorporated.

In being configured to produce visible light upon contact with an aqueous system, one of the reactive components of the chemiluminescent system luminesces when the reactive components react in the presence of an aqueous system. In some embodiments, water is the component of the aqueous system that initiates the light-producing reaction. In these embodiments, the reactive components do not react without the presence of the aqueous system. In these embodiments, the reactive components do not independently luminesce.

Representative chemiluminescent systems that include two or more reactive components include bioluminescent systems, such as a system that includes a luciferin and a luciferase.

Bioluminescence is light that is produced by a chemical reaction that occurs within the body or in the secretions of certain types of organisms. Bioluminescence involves the combination of two types of substances in a light-producing reaction: a luciferin and a luciferase. Luciferin is the compound that actually luminesces—that is, that produces the light. Luciferase is an enzyme that catalyzes the reaction. In some cases luciferase is a protein known as a photoprotein, and the light making process requires a charged ion (e.g., a cation such as calcium) to activate the reaction. Often, the bioluminescence process requires the presence of a substance such as oxygen or adenosine triphosphate (ATP) to initiate the oxidation reaction. The reaction rate for the luciferin is often controlled by the luciferase. The luciferin-luciferase reaction can also create byproducts such as inactive oxyluciferin and water.

Luciferin and luciferase are generic names rather than specific materials. For example, the luciferin coelenterazine (natural form) is common in marine bioluminescence but variants can be chemically synthesized and these various forms are collectively called luciferins. Several synthesis methods for coelenterazine are disclosed in U.S. Provisional Application No. 62/692,485, the entire content of which is incorporated herein by reference.

The mechanism of light production through a chemical reaction differentiates bioluminescence from other optical phenomenon such as fluorescence or phosphorescence.

For example, fluorescent molecules do not emit their own light. They need an external photon source to excite their electrons to a higher energy state. On relaxation from the high energy state to their natural ground state, they release their acquired energy as a light source, but usually at a longer wavelength. Since the excitation and relaxation occurs simultaneously, fluorescent light is seen only when illuminated (excited).

The term phosphorescence technically refers to a special case of optically excited light emission where the relaxation from the excited state to ground state, unlike the fluorescence, is not immediate, and the photon emission persists for seconds to minutes after the original excitation.

The technical distinction between bioluminescence and fluorescence is sometimes blurred in a practical context, but, technically, they are two distinct phenomena. In most cases, a bioluminescent can be an autofluorescent but the reverse is not true for a fluorescent; the latter still requires photon for excitation to emit light. In some cases bioluminescent cnidarians, crustaceans, or fish can contain a fluorescent protein, like Green Fluorescent Protein (GFP), and the light emitted from the bioluminescent would act as photons to excite the GFP. The GFP in turn under relaxed state would emit a light of different wavelength (most probably of higher wavelength) than the wavelength of the bioluminescent light that it has received as a photon. In this example, the GFP may be excited by a blue light emitted by the bioluminescent (wave length 470 nm) but in turn would emit a green light under its relaxed state (wave length of 510 nm to 520 nm).

Bioluminescent systems can be incorporated into absorbent articles in any manner that produces the desired chemiluminescence. Several are disclosed herein.

In some embodiments, the luciferin is selected from the group consisting of coelenterazine, a coelenterazine analog, dinoflagellate luciferin, bacterial luciferin, fungal luciferin, firefly luciferin, vargulin, and furimazine. With regard to coelenterazine, there is a native form as well as many analogs, or variants, any of which can be used in the embodiments and methods disclosed herein. For clarity, the term “a coelenterazine analog” refers to substances such as methyl coelenterazine, coelenterazine 400a, (2-2′(4-dehydroxy)) coelenterazine, coelenterazine e, coelenterazine f, coelenterazine h, coelenterazine i, coelenterazine n, coelenterazine cp, coelenterazine ip, coelenterazine fcp, and coelenterazine hcp, whereas “coelenterazine” refers to native coelenterazine.

Thus, in some embodiments, the coelenterazine may be one or more of native coelenterazine and a coelenterazine analog. As an example, the coelenterazine may be one or more of native coelenterazine, coelenterazine 400a, methyl coelenterazine, coelenterazine f, coelenterazine cp, coelenterazine fcp, and coelenterazine hcp. As yet a further example, the coelenterazine may be one or more of coelenterazine 400a, methyl coelenterazine and coelenterazine fcp. As yet a further example, the coelenterazine may be one or more of coelenterazine 400a, methyl coelenterazine, and coelenterazine hcp. In yet another example, the coelenterazine may be may be one or more of coelenterazine 400a and coelenterazine hcp.

In some embodiments, the luciferase is selected from the group consisting of Gaussia luciferase (GLuc), Renilla luciferase (RLuc), Metridia luciferase, Oplophorus luciferase, dinoflagellate luciferase, firefly luciferase, fungal luciferase, bacterial luciferase, copepod luciferase, and vargula luciferase. Certain embodiments of the luciferase consistent with this disclosure comprise one or more of Gaussia luciferase, Renilla luciferase, dinoflagellate luciferase, and firefly luciferase. As a further example, the luciferase may be one or more of Gaussia luciferase, Renilla luciferase, dinoflagellate luciferase, and firefly luciferase. In yet a further example, the luciferase may be one or more of Gaussia luciferase, Renilla luciferase, and Metridia luciferase.

In some embodiments, the chemiluminescent system comprises coelenterazine as the luciferin and/or Gaussia, Renilla, and Metridia luciferases, or combinations thereof.

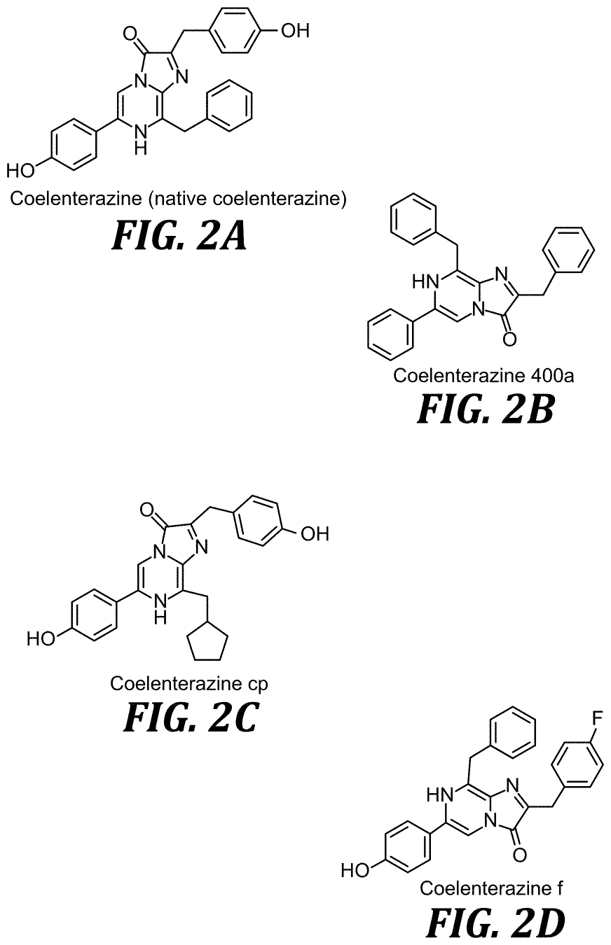

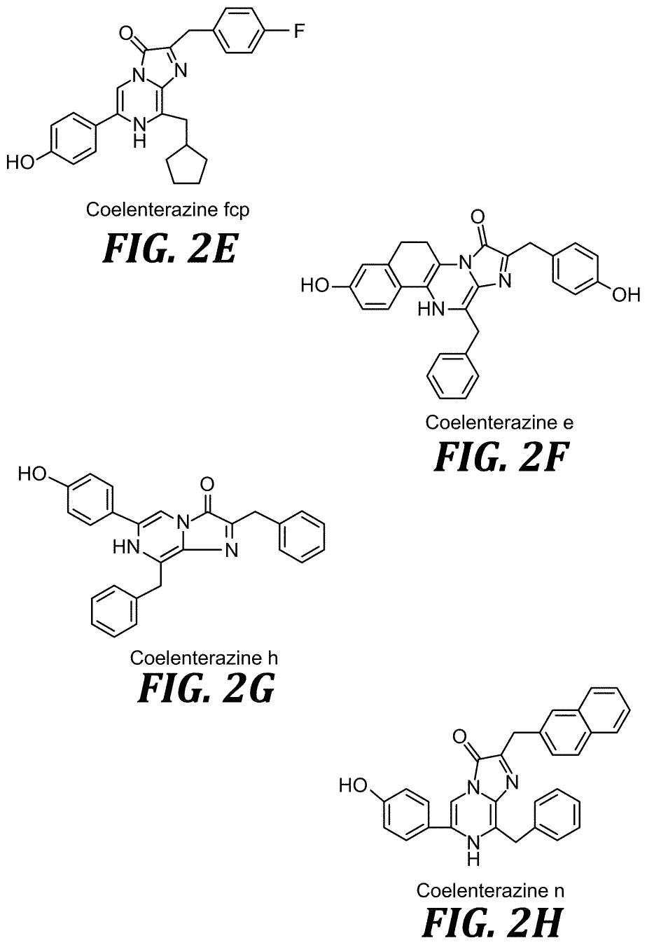

Coelenterazine in its native form and its analogs have different luminescent characteristics due to variation in their structural moieties. Given structural variations within the coelenterazine family, some are good substrates for luciferase, whereas some are not. Below is a brief description of native coelenterazine and representative analogs. Coelenterazine (native form), illustrated in FIG. 2A, is a luminescent enzyme substrate for Renilla (reniformis) luciferase (RLuc). Renilla luciferase/coelenterazine has also been used as the bioluminescence donor in bioluminescence resonance transfer (BRET) studies. Unless otherwise specified, the unmodified term “coelenterazine” refers to coelenterazine in its native form.

Coelenterazine 400a, illustrated in FIG. 2B, is a derivative of coelenterazine and is a good substrate for Renilla luciferase (RLuc), but does not oxidize well with Gaussia luciferase (GLuc). It is the preferred substrate for BRET (bioluminescence resonance energy transfer) because its emission maximum of 400 nm has minimal interference with the GFP emission.

Fluorescence resonance energy transfer (FRET), BRET, resonance energy transfer (RET), and electronic energy transfer (EET) are mechanisms describing energy transfer between two light-sensitive molecules (chromophores), and usually define the interference of a luminescent chemical with the energy transfer of another luminescent chemical, thus decreasing the energy state to which the latter can be taken, which is critical in terms of relaxation energy released back to the ground state. A donor chromophore, initially in its electronic excited state, may transfer energy to an acceptor chromophore through nonradiative dipole-dipole coupling. The efficiency of this energy transfer is inversely proportional to the sixth power of the distance between donor and acceptor, making FRET extremely sensitive to small changes in distance. Measurements of FRET efficiency can be used to determine if two fluorophores are within a certain distance of each other. Such measurements are used as a research tool in fields including biology and chemistry.

For example, BRET in the presence of Renilla luciferase (RLuc) by coelenterazine 400a is compared to coelenterazine (native form) and clearly shows minimal interference with the GFP emission, as illustrated in FIGS. 2A and 2B, wherein “hRluc” is Renilla luciferase and a coelenterazine h bioluminescence system; “FLuc” is Renilla luciferase with a native coelenterazine system; “lucroron” is a luciferase and luciferin system with GFP2 as a photon acceptor that fluoresces resulting from emission from the hRLuc bioluminescence; and “GFP2” is Green Fluorescent Protein (2nd generation).

Coelenterazine cp, illustrated in FIG. 2C, is a coelenterazine-aequorin complex generates luminescence intensity 15 times higher than coelenterazine (native form).

Coelenterazine f, illustrated in FIG. 2D, has 20 times higher luminescence intensity (coelenterazine-apoaequorin complex) than the native form coelenterazine, while its emission maximum is about 8 nm longer than that of the native form.

Coelenterazine fcp, illustrated in FIG. 2E, is an analog wherein the o-benzene structure in the elenterazine moiety of coelenterazine f structure is replaced with a cyclic pentane (similar to coelenterazine cp). Coelenterazine fcp has luminescence intensity 135 times greater than that of coelenterazine (native form).

Coelenterazine fcp complexes with aequorin to form a coelenterazine fcp-apoaequorin complex and, as a substrate for aequorin, has a relative luminescence intensity of 135 times as compared to native form coelenterazine. However, coelenterazine fcp is a poor substrate for Renilla luciferase.

Other representative analogs of coelenterazine, as a substrate for Renilla luciferase enzyme, are coelenterazine e, h and n, illustrated in FIGS. 2F, 2G, and 2H, respectively. While these three analogs are good to excellent substrates for Renilla luciferase, they are poor substrates for apoaequorin.

The luminescent properties of coelenterazine analogs vary. For example, certain analogs emit less light (as measured as lumens) but with higher luminescent intensity (lumens/steradian). Table 1 lists the luminescent properties of coelenterazine (native form) and its analogs with Renilla luciferase. Luminescent intensity is reported as a % initial intensity. For example, an analog having an initial intensity of 900% is 20 times intense as compared to the native coelenterazine with an initial intensity of 45%.

TABLE 1
Luminescent Properties of Selected Coelenterazine Analogs
with Renilla Luciferase

| Analog | λₑₘ (nm) | Total Light (%) | Initial Intensity (%) |
|---|---|---|---|
| native | 475 | 100 | 45 |
| cp | 470 | 23 | 135 |
| e | 418, 475 | 137 | 900 |
| f | 473 | 28 | 45 |
| h | 475 | 41 | 135 |
| n | 475 | 47 | 900 |

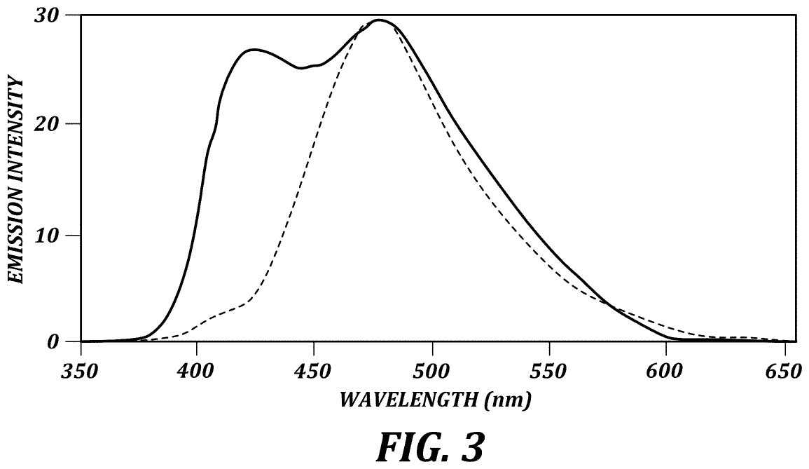

The emission spectra (normalized) of coelenterazine e (with a luminescent intensity 20 times higher than that of native coelenterazine) and native coelenterazine are illustrated in FIG. 3. In FIG. 3, coelenterazine e (solid line) and native coelenterazine (dotted line) are measured in the presence of recombinant Renilla luciferase (RLuc). Note there are two intensity peaks for coelenterazine e, one at a wavelength (λₑₘ) of 418 nm and the other at 475 nm.

Visible light—that is, light in the visible spectrum—is produced by the chemiluminescent systems. The light is visually detectable by a caregiver in the dark and through clothing and/or coverings, and as such has a wavelength, intensity, and duration sufficient to provide the necessary indication. These spectral characteristics of the chemiluminescent systems can be tailored based on the chemiluminescent compound or compounds. For example, in bioluminescent systems, the luciferin and luciferase can be selected to produce the desired light characteristics. Depending on the bioluminescent system used, different spectral characteristics can occur. In the presence of superoxide anions and/or peroxynitrile compounds, coelenterazine can also emit light independent of enzyme (luciferase) oxidation, a process known as autoluminescence.

The chemiluminescent systems can be tailored to produce particular colors of visible light. As noted above in Table 1, even within the coelenterazine family, the emission wavelength can range from about 400 nm (violet) to about 475 nm (blue with green tint).

With regard to duration, the duration of the light emitted may be controlled by the selection of the coelenterazine (luciferin), in native form versus its analogs, and the enzyme (luciferase), for example Gaussia versus Renilla. The ratio and the concentration of luciferin and luciferase used may also modify the duration of light emission. To give an illustrative and non-limiting example, the luciferin analog, coelenterazine e, has a total light of 130% and initial intensity of 900% (see FIG. 3) over native coelenterazine. By judiciously selecting the concentration of coelenterazine e and Renilla luciferase, the duration of the visible light emitted can last as much as 8 to 10 hours.

In some embodiments, the visible light has a duration of 0.5 to 6 hours. In some embodiments, the visible light has a duration of 1 to 4 hours. In some embodiments, the visible light has a duration of 2 to 3 hours. For example, the visible light can have a duration of between 1-2 hours, 1-3 hours, 1-4 hours, 1-5 hours, 1-6 hours, or within any range within any of these ranges, and all other possible subranges.

With regard to intensity, quantum efficiency of the chemiluminescence contributes to the intensity, depth, and hue of the color of the emission.

Quantum efficiency (QE) is the fraction of photon flux used to excite a luminescence chemical to elevate it to higher energy state. Quantum efficiency is one of the most important parameters used to evaluate the quality of a detector and is often called the “spectral response” to reflect its wavelength dependence. It is defined as the number of signal electrons created per incident photon. In some cases it can exceed 100% (i.e., when more than one electron is created per incident photon). If the spectral response exceeds 100%, then the intensity and depth of the color emitted is vivid, but depending on the status of the excited state of the primary electron, the duration of the emission will be determined (i.e., the higher the excited state, the more time it takes to return to the ground (normal) state).

Spectral responsivity is a similar measurement, but it has different units; the metric being the amperes or watts (i.e., how much current comes out of the device per incoming photon of a given energy and wavelength).

Both the quantum efficiency and the spectral responsivity are functions of the photons' wavelength. For example, in the case of the luciferin coelenterazine, between the native form and one of its analogs, coelenterazine e, the latter has not only high light intensity but emits 30% more light energy than the former, because the latter upon excitation by a given quanta (hv) of incident photon generates two electrons and the primary electron at wavelength 475 has the same emission intensity as native coelenterazine but with lumen intensity 20 times greater than that of the native product. Accordingly, the light emitted by the excited coelenterazine analog would be twenty times brighter than the native form but with a total light energy of 130% will last longer than the native form.

The wavelength determines the color of the emitted visible light.

The chemiluminescent systems of the present disclosure usually include two reactive components, such as a luciferin and a luciferase, sometimes referred to herein as “a luciferin component” and “a luciferase component,” respectively. The treated materials in accordance with an aspect of the present disclosure include at least one, and in some embodiments two, of the reactive components. The absorbent articles in accordance with another aspect of the disclosure generally incorporate both of the aforementioned reactive components, such as by including one or more materials that are treated with the reactive components. The term “incorporate,” when referring to the reactive components, indicates that the components are included in the absorbent articles in some manner. In some embodiments, one or more components are retained on the surfaces of, or within, certain materials or structural elements of the absorbent article, such as on the cellulose and/or other fibers of treated materials, for example by means of a binder or otherwise through chemical or mechanical interaction. In some embodiments, one or more components are distributed (such as in granular, powder, or other particulate form) on, or throughout, certain materials, such as within a cellulose and/or synthetic fiber matrix. In some embodiments, at least one component of a chemiluminescent system is applied to one or more substrates as a dry formulation within a dry carrier material. Dry carrier materials may include any inert material to allow for flowable or dispersible formulations within application machinery, such as, but not limited to, sugars, minerals or salts thereof, starch, silica, clays, talc, micronized wood or other cellulose, gelatins, agars, and SAP. In one embodiment, an absorbent article includes a liquid permeable top sheet, a liquid-impermeable back sheet, a fibrous absorbent material that includes fibers treated with a luciferase, and a tissue sheet treated with a luciferin, in which the absorbent material and the tissue sheet are disposed between the top and back sheets in a configuration in which one of the chemiluminescent components is transferred to the other by an aqueous system moving from the top sheet toward the back sheet. For example, the treated tissue sheet may form a wrapping for an absorbent core that includes the treated absorbent material.

Photoluminescent Compound

The chemiluminescent systems may interact with a photoluminescent (e.g., fluorescent and/or phosphorescent) compound having a photoluminescent absorption wavelength range that overlaps with a chemiluminescent emission wavelength range of the chemiluminescent system, wherein the photoluminescent compound has a photoluminescent emission wavelength range that is different from the chemiluminescent emission wavelength range. As such, the photoluminescent compound can be used to “shift” the emission wavelength of the chemiluminescent system. For example, photoluminescence can be used to change or otherwise tailor the color (or other spectral quality) of the produced light.

While the chemiluminescent systems produce light, the chemiluminescence itself need not necessarily be in the visible spectrum. The chemiluminescence produces electromagnetic radiation in some wavelength range, but the disclosed embodiments are not limited to chemiluminescent emission in the visible range. Accordingly, in certain embodiments, a chemiluminescent system may produce chemiluminescent emission that is not in the visible wavelength range. In such embodiments, a photoluminescent compound may be used to shift the emission spectrum into the visible range.

The photoluminescent compound may be selected from the group consisting of a fluorescent compound and a phosphorescent compound. Fluorescent compounds can include, but are not confined to, xanthene derivatives such as fluorescein, rhodamine, Oregon green, eosin and Texas red; cyanine derivatives such as indocarbocyanine; naphthene derivatives; coumarin derivatives; oxadiazole derivatives such as pyridyloxazole; anthracene derivatives such as anthraquinone; pyrene derivatives such as Cascade blue; acridine derivatives such as proflavin, acridine orange, and acridine yellow; arylmethine derivatives such as auramine, crystal violet, and malachite green; tetrapyrrole derivatives such as porphin; bilirubin; phosphorescent compounds such as silver activated zinc sulfide and doped strontium aluminate; and so forth.

A photoluminescent compound can be disposed in any suitable material (e.g., an absorbent material) or component (e.g., a top sheet) for an absorbent article, or if incorporated into an absorbent article, in an adjacent layer (e.g., top sheet). Of importance is that the photoluminescent compound is in optical communication (at emission and excitation wavelengths) with the chemiluminescence. For example, a photoluminescent compound can be disposed on a back sheet of an absorbent article.

pH Buffer A pH buffer may be present in the materials and/or absorbent articles. The pH buffer can be configured to modify the spectral properties such as intensity of the chemiluminescent system. For example, pH control can be used to improve the efficiency of the chemiluminescence.

Exemplary improvements to the efficiency of the chemiluminescence include extending or otherwise modifying the duration of emission so as to provide a caregiver a desired amount of time to detect an insult. Accordingly, the pH buffer may be configured to increase the efficiency of the visible light from the chemiluminescent system upon contact with the aqueous system.

The pH buffer may be selected from the group consisting of sodium bicarbonate, sodium acetate, sodium citrate, sodium lactate, sodium lactate citrate, sodium borate, calcium acetate, calcium citrate, calcium bromide, calcium gluconate, calcium lactate, calcium lactate malate, calcium carbonate, calcium bicarbonate, and potassium dihydrogen phosphate. Calcium salts are particularly effective in increasing the efficiency of the chemiluminescence reaction.

The pH buffer can be disposed in any suitable material (e.g., an absorbent material) or component (e.g., a top sheet) for an absorbent article, or if incorporated into an absorbent article, in an adjacent layer (e.g., top sheet). Of importance is that the pH buffer contacts the chemiluminescent system upon insult. Therefore, the pH buffer should be disposed in an absorbent article such that it will be carried into contact with the chemiluminescent system upon insult.

### Treated Materials and Structural Elements

In one aspect of this disclosure, materials and structural elements treated with one or more reactive components of a chemiluminescent system are provided. In such embodiments, the treated material or structural element includes one or both reactive components of a bioluminescent system—that is, a luciferin and a luciferase.

The materials are those that are typically incorporated into an absorbent article. The materials may be absorbent or non-absorbent materials. Representative absorbent articles include child or baby diapers, adult diapers and incontinence products, feminine hygiene products, absorbent underpads, bandages and other wound care dressing articles, absorbent bed pads, and absorbent pet pads.

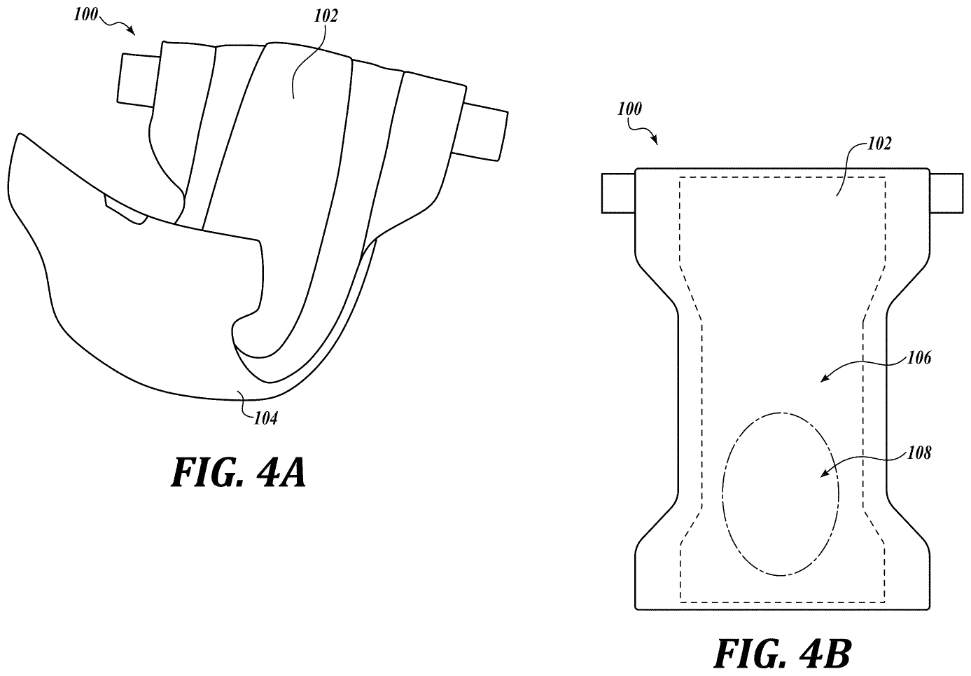

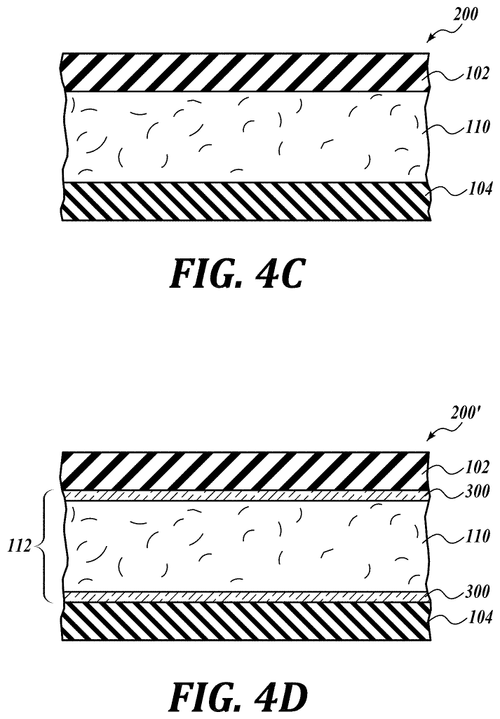

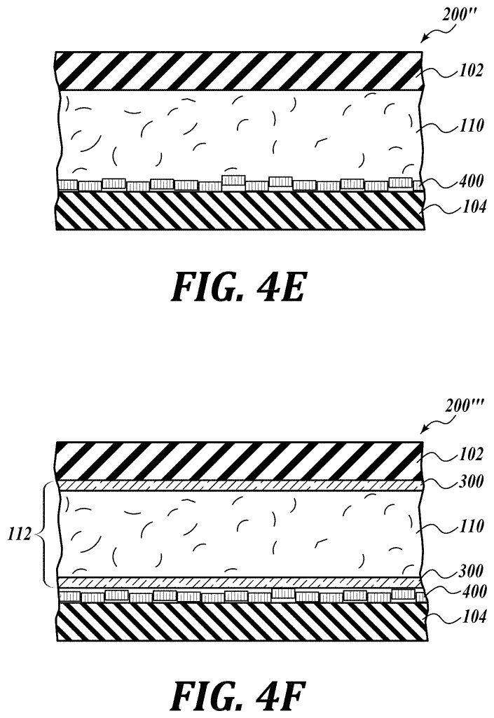

To illustrate example materials, a representative absorbent article is illustrated in FIG. 4A and FIG. 4B as a diaper 100, with FIG. 4B showing a simplified and somewhat schematic top view of the diaper in flattened form. However, the following description is applicable to all types of absorbent articles. The diaper includes a top sheet generally indicated at 102 and a back sheet at 104. Top sheet 102 is formed from a fluid-permeable material adapted to promote fluid transfer into the interior of the diaper, usually with minimal fluid retention by the top sheet. Example materials include nonwoven, fibrous sheets that incorporate synthetic and/or cellulosic fibers. In contrast, back sheet 104 is formed from a fluid-impermeable material to prevent any fluid leakage from the interior of the diaper. Also sometimes referred to as a “polysheet,” example back sheets include polyethylene sheets or films. Each of the top sheet and back sheet may be a composite and/or formed from one or more materials or layers, functioning together or independently to provide the fluid-permeable or fluid-impermeable characteristic of the sheet.

The interior of the diaper includes an absorbent region 106, and a target region 108 that generally indicates an area in which an insult is expected. The precise boundaries of regions 106 and 108 will vary with the design of the diaper or absorbent article.

FIG. 4C illustrates a cross-section 200 through the target region 108. In the cross-section 200, the diaper can be seen to include, in addition to top sheet 102 and back sheet 104, an absorbent material disposed between the top sheet and the back sheet, generally indicated at 110. The absorbent material may be any material, or combination of materials, suitable to absorb a fluid insult. For example, the absorbent material may include a fibrous matrix formed from cellulosic and/or synthetic fibers, SAP, and so forth.

A chemiluminescent system is also disposed in the diaper 100.

In some embodiments, the chemiluminescent system includes a luciferin component and a luciferase component, which are adapted to react with each other in the presence of an aqueous system to produce light. In some of such embodiments, the components are separately disposed within the diaper in a configuration in which one component is transferred to the other by an aqueous system, such as a fluid insult, for example moving from the top sheet toward the back sheet. The components may be disposed on, in, and/or throughout two or more materials incorporated into the diaper structure.

Example materials include both absorbent materials (e.g., materials that are typically incorporated into an absorbent core) and non-absorbent materials (e.g., materials that are typically incorporated elsewhere in the structure of the absorbent article), and combinations thereof. Examples of absorbent materials include fluff pulp, SAP, synthetic fibers, and so forth. Examples of non-absorbent materials include top sheet 102, back sheet 104, a tissue sheet or composition, one or more layers of material used in an acquisition and distribution layer (ADL), and so forth.

As noted above, Applicant's co-pending U.S. patent application Ser. No. 14/516,255 discloses fluff pulp compositions treated with a chemiluminescent system configured to produce visible light upon contact with an aqueous system, which may be incorporated into absorbent articles, for example as an absorbent material.

Treated Tissue Compositions

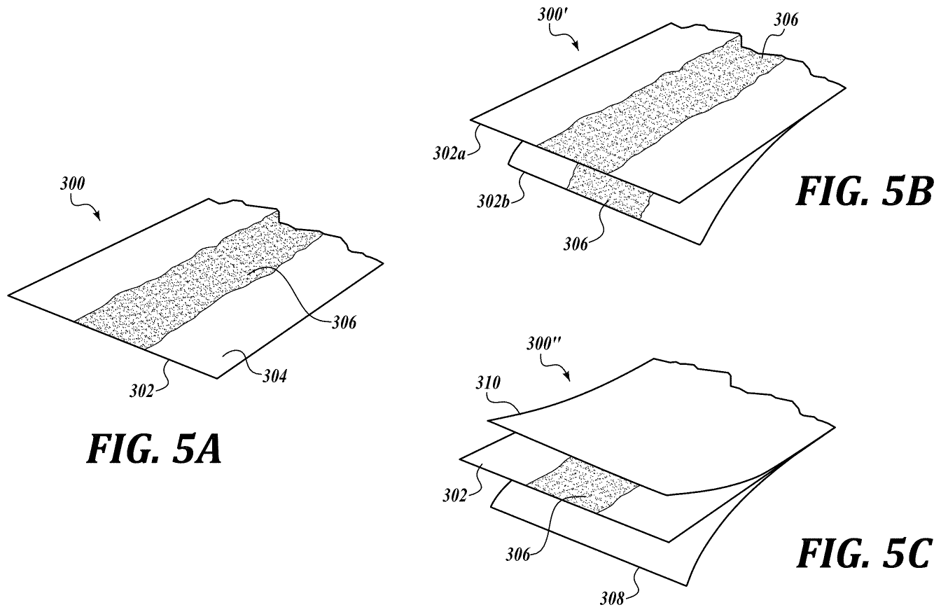

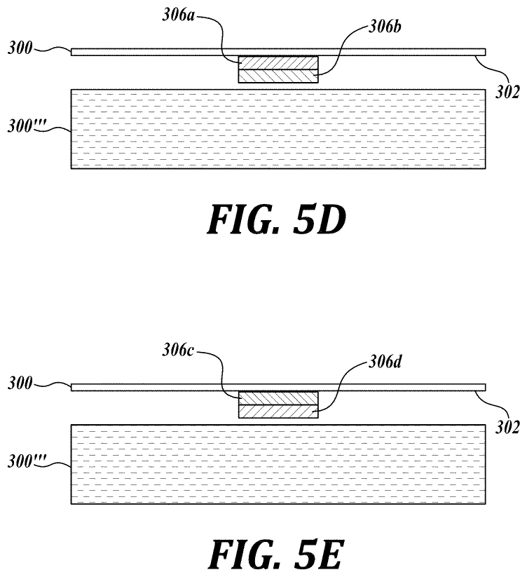

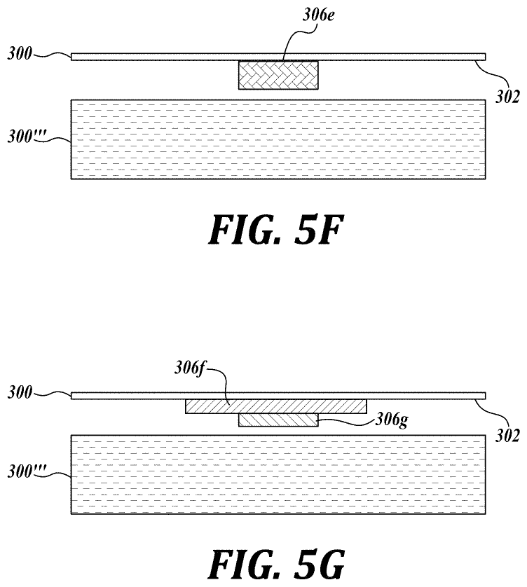

In one embodiment of a treated material in accordance with the present disclosure, a treated tissue composition is provided. FIG. 5A illustrates an example of such a tissue composition 300, which includes a liquid permeable tissue sheet 302. The tissue sheet includes fibers, such as fibers selected from cellulosic fibers, synthetic fibers, and combinations thereof, and may be of any construction and configuration suitable for incorporation into an absorbent article, for example as a wrapping material for an absorbent core.

Tissue sheet 302 has at least two opposed surfaces. Tissue sheet 302 has at least one surface 304 that is treated with at least one component of a chemiluminescent system, selected from a luciferin and a luciferase, with the component being retained on the treated surface. In some embodiments, the component is a luciferin selected from coelenterazine, a coelenterazine analog, dinoflagellate luciferin, bacterial luciferin, fungal luciferin, firefly luciferin, vargulin, furimazine, and combinations thereof. For example, in some of such embodiments, the component is coelenterazine. In some embodiments, the component is a luciferase selected from Gaussia luciferase, Renilla luciferase, Metridia luciferase, Oplophorus luciferase, dinoflagellate luciferase, copepod luciferase, and firefly luciferase. For example, in some of such embodiments, the component is Gaussia luciferase, Renilla luciferase, and/or Metridia luciferase. In some embodiments, the treated tissue composition incorporates both a luciferin and a luciferase, such that the light-producing reaction will be initiated upon contact of the treated tissue composition by a liquid insult. For example, in some of such embodiments, the luciferin is coelenterazine and the luciferase is Gaussia luciferase. In another example, in some of such embodiments, the luciferin is coelenterazine and the luciferase is Renilla luciferase. In yet another example, in some of such embodiments, the luciferin is coelenterazine and the luciferase is Metridia luciferase.

Although not necessary, a binder may optionally be used to assist retention of the chemiluminescent component on the tissue sheet, such as on the fibers of the tissue sheet. Generally speaking, a binder is any material or substance that holds or draws other materials together to form a cohesive whole mechanically, chemically, by adhesion or cohesion. It was found that while cellulose fibers are able to retain luciferin applied to the surfaces thereof mainly via molecular interaction, some synthetic fibers and/or surfaces are less able, or even unable, to do so. In such cases, it was found that one or more binders can facilitate the retention of luciferin on such materials, and also that binders provide can provide a similar benefit even on substrate materials able to retain luciferin. When the component is luciferin, suitable binders may include ethyl cellulose, methyl cellulose, nitrocellulose, polyurethane, and so forth, and various combinations thereof. Accordingly, in some embodiments, the component of the chemiluminescent system is retained on the surface of the tissue sheet by a suitable binder.

Some binders function by forming a coating or film over the surface of a substrate material, retaining the chemiluminescent component to the material, but also resisting penetration by an aqueous system to solubilize and/or release the chemiluminescent component for transfer to the site of chemiluminescence. This may manifest, for example, in a delay after an absorbent article is insulted before chemiluminescence is visible, lower chemiluminescent intensity upon an insult, such as a first insult, and so forth. Such a delay may be useful as a time-release mechanism in some embodiments. However, it may be preferable in many applications for chemiluminescence to be visible as soon as practicable following an insult. In some of such embodiments, the presence of a releasing agent can hasten or otherwise facilitate the release of a chemiluminescent component retained by a binder. Suitable releasing agents include poly(l-vinylpyrrolidone-co-vinyl acetate (PVPA), hydroxypropyl-β-cyclodextrin (HPBC), and so forth. Accordingly, some embodiments that include one or more binders also include one or more releasing agents.

Some embodiments include a luciferin, an aqueous or non-aqueous solvent to dissolve the luciferin, and an excipient (e.g., hydroxypropyl-β-cyclodextrin; ethanol; water soluble polymers selected from the group consisting of poly(ethylene glycol), poly(vinyl alcohol), partially hydrolyzed poly(vinyl alcohol), polyvinylpyrrolidone, poly(l-vinylpyrrolidone-co-2-dimethylaminoethylmethacrylate), poly(l-vinylpyrrolidone-co-vinyl acetate), and a combination thereof; sugars (mono-saccharides, poly-saccharides, and branched polysaccharides); cellulose and cellulose derivatives; minerals and salts thereof; etc.) adapted to facilitate the solubility of the luciferin in water. Some of such embodiments include a binder adapted to bind the luciferin to the substrate material. Some excipients, while inert in the chemiluminescent reaction, provide additional benefits. Such additional benefits include, for example, effects on solubility of a given component of the chemiluminescent system in various aqueous and non-aqueous solvent systems, effects on water availability in an absorbent article, retention effects (e.g., binders) on various substrates for one or both components of the chemiluminescent system, releasing effects (e.g., porous transfer agents) for one or both components of the chemiluminescent system, etc. Some such excipients may have multiple of these or other functions.

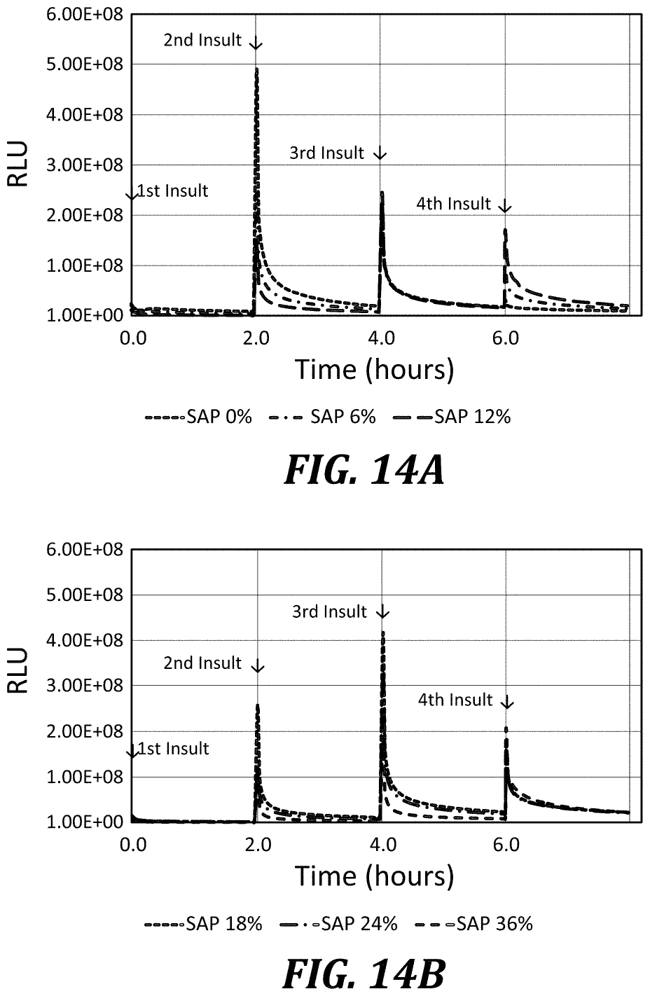

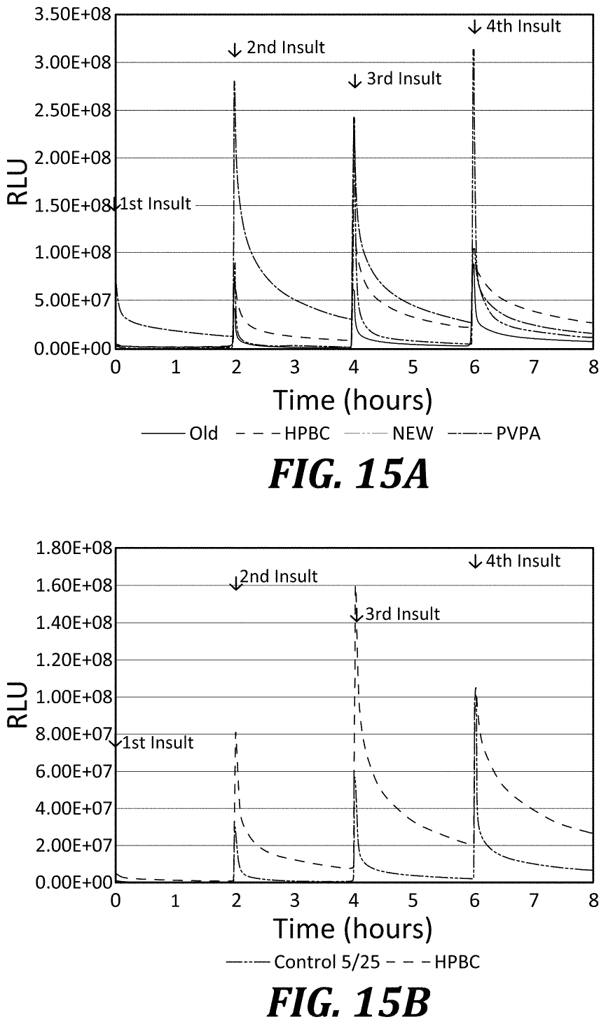

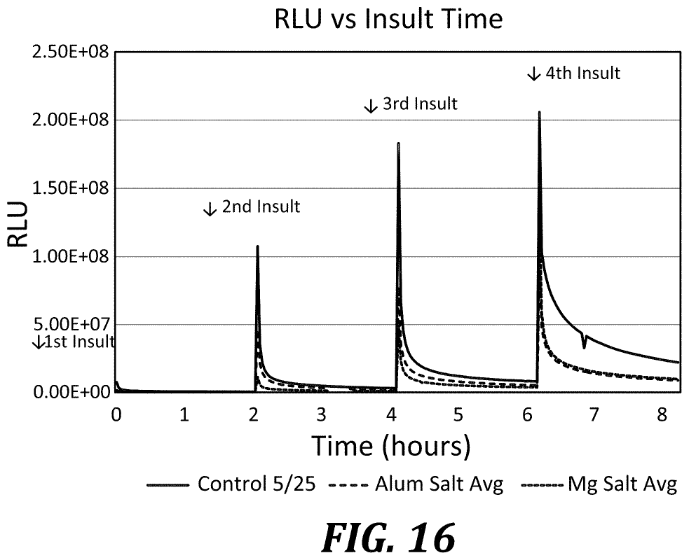

Provided here are some non-limiting examples within the scope of the present disclosure. In a first example, an excipient may be selected for its ability to affect the water availability within an absorbent article. Varying the relative amount of super absorbent materials (e.g., SAP) within a diaper structure will affect the amount of free water that is available to activate the chemiluminescent system by bringing the two components (i.e., luciferase and luciferin) together or otherwise allow the reaction to commence in an aqueous environment. SAP binds water, and less SAP allows for more free water and earlier peak photon production in an absorbent article with no SAP, staggered peak photon production with 12-24 weight % SAP, or later peak photon production with 36 weight % SAP (see FIGS. 14A & 14B; note that chemiluminescent system components and all other variables are constant). In a second example, an excipient may be selected for its ability to modulate the effects of a binder excipient when exposed to an aqueous system, thereby releasing one or more components of the chemiluminescent system for reaction within an absorbent article. Varying the amount or type of such a porous transfer agent excipient within a diaper structure will affect the availability of the bound component(s). In a diaper wherein the luciferin (coelenterazine) is disposed on an absorbent core tissue wrap, the inclusion of hydroxypropyl-β-cyclodextrin in the application formulation (see FIGS. 15A & 15B; “HPBC”) tempers the availability of the luciferin to react with the luciferase (Gaussia) disposed in the fluff pulp of the core, thereby delaying the peak production and allowing for longer sustained photon production (right shoulders on plot). Hydroxypropyl-β-cyclodextrin also aids in the solubility of coelenterazine in aqueous solutions for applying to the tissue core wrap structure. However, the inclusion of poly(l-vinylpyrrolidone-co-vinyl acetate) in the application formulation (see FIG. 15A; “PVPA”) provides robust availability of the luciferin, which is shown in bright early reactions following insults with an aqueous system that diminishes with each subsequent insult due to depletion of reactants. In a third example, an excipient may be selected for its ability to inhibit the reaction conditions of the chemiluminescent system. Varying the amount and type of salt treatment of the absorbent material can affect the pH of a resulting aqueous system following an insult to the absorbent article. By providing an unfavorable reaction environment up addition of multivalent minerals and/or salts thereof (see FIG. 16; aluminum and magnesium salts), the reaction is inhibited unless and until the pH conditions are changed over time (e.g., increased with the addition of a buffer component or urine insult(s)).

The area of surface 304 that is treated may have any desired size or shape. For example, in FIG. 5A, surface 304 is shown to have a treated area 306 in the form of a longitudinal strip having a width about ⅓ of that of the tissue sheet, such as may be applied by means of the methods disclosed herein, for example by streaming a formulation onto a continuous, moving sheet of tissue by means of a stationary nozzle in a tissue printing and/or production apparatus.

Several aspects of the treated tissue composition may be customized to its use, and specifically the manner (or manners) in which the treated tissue composition is incorporated into an absorbent article. As noted above, embodiments in which the treated tissue composition includes one reactive component would typically be incorporated into an absorbent article together with the other reactive component disposed elsewhere in the absorbent article—such as in another treated material or layer—in an arrangement in which one reactive component would be transferred to the other upon receipt of a fluid insult by the absorbent article, such as when the aqueous fluid of the insult moves from the top sheet toward the back sheet (for example), to thereby react and initiate the light-producing reaction.

As one example of such an absorbent article, the other reactive component may be disposed in the absorbent material of the absorbent article.

As noted above, in some diaper configurations, an absorbent core is a self-contained component placed into the diaper during production. Thus, one example use of the treated tissue composition, the absorbent materials of the absorbent core (e.g., fluff pulp, synthetic fibers, SAP, and so forth) may be enveloped or at least partially encompassed in the treated tissue composition. In such a configuration, a fluid insult would transfer a first reactive component from the absorbent core to a second reactive component, for example, located in the treated tissue composition (or from another location to the treated tissue composition or other placement of the second reactive component), to initiate the chemiluminescence.

Accordingly, FIG. 4D illustrates an example cross-section 200′ of the diaper 100 similar to that shown in FIG. 4C, but featuring an absorbent core 112 disposed between the top sheet 102 and back sheet 104. In the absorbent core 112, a treated tissue composition 300 is shown enveloping absorbent material 110.

The treated tissue composition may be incorporated into an absorbent article in a variety of manners other than as shown. For example, instead of (or in addition to) being used as a wrapping material for an absorbent core, a layer of the treated tissue composition may be placed in the diaper structure adjacent or at least partially encompassing the absorbent core, such as to reduce the tendency of loosened SAP particles to migrate from the absorbent core. In some embodiments of an absorbent core in accordance with this disclosure, an absorbent structure is at least partially encompassed by a treated tissue composition that incorporates a luciferin, wherein the absorbent structure incorporates a luciferase. In other configurations, the treated tissue composition (e.g., a layer, or layers, or discrete pieces) may be placed as desired in a diaper upstream of a location at which another component of the chemiluminescent system is disposed in a manner such that a fluid insult moving through the diaper encounters the treated tissue composition and transfers the reactive component of the tissue sheet to the other component, where the components react and chemiluminesce. In still other variants, the treated tissue composition may be placed in a diaper downstream of another component, so that the other component is transferred to the treated tissue composition by a fluid insult. In some variants, the treated tissue composition is placed adjacent or proximate to the back sheet, such as to optimize visibility of the chemiluminescence. Although the usual path for a fluid insult in an absorbent article is from the top sheet toward the back sheet, this is not always the case (for example, a fluid insult may travel laterally through the diaper structure). As such, the terms “upstream” and “downstream” are used herein for convenience, and do not require or imply a particular direction of flow relative to the absorbent article.

As such, the treated tissue sheet 302 may have any suitable dimensions. A standard baby diaper, which is discussed herein as a useful reference for size, is about 235 mm wide and about 350 mm long. However, it will be understood that although several of the dimensional aspects of the materials and structural elements disclosed herein are discussed with reference to a standard baby diaper, such a reference is provided only as an illustrative and non-exclusive example of an absorbent article, rather than as a limitation to such dimensional aspects. Moreover, although a standard baby diaper size is referenced, baby diaper sizes and dimensions range considerably, with such ranges differing to some extent among different manufacturers. Also, other absorbent articles within the scope of this disclosure may be larger than baby diapers (e.g., adult incontinence products, pet pads, bed pads), smaller than baby diapers (e.g., feminine hygiene products), have different dimensional aspects, and so forth. Accordingly, although some embodiments of the treated tissue sheet 302 (and other treated materials) are discussed as having certain dimensional ranges, such ranges are provided only as non-limiting examples.

As such, in some embodiments suitable for use with a standard-sized diaper, the width of the treated tissue sheet may range from 0.1 mm to about 235 mm. In an embodiment, the width of the treated tissue sheet ranges from about 0.1 mm to about 2 mm, from about 1 mm to about 10 mm, from about 1 mm to about 25 mm, from about 5 mm to about 50 mm, from 5 mm to about 100 mm, from about 25 mm to about 150 mm, from about 50 mm to about 200 mm, from about 50 mm to about 235 mm, or within any range within any of these ranges, and all other possible subranges. Of course, this may be wider or narrower depending on the diaper or configuration of the tissue sheet if used for wrapping. The length may also be as desired, for example, as needed to form a wrapping for the absorbent core, or up to the length of the absorbent article if a layer of the treated tissue composition is used.

Also, although typically thin and flexible, the treated tissue sheet 302 may be of any suitable thickness or basis weight. For example, in some embodiments, the basis weight of the tissue sheet may range from 10-1500 g/m² (also expressed as “gsm”). In an embodiment, the basis weight of the tissue sheet is in a range from about 10 g/m² to about 50 g/m², from about 25 g/m² to about 100 g/m², from about 50 g/m² to about 250 g/m², from about 100 g/m² to about 500 g/m², from about 250 g/m² to about 1000 g/m², from about 500 g/m² to about 1500 g/m², or within any range within any of these ranges, and all other possible subranges.

The treated tissue composition 300 may incorporate a broad concentration range of the component(s) of the chemiluminescent composition, with a suitable concentration determined by such factors as the surface area of the tissue sheet that is treated with the reactive component, the portion of the treated surface area expected to be exposed to a fluid insult, the desired intensity and/or duration of light, the nature of other reactive component(s) of the chemiluminescent system that are or will be incorporated in the diaper, and so forth. In accordance with the principles and concepts disclosed herein, an artisan will be able to determine an appropriate combination of factors for any configuration of an absorbent article with no more than reasonable experimentation. In some embodiments in which the reactive component is the luciferin coelenterazine, the treated tissue composition may comprise from 0.00002 to 20 weight percent coelenterazine based on a standard diaper size having a 235 mm width tissue sheet as a core wrapping wherein the tissue sheet has a mass of 1.32 gram. For example, in one such embodiment, the treated tissue composition comprises from 1 to 6 weight percent coelenterazine. In one embodiment, the treated tissue composition comprises from 0.00002 to 0.01 weight percent coelenterazine. In one embodiment, the treated tissue composition comprises from 10 to 20 weight percent coelenterazine. In one embodiment, the treated tissue composition comprises from 0.01 to 2 weight percent coelenterazine. In one embodiment, the treated tissue composition comprises from 2 to 10 weight percent coelenterazine.

The concentration of the component(s) in the treated tissue composition 300 is not particularly limited, except by the capacity of the tissue sheet. In general, it is usually economically preferable to use only as much of a component as is needed to generate a desired intensity or duration of chemiluminescence. However, the disclosure is not so limited, as it may be desirable to incorporate a greater amount of one or more components, such as to assure that sufficient quantity will react even after prolonged storage. Another way to express the concentration range of the reactive component(s) of the chemiluminescent system on the tissue sheet is by means of weight or mass of the component per surface area of the tissue sheet. Typically, the tissue sheet is produced as a continuous roll that is cut into standard lengths, such as a 12-inch length, with the width usually ranging from about 0.1 mm to the width of a standard diaper (about 235 mm), or more for larger diapers and incontinence products. In an embodiment in which the reactive component is the luciferin coelenterazine, the treated tissue composition may comprise 0.01 mg-25 g of coelenterazine per 12-inch length, with the weight of the coelenterazine per 12-inch length being less than or equal to the weight of a 12-inch length of untreated tissue sheet. In another embodiment in which the reactive component is the luciferin coelenterazine, the treated tissue composition may comprise up to 0.1-100 mg of coelenterazine per 12-inch length, with the weight of the coelenterazine per 12-inch length being less than or equal to the weight of a 12-inch length of untreated tissue sheet.

The tissue sheet 302 may be of any suitable configuration or composition. In one variant, a tissue sheet consists entirely of cellulosic fibers. In another variant, a tissue sheet comprises synthetic fibers.

Further, although the treated area 306 of surface 304 is shown as a longitudinal strip, the treated area (or areas) may have any desired form and dimension, such as a desired pattern, so that the chemiluminescence takes the form of such a pattern. For example, the treated area may be in the form of a strip that runs perpendicular or otherwise transverse to the longitudinal direction of the tissue sheet. A strip may be continuous or discontinuous (such as dotted or dashed), straight or curved or including curved and/or straight portions. The treated area may be in the form of one or more shapes or other forms, and so forth.

Also, the total treated surface area may be of any size relative to the surface area of the tissue sheet, such as ranging from about 0.003-100% of the area of the tissue sheet, with the lower limit representing a single dot of sufficient concentration to react to produce light, and the upper limit representing complete coverage of the tissue sheet surface. In this regard, the total treated area may be anywhere from 1 to 99% of the area of the surface, for example from 1-2%, 1-10%, 5-20%, 1-25%, 10-50%, 20-90%, or within any range within any of these ranges, and all other possible subranges.

The treated tissue sheet 302 is shown in FIG. 5A to have only one treated surface 304, but the disclosure is not so limited, as the opposed surface may also be treated, such as with the same or a different component. Moreover, the surface 304 may be treated with both components, such as in non-overlapping areas in some embodiments.

The treated tissue composition 300 is shown in FIG. 5A as a single ply of treated tissue sheet 302, but the disclosure is not so limited, as the treated tissue composition may incorporate multiple layers, for example a multilayered structure in which one layer is the treated tissue sheet. FIG. 5B illustrates an example embodiment of a treated tissue composition 300′ that is shown to include two tissue sheets 302a, 302b, that are treated with different components of the chemiluminescent system, such that the light-producing reaction will be initiated upon contact of the treated tissue composition by a liquid insult (a similar effect to embodiments in which a single tissue sheet is treated with more than one reactive component). In variations of this configuration, multiple plies of tissue sheet may each be treated with the same or different components of the chemiluminescent system. FIG. 5C illustrates another example embodiment of a treated tissue composition 300″ in which a treated tissue sheet 302 is sandwiched between two layers 308, 310 of liquid permeable material, such as untreated plies of a tissue sheet. Multilayered embodiments may be incorporated into an absorbent article in accordance with the principles discussed above.

In some embodiments, overlapping areas on surface 302 may be treated by applying one or both components in combined conditions that are substantially non-aqueous. Non-limiting examples of such embodiments include:
applying one of the components onto surface 302 in an aqueous-based solution to create a first treated area 306a, allowing that solution to substantially dry (i.e., insufficient water to allow for more than 5% of one or both components in the treated area to be consumed in a chemiluminescent reaction without further addition of water), and applying the other of the components onto surface 302 in any manner without water to create a second treated area 306b (see FIG. 5D);
applying one of the components onto surface 302 in a non-aqueous solvent-based solution to create a first treated area 306c, allowing that solution to substantially dry, and applying the other of the components onto surface 302 in a manner that limits water availability (i.e., by quickly drying or non-aqueous solvent systems) to create a second treated area 306d (see FIG. 5E); and
applying both of the components in a non-aqueous solvent-based solution onto surface 302 to create a single treated area 306e (see FIG. 5F).
   The treated areas 306a-d can be of any desired dimension, and the dimension does not need match (as shown in FIGS. 5D & 5E). Rather, in some embodiments, a first treated area 306f may have a larger dimension than a second treated area 306g (see FIG. 5G). One and/or the other component of the chemiluminescent system may comprise the application material forming first treated area 306f and second treated area 306g. The first treated area 306f and second treated area 306g may be completely overlapping (as shown in FIG. 5G for second treated area 306g), may be partially overlapping (not shown), or any other pattern. Furthermore, in some embodiments one and/or the other component of the chemiluminescent system may be comprised in a different absorbent article structural element 300′″, for example where structural element 300′″ is a fluff pulp core and structural element 300 is a tissue sheet. Depending on application method for treating the chemiluminescent component(s), any structural element within an absorbent article may be realized as structural element 300 and structural element 300′″. Note that FIGS. 5D-5G are shown in two-dimensional cross-sectional schematics.

Optionally, a treated tissue composition in accordance with this disclosure may include other additive materials described elsewhere herein, such as a photoluminescent compound, a pH buffer, a porous transfer agent, and so forth.

Indicator Particles

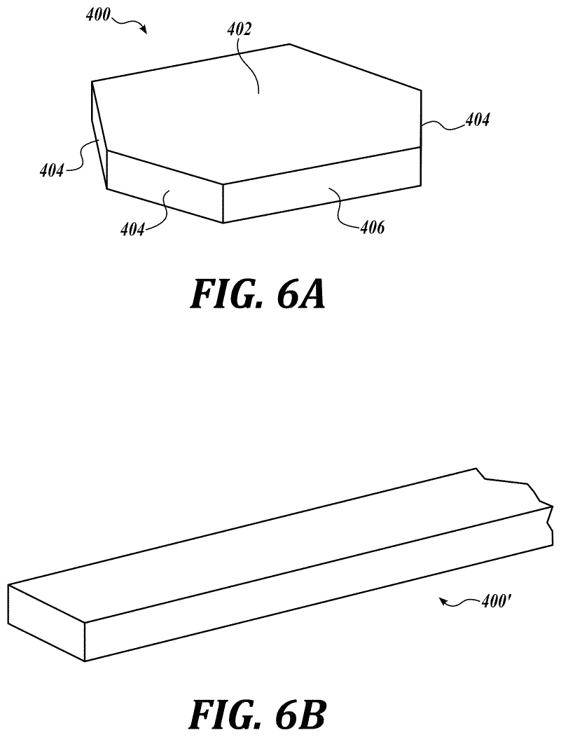

In another embodiment of a treated material in accordance with the present disclosure, an indicator particle is provided. FIG. 6A illustrates an example of such a particle 400, which is formed from hydrogen bonded cellulose pulp fibers and incorporates at least one component of a chemiluminescent system, selected from a luciferin and a luciferase, that is retained on the fibers. The component may be disposed on one or more surfaces 402 of the particle, and/or on the fibers throughout the particle. In some embodiments, the component is a luciferin selected from coelenterazine, a coelenterazine analog, dinoflagellate luciferin, bacterial luciferin, fungal luciferin, firefly luciferin, vargulin, furimazine, and combinations thereof. For example, in some of such embodiments, the component is coelenterazine. In some embodiments, the component is a luciferase selected from Gaussia luciferase, Renilla luciferase, Metridia luciferase, Oplophorus luciferase, dinoflagellate luciferase, copepod luciferase, and firefly luciferase. For example, in some of such embodiments, the component is Gaussia luciferase, Renilla luciferase, and/or Metridia luciferase. In some embodiments, the indicator particle incorporates both a luciferin and a luciferase, such that the light-producing reaction will be initiated upon contact of the indicator particle by a liquid insult. For example, in some of such embodiments, the luciferin is coelenterazine and the luciferase is Gaussia luciferase. As another example, in some of such embodiments, the luciferin is coelenterazine and the luciferase is Renilla luciferase. As yet another example, in some of such embodiments, the luciferin is coelenterazine and the luciferase is Metridia luciferase.

Although not necessary, a binder may optionally be used to assist retention of the chemiluminescent component on the indicator particle, such as on the fibers of the indicator particle. As noted above, when the component is luciferin, suitable binders may include ethyl cellulose, methyl cellulose, nitrocellulose, polyurethane, and so forth, and various combinations thereof. Accordingly, in some embodiments, the component of the chemiluminescent system is retained on the surface of the tissue sheet by a suitable binder.

Although not necessary, a porous transfer agent may be incorporated into the indicator particles. It was found that some porous media function to assist mass transfer of the reactive component(s) and/or the aqueous system in some applications, such as those in which the reactive components are disposed in separate locations, either on the indicator particles, on other treated materials, or otherwise in the absorbent article. For example, in the case of the luciferin coelenterazine, which can exhibit low water solubility, the greater the surface area treated with coelenterazine, the more available the reactant will be to luciferase. Porous media can provide greater surface area, as compared to non-porous and less porous materials. Moreover, hydrophilic porous materials can also facilitate water transfer to the reaction site for the light-producing reaction.

Unexpectedly, porous transfer agents were found to confer a benefit in some embodiments in which a binder is used. As noted above, some binders function by forming a coating or film over the surface of a substrate material, retaining the chemiluminescent component to the material, but also resisting penetration by an aqueous system to solubilize and/or release the chemiluminescent component for transfer to the site of chemiluminescence. This may manifest, for example, in a delay after an absorbent article is insulted before chemiluminescence is visible, lower chemiluminescent intensity upon an insult, such as s first insult, and so forth. However, it was found that in some of such embodiments, the presence of a porous transfer agent hastens or otherwise facilitates mass transfer, by ameliorating the aforementioned effect of some binders. In other words, functionally speaking, porous transfer agents, in some embodiments, were found to confer a similar benefit to releasing agents such as PVPA and HPBC.

Porous and hydrophilic media were found to be generally suitable for use as porous transfer agents. A non-exclusive list of porous transfer agents suitable for such use includes starch, amorphous silica, clay minerals, cellulose pulp fiber, cotton fiber, and synthetic polymer fiber. Owing to their ability to facilitate mass transfer, some embodiments may include a porous transfer agent even in the absence of a binder.

Within the aforementioned parameters, the indicator particle may have any desired size, shape, and/or form. The particle may be produced by cutting or otherwise partitioning a sheet of cellulose pulp, in which case the thickness and basis weight of the particle may be that of the sheet from which it is cut. For example, a Henion dicer may be used to dice a sheet of cellulose pulp into generally hexagonally-shaped particles such as that illustrated in FIG. 6A, which has two opposed surfaces 402, a thickness of about 1 mm, four sides 404 that are about 2 mm in length, and two sides 406 that are about 3 mm in length. There may be some variance in shape and/or dimensions owing to the partitioning method used.

FIG. 6B illustrates another example indicator particle 400′, in the form of an elongate strip that is 1 mm thick, about 2 mm wide, and about 350 mm in length. As explained below, the various forms of the indicator particles may be customized to their expected use and desired length (and other physical dimensions) in an absorbent article.

FIGS. 6A and 6B are illustrative in nature. As such, particles 400, 400′ are shown, for example, as having flat, planar sides and linear edges. Particles having these forms will likely include and exhibit various irregularities as a result of the material from which the particles are produced, the production method, and so forth. For example, particles 400 and 400′ may exhibit some curl and/or twist, and/or appear frayed along their edges, and so forth.

For convenience, the two main forms of indicator particles discussed herein may be distinguished by means of their aspect ratios, or the ratio of the particle's length to its width. Particles with aspect ratios less than about 1.5, such as indicator particle 400 in FIG. 6A, are referred to herein as “flakes,” whereas particles with aspect ratios greater than or equal to about 1.5, such as the indicator particle 400′ in FIG. 6B, are referred to herein as “strips.” This distinction may be useful in some applications, for example if a particular shape of a particle is preferable to another, although the principles of use are generally otherwise the same. In some embodiments, a flake will have a width greater than its thickness, and a length greater than its width, as in particle 400. The particle 400 has two opposed faces, the area (e.g., the product of the length and width) of each of which is about 14 mm² in the illustrated example, but can range considerably. For example, in some embodiments, the area of such a particle ranges between 0.1 mm²-300 mm². In some embodiments, the area of such a particle ranges between 1 mm²-10 mm². In some embodiments, the area of such a particle ranges between 8 mm²-30 mm². In some embodiments, the area of such a particle ranges between 30 mm²-150 mm². In some embodiments, the area of such a particle ranges between 50 mm²-150 mm². In some embodiments, the area of such a particle ranges between 100 mm²-300 mm². Similarly, the shape of a flake may vary from that shown in FIG. 6A. For example, the shape may be square, circular, regular or irregular polygonal, and so forth. The dimensions and shape of a strip can similarly vary. For example, the width of particle 400′ (a strip) is 2 mm in the illustrated example, but varies in some embodiments from about 0.5 mm-2.5 mm. The thickness is 1 mm, but varies in some embodiments from about 0.05 mm-2.0 mm. The cross-section is about 2 mm², but varies in some embodiments from about 0.01 mm²-200 mm². The length can also vary from about 1 mm to about 800 mm. While 350 mm is approximately the length of a standard diaper, longer lengths (such as for larger diapers and adult incontinence products, for example) may be used, up to and even beyond 800 mm. Also, the cross-sectional dimensions are shown to be constant along the length of strip 400′, but strips may include non-constant cross-sections, and twist or curl along their lengths.

Any suitable partitioning method may be used, depending on the size and shape desired. Any suitable material that incorporates hydrogen-bonded cellulose pulp fibers may be used. For example, pulp sheets having a basis weight ranging from 10-850 gsm may be used. In some embodiments, the material also incorporates synthetic fibers, having fiber diameters ranging from 1 micron-100 microns.

Somewhat similar to the treated tissue compositions in accordance with the present disclosure, several aspects of the indicator particles may be customized to their use, and specifically the manner(s) in which they are incorporated into an absorbent article. One example use of the particle 400 (flakes) is illustrated in FIG. 4E, which illustrates an example cross-section 200″ of diaper 100 similar to that shown in FIGS. 4C and 4D, but featuring a plurality of flakes 400 arranged in a single layer between the absorbent material 110 and the back sheet 104, wherein the absorbent material 110 is shown disposed between the back sheet 104 and the top sheet 102. A similar example embodiment (not illustrated) employs one or more of particle 400′ (strips), placed parallel to the length of the diaper in a single layer between the absorbent material 110 and the back sheet 104. In yet another example embodiment, a plurality of flakes 400 are uniformly distributed throughout the absorbent material 110.

Like the treated tissue compositions, the indicator particle (or particles) may be incorporated into an absorbent article in a variety of manners other than as shown. For example, the indicator particles may be disposed elsewhere in the diaper, such as in another layer or location between the top sheet and the back sheet, for example depending on whether the particles incorporate a luciferin, a luciferase, or both; the desired location within the diaper for the chemiluminescent reaction to take place upon insult (e.g., whether the chemiluminescent component(s) incorporated in the particles will be transferred to a different location by a fluid insult, or whether another chemiluminescent component will be transferred to the particles); the desired intensity of the chemiluminescence; the desired shape of the chemiluminescent area (e.g., a strip, or plurality of strips, may exhibit chemiluminescence as corresponding glowing stripes); and so forth. The flake form of the particle may be useful not only as a substrate material for a chemiluminescent component, but also in terms of absorbent function. As disclosed in U.S. Pat. No. 9,617,687, for example, prismatoid pulp particles may be used as, or in, an ADL for a diaper. The '687 patent explains that when such particles are distributed as a layer within an ADL, the particles can maintain a space between the top sheet of the ADL and the storage layer thereof, with the gaps and void spaces between and among adjacent particles forming channels through which fluid can flow to the storage layer. In a somewhat similar manner, flakes 400 may be incorporated into an ADL for an absorbent article, while also providing one or more components of the chemiluminescent system.

The indicator particles 400 may incorporate a broad concentration range of the component(s) of the chemiluminescent composition, with a suitable concentration determined by factors discussed above with regard to treated tissue composition 300.

In one embodiment of the particles 400 (flakes), the reactive component is the luciferin coelenterazine, and the particles include the component in a concentration of less than 0.50 weight percent. In one embodiment of the particles 400, the reactive component is Gaussia luciferase, Renilla luciferase, and/or Metridia luciferase, and the particles include the component in a concentration less than 0.01 total weight percent. In one embodiment of the particles 400′ (strips), the reactive component is the luciferin coelenterazine, and the particles include the component in a concentration less than 0.50 weight percent. In one embodiment of the particles 400′, the reactive component is Gaussia luciferase, Renilla luciferase, and/or Metridia luciferase, and the particles include the component in a concentration less than 0.01 total weight percent. However, in either (or any other) form, the indicator particles may comprise from 0.00002-20.0 weight percent coelenterazine and/or from 0.003-10.0 weight percent Gaussia luciferase, Renilla luciferase, and/or Metridia luciferase, and/or a suitable amount of one or more luciferins and/or luciferases. Some sample weight percent ranges for coelenterazine in such a particle include 0.00002-0.01, 0.01-0.20, 0.20-5.0, and 5.0-20, or within any range within any of these ranges, and all other possible subranges. Some sample weight percent for the total amount of Gaussia luciferase, Renilla luciferase, and/or Metridia luciferase in such a particle include 0.0003-0.001, 0.001-0.1, 0.1-2.0, and 2.0-10, or within any range within any of these ranges, and all other possible subranges.

The concentration or amount of the component(s) in the indicator particles is not particularly limited, except by the capacity of the particles themselves. In general, it is usually economically preferable to use only as much of a component as is needed to generate a desired intensity or duration of chemiluminescence. However, the disclosure is not so limited, as it may be desirable to incorporate a greater amount of one or more components, such as to assure that sufficient quantity will react even after prolonged storage. Another way to express the concentration or quantity range of the reactive component(s) of the chemiluminescent system on the indicator particles is by means of total quantity of the component used in an absorbent article. In one embodiment of an absorbent article (in the form of a diaper) that includes a plurality of about 65 particles of similar size and dimension to particles 400, or about 0.7 g total weight of particles, collectively containing about 1.0 mg of coelenterazine, and about 0.25 mg Gaussia luciferase, Renilla luciferase, and/or Metridia luciferase. In other variants, the plurality of indicator particles used in an absorbent article may collectively contain coelenterazine in a total quantity ranging from 0.0001-20.0 mg and/or Gaussia luciferase, Renilla luciferase, and/or Metridia luciferase in a total quantity ranging from 0.00003-20.0 mg. In other variants, the plurality of indicator particles used in an absorbent article may collectively contain coelenterazine in a total quantity ranging from 0.0001-20.0 mg, 0.001-20.0 mg, 0.01-20.0 mg, 0.1-20.0 mg, or 1-20.0 mg and/or Gaussia luciferase, Renilla luciferase, and/or Metridia luciferase in a total quantity ranging from 0.00003-20.0 mg, 0.0003-20.0 mg, 0.003-20.0 mg, 0.03-20.0 mg, or 0.3-20.0 mg.

Although the absorbent articles discussed above incorporate a plurality of similarly configured indicator particles, the disclosure is not so limited, as variations of an absorbent article in accordance with the present disclosure may include a plurality of particles that are not all similarly configured, such as an absorbent article that includes particles of two or more different forms, sizes, or shapes, and/or particles that separately incorporate different components of the chemiluminescent system, such as a first plurality of particles that incorporate a luciferin and a second plurality that incorporate a luciferase, and so forth.

It will be evident that multiple treated materials, and/or multiple types of treated materials, such as the aforementioned treated tissue composition and indicator particles, may be incorporated into an absorbent article. For example, one component of a chemiluminescent system may be provided by the treated tissue composition, and another component may be provided by the indicator particles, and disposed in the diaper such that one component is transferred to the other upon insult of the diaper with an aqueous system, to initiate the chemiluminescent-producing reaction between the components.

An illustrative example of such a configuration is provided in FIG. 4F, which shows a cross-section 200′″ of diaper 100 similar to those shown in FIGS. 4C-4E, but featuring an absorbent core 112 disposed between the top sheet 102 and back sheet 104, with the absorbent core formed from absorbent material 110 enveloped by treated tissue composition 300. This example also shows a layer of particles 400 (flakes) arranged in a single layer between the absorbent core 112 and the back sheet 104. In use, a fluid insult moving generally from the top sheet 102 toward the back sheet 104 will encounter the treated tissue composition 300, and transfer the chemiluminescent component disposed on the treated tissue composition as it continues moves toward the back sheet, to initiate the chemiluminescent reaction when it reaches the particles 400. In variations of such a configuration, one or more of the structural elements (that is, the tissue sheet and the indicator particles) may include more than one of the reactive components, such as to better assure that the transfer of one reactive component to the other will occur under a variety of conditions, to initiate chemiluminescence.

Optionally, an indicator particle in accordance with this disclosure may include other additive materials described elsewhere herein, such as a releasing agent, a photoluminescent compound, a pH buffer, and so forth.

Treated Absorbent Cores

As noted above, advances in SAP technology have allowed the design of absorbent core configurations in which fluff pulp contributes less to the absorbent capability of the core and more to providing a fibrous structure for fluid distribution and/or to stably retain SAP. These functions may, in some cases, be provided by synthetic fibers, leading to the development of absorbent cores that partially or even completely substitute synthetic fibers for the natural (e.g., cellulose) fibers historically used. Accordingly, in another embodiment of a treated material in accordance with the present disclosure, an article is provided that includes synthetic fibers and at least one of a luciferin and a luciferase. In some embodiments, the synthetic fibers form a nonwoven, absorbent matrix, and thus the article may be suitable for use in, or as, an absorbent core (such as a fluff-less absorbent core) for an absorbent article.

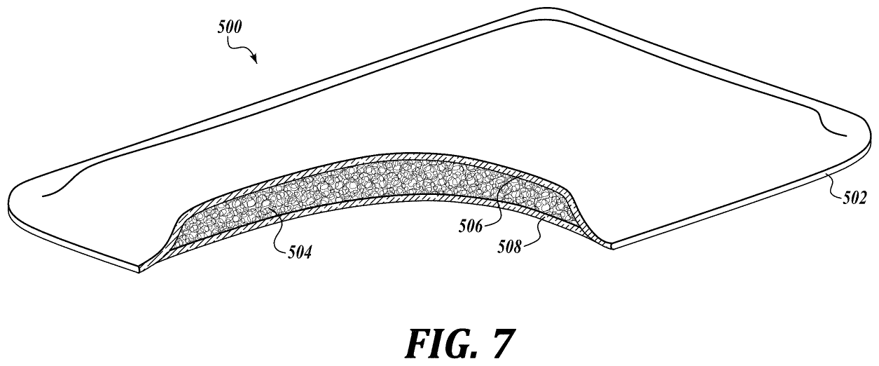

FIG. 7 illustrates an example embodiment of such an article 500, in the form of an absorbent core 502. Shown in partial cut-away, absorbent core 502 includes synthetic fibers 504 arranged to form an absorbent matrix and sandwiched between two sheets 506, 508 of material, which thereby form a self-contained component suitable for incorporation into an absorbent article. The two sheets may be of the same material or different materials, but typically will be fluid-permeable materials such as a nonwoven web or sheet, such as a tissue sheet. In some embodiments, one of sheets 506, 508 will be a liquid permeable top sheet, and the other will be a liquid-impermeable back sheet, and so forth.

A broad variety of synthetic fibers may be used. For example, the synthetic fibers may include fibers composed of one or more of polypropylene or other thermoplastic polymers, bicomponent fibers, elastomeric polymer fibers, and mixtures thereof. Several example synthetic fiber materials suitable for use are disclosed in U.S. Pub. No. 2007/0142803 of Soerens et al., the complete disclosure of which is incorporated herein by reference. Some embodiments additionally include natural fibers (e.g., cellulose fibers) together with the synthetic fibers, for example in a blend of the two types of fibers in the same fibrous matrix, or separately disposed in discrete layers or portions, and so forth. In some embodiments, such as in a fluff-less core, the fibers are entirely synthetic fibers.

Article 500 incorporates at least one component of a chemiluminescent system, selected from a luciferin and a luciferase. In the form of absorbent core 502, the chemiluminescent component may be disposed on and/or among the synthetic fibers, on a surface of or integrated into one or both sheets 506, 508, disposed on SAP particles that may be distributed throughout the synthetic fibers, and so forth. Absorbent core 502 may include additional structural elements, such as a distribution layer disposed between one of sheets 506, 508 and the synthetic fibers 504, with the chemiluminescent component disposed on or within such a layer, and so forth. In some embodiments of article 500, the chemiluminescent component is a luciferin selected from coelenterazine, a coelenterazine analog, dinoflagellate luciferin, bacterial luciferin, fungal luciferin, firefly luciferin, vargulin, furimazine, and combinations thereof. For example, in some of such embodiments, the component is coelenterazine. In some embodiments, the chemiluminescent component is a luciferase selected from Gaussia luciferase, Renilla luciferase, Metridia luciferase, Oplophorus luciferase, dinoflagellate luciferase, copepod luciferase, and firefly luciferase. For example, in some of such embodiments, the component is Gaussia luciferase. For example, in some of such embodiments, the component is Renilla luciferase. For example, in some of such embodiments, the component is Metridia luciferase. In some embodiments, the article incorporates both a luciferin and a luciferase, such that the light-producing reaction will be initiated upon contact of the article by a liquid insult. For example, in some of such embodiments, the luciferin is coelenterazine and the luciferase is Gaussia luciferase, Renilla luciferase, or Metridia luciferase and combinations thereof. In an embodiment that takes the form of an absorbent core as shown in FIG. 7, the luciferin and luciferase are disposed in different locations or materials of the absorbent core, such as with the luciferin disposed on the surfaces of the synthetic fibers, and the luciferase distributed throughout the synthetic fibers. In another such embodiment, the luciferase is distributed throughout the synthetic fibers and the luciferin is disposed on or integrated within one or both of sheets 506, 508. The concentrations and/or quantities of the chemiluminescent component(s) incorporated into the article 500 are not particularly limited, and may be consistent, such as on a per-diaper basis, with those described with respect to the other treated materials herein.

Optionally, an article (such as an absorbent core) in accordance with this disclosure may include other additive materials described elsewhere herein, such as a photoluminescent compound, a pH buffer, a binder, a porous transfer agent, a releasing agent, and so forth.

Encapsulated Chemistry

In a variant of some embodiments of the treated materials discussed above, a composition includes at least one component of a chemiluminescent system as encapsulated particles. In other words, in such embodiments, particles that include one component of a chemiluminescent system (such as particles of one or materials that have been treated with the chemiluminescent component, and/or the chemiluminescent component itself in particle form) each have a coating that covers the entire surface of the particle, with the coating being water soluble and/or water permeable. In a non-limiting, illustrative example, luciferase, in particle form (such as in powder or other ground form) is encapsulated, for example as water soluble capsules and/or micro-capsules, in coating materials such as sugars and polysaccharides (e.g., starch, dextrin, etc.), gums, water soluble polymers (e.g., PVOH), gelatin and other amino acid or protein-based materials, super absorbent materials, porous media, and so forth. In another non-limiting, illustrative example, luciferin-treated particles such as the aforementioned dice, or even smaller particles of a treated substrate material, are encapsulated, such as using one or more of the aforementioned coating materials. In compositions in accordance with the present disclosure, encapsulated particles collectively include a predetermined quantity of a first chemiluminescent component, and further include a predetermined quantity of a second chemiluminescent component, such that the predetermined quantities of the respective components are those sufficient to produce light of a desired intensity in the presence of an aqueous system. In some of such compositions, both of the first and second chemiluminescent components are encapsulated, such as separately encapsulated. Encapsulating one or both chemiluminescent components may provide stability and/or longevity of the chemistry in some applications, and/or ease of handling when incorporating the component(s) into an absorbent article, and so forth. Such compositions may, in some embodiments, allow an end user to incorporate a chemiluminescent system into a standard absorbent article, and so forth.

Such compositions of partially or wholly-encapsulated chemiluminescent systems may be incorporated into absorbent articles in a variety of manners, such as dispersing the encapsulated component into an absorbent material such as a fibrous matrix, for example in a similar manner in which particulate SAP is dispersed in a fibrous matrix, with the other chemiluminescent component disposed elsewhere in the absorbent article, such as in or on a treated material or structural element, in accordance with other embodiments discussed herein. In two-component chemiluminescent systems in which both components are encapsulated, both may be dispersed throughout the same fibrous matrix, and so forth.

Other Treated Materials

Consistent with the concepts and principles discussed above and elsewhere herein, other treated materials and structural elements are within the scope of this disclosure. For example, in some embodiments, a poly back sheet is treated with one or more chemiluminescent components, in a manner conceptually similar to the treated tissue compositions discussed above. In some embodiments, a fluid-permeable material may be treated with one or more chemiluminescent components, to produce a carrier substrate for the component(s) for inclusion into an absorbent article during production, such as in a manner that integrates the inclusion of the treated carrier substrate into a standard production process for an absorbent article.

The manner in which the chemiluminescent system is disposed in or on such treated materials and/or structural elements may consider factors such as the nature of the intended use of an absorbent article incorporating the treated material or element, the application technology employed, user safety, principles of efficient and/or economical manufacturing, and so forth.

To illustrate, FIGS. 9A-9J show different example embodiments of a representative structural element suitable for incorporation into an absorbent article (full outline not shown). Each depicts a simplified and somewhat schematic top view of an embodiment of structural element 900. The structural element 900 itself is shown as a rectangle and described in the form of an absorbent core of suitable size and dimension, but the following description is applicable to materials and/or other structural elements that may be incorporated into an absorbent article, such as a top sheet, a back sheet, a tissue sheet, a treated layer of liquid permeable material, and so forth, which may have other shapes/dimensions or be similar to that shown.

Structural element 900 includes a first surface 902, which in turn includes a treated area 904, with the treated area having been treated with at least one component of a chemiluminescent system adapted to react in the presence of an aqueous system to produce light. Although the treated area may not necessarily be visually discernable from the remaining untreated portion(s) of the first surface, for the sake of explanation it is shown as such. The treated area may be produced in any manner disclosed herein, such as via application of the at least one component to the surface by printing, streaming, and so forth.

The treated area may have any desired configuration. For example, the treated area may cover all, or a part, or parts, of the surface. In many applications, however, a substantial portion of the surface may not be utilized, in that certain areas of the surface may not correspond to a target region in which a fluid insult is expected. Also, as many absorbent articles transfer fluid received from an insult, it may not be necessary to treat the entirety of an area that corresponds to a target region, as it may be expected that the fluid front from an insult may wick toward and then encounter a treated area, in order to trigger the light-producing reaction.

Thus, the treated area may be sized and shaped to provide the desired effect during use, while reducing the amount of chemiluminescent material(s) used. The example embodiment shown in FIG. 9A shows a treated area 904 as a continuous shape, specifically in the form of a strip 906 running the length of the element 900. As the width of the strip is substantially less than the width of the surface 902, such a configuration may represent corresponding cost savings relative to a configuration in which the entire surface 902 is treated. In example variations of this configuration, a first chemiluminescent material (i.e., component, such as either a luciferin and/or a luciferase) is applied to surface 902 in strip 906, and a second chemiluminescent material (i.e., component, such as the other or both of a luciferin or a luciferase) is applied to the entire surface 902, to another area of surface 902 (not shown), to the material making up structural element 900, or another structural element (not shown). FIG. 9B shows another example embodiment in which treated area 904 is shown as a pattern of discrete portions, specifically a discontinuous strip 908 running a portion of the length of the element 900. A discontinuous strip having the same width as a continuous strip may require comparatively less chemiluminescent material, and even less if the overall length of the strip is shorter. The disposition of a first chemiluminescent material (i.e., component, such as either a luciferin and/or a luciferase) in such an example can be applied to surface 902 in discontinuous strip 908, and a second chemiluminescent material (i.e., component, such as the other or both of a luciferin or a luciferase) is applied to the entire surface 902, to another area of surface 902 (not shown), to the material making up structural element 900, another structural element (not shown). In some embodiments like that shown in FIG. 9B, the luciferin and luciferase components may be applied together or separately in the same treated area 904 as the identical discrete portions of discontinuous strip 908 or individually such that neighboring discrete portions harbor different components, with or without all of the material of surface 902 or another structure being treated with at least one chemiluminescent material (i.e., component, such as either a luciferin and/or a luciferase).

In FIGS. 9A and 9B, the treated area is much smaller than the area of the surface. Moreover, in FIG. 9B, the treated area is proportionately smaller than the treated area of FIG. 9A. In these and the following embodiments, the treated area may be configured as desired to cover a total area that is less than the area of the first surface. For example, the treated area may be anywhere from 1 to 99% of the area of the surface, for example from 1-2%, 1-10%, 5-20%, 1-25%, 10-50%, 20-90%, or within any range within any of these ranges, and all other possible subranges.

A treated area consisting of a pattern of discrete portions may allow coverage of a large portion of the surface 902 while using less chemiluminescent material than complete, continuous treatment of the same sized area. An example of such a configuration is shown in the example embodiment illustrated in FIG. 9C, in which treated area 904 is in the form of an arrangement of shapes—specifically, an arrangement of star shapes 910—that extend over most of the area of the surface 902 (as shown) or only a targeted area of a structural element 900 (not shown). Other shapes or patterns, including mixtures of different shapes and/or irregular patterns, could be utilized in treated area 904. The number of individual shapes can be as desired, for example to correspond to an amount of fluid suitable to trigger light production. The individual chemiluminescent components can be disposed in shapes 910 as described above for the discrete portions of FIG. 9B or as otherwise described above.

The configuration of the treated area may consider the nature and expected use of the absorbent article in which the structural element 900 is used. In the case of a diaper or adult incontinence pad, for example, there is typically a localized region in which a fluid insult can be expected during use, as these articles are generally restricted from movement relative to the wearer. Diaper 100, shown in FIG. 4B, schematically illustrates this with target region 108. As such, the treated area of a structural element 900 suitable for diaper 100 (such as shown in FIG. 9A or 9B, for example) may correspond to the size and/or shape of target region 108, or a portion thereof, or a portion of one or more structural elements that overlap or are disposed near target region 108.

In some embodiments, a diaper or similar absorbent article may be configured to handle multiple insults (e.g., two or three sequential insults, or more than three) before changing is needed, such as by incorporating an absorbent core of appropriate construction. In general, fluid from an insult spreads laterally outward through the absorbent core from the initial point of insult, owing to the wicking capabilities of the materials used in the absorbent core, before it is stored. The fluid front from subsequent insults then wicks further away from the initial point of insult until it reaches unused SAP in the absorbent core. As such, the target region in such multiple-use configurations can be thought of as including a plurality of concentric, generally ring-shaped regions.

The arrangement of the treated area may accordingly be configured to indicate not only that an absorbent article has been used, but also the extent to which the absorbent article has been used, or lack thereof.

An example of such a configuration is shown in FIG. 9D, in which surface 902 of an embodiment of structural element 900 includes a target region 912, which corresponds to a region in which one or more fluid insults are expected during use of an absorbent article into which the structural element is incorporated. For the sake of brevity, target region 912 is shown to consist of three concentric regions (914, 916, 918) corresponding to the extent to which the fluid front from three sequential insults is expected to wick, respectively. Treated area 904, in the form of a discontinuous strip, is shown to partially overlap the target region, and moreover includes discrete portions or segments 920 that correspond to each of the three concentric regions.

Although the boundaries of the concentric regions will vary with the design of the absorbent article, the intended user, and so forth, the treated area is configured such that the fluid front from sequential insults will first encounter the inner pair of segments 920 of the treated area, before encountering the outer pairs of segments. The pattern of chemiluminescence produced thereby may thus visually indicate whether the diaper has received one, two, three, or more insults.

In configurations similar to that as shown in FIG. 9D, in which each of the portions of the treated area only overlap a corresponding portion of the target region, the different portions of treated area 904 may be configured to produce visual light that differs in at least one visual respect from each other. For example, the outer segments may be configured to produce light of a different (e.g., greater) intensity as compared to the inner segments, and/or light of a different color, duration, and so forth, such as by means of varying the chemistry applied to the different portions of treated area 904 in accordance with methods disclosed herein (e.g., via the use of a photoluminescent compound, differing concentrations of at least one chemiluminescent component, treatment coat weight, or buffers affecting efficiency of the chemiluminescent reaction).

For example, by varying the concentration of at least one chemiluminescent component, such as a luciferase or a luciferin, in treated area 904 or from treated area 904 to treated area 904 progressing through the concentric regions, the speed and/or intensity of the chemiluminescent reaction can be manipulated. This arrangement allows for precision in the amount and/or duration of the light production visible to a caregiver for example, such as early/fast glowing (articles needing immediate replacement), slow/later glowing (articles designed for longer wearing periods), shorter duration (articles that are more likely to have a detection by a caregiver), and longer duration (articles that are more likely to have a detection by a caregiver or to signal full capacity has been reached). The variance of the at least one chemiluminescent component from treated area to treated area can be from 1:1 to 1:100 and all ranges there between, including, but not limited to, 1:1.1, 1:1.2, 1:1.3, 1:1.4, 1:1.5, 1:1.6, 1:1.7, 1:1.8, 1:1.9, 1:2, 1:3, 1:4, 1:5, 1:6, 1:7, 1:8, 1:9, 1:10, 1:20, 1:30, 1:40, 1:50, 1:60, 1:70, 1:80, 1:90, and 1:99, or within any range within any of these ranges, and all other possible subranges. Such variations may enhance the nature of the luminescent indicator, which may allow the caregiver to more easily determine the status of the absorbent article. Alternatively, in some embodiments, the segments 920 may be disposed in only one or two of the concentric regions (e.g., only in region 918 (see FIG. 9H with segments 934), or only in region 916 (see FIG. 9I with segments 936), or in both regions 916 and 918), depending on the desired pattern of insult/wetness indication. Likewise, the individual chemiluminescent components can be disposed in segments 920, 934, and 936 as described above for the discrete portions of FIG. 9B, shapes of FIG. 9C, or as otherwise described above.

Another configuration suitable for a multiple-use absorbent article is shown in FIG. 9E, in which surface 902 of an embodiment of structural element 900 also includes a target region 912 having multiple concentric regions, but including a treated area 904 of two discrete portions 922 that are each shaped to have a surface area that increases with the expected wicking distance of the fluid front from multiple insults, which may thus produce a larger and larger area of luminescence as the absorbent article receives sequential insults. Unlike the configuration shown in FIGS. 9D, 9H, and 9I, portions 922 are shown to overlap all of the multiple concentric regions, and thus may be suitable in an embodiment in which the boundaries of the regions may not be as definite and/or predictable. The individual chemiluminescent components can be disposed in discrete portions 922 as described above for the discrete portions of FIG. 9B, shapes of FIG. 9C, or as otherwise described above.

In some applications, it may be preferable to locate the treated area (or portions thereof) away from a target region in which one or more fluid insults are expected. For example, absorbent articles such as bed pads are designed to cover a large area rather than being a worn item. Locating the treated area away from a target region may reduce the likelihood that the user's body would obstruct the chemiluminescent signal produced as a result of an insult. Moreover, locating a treated area near an edge of the absorbent article may also allow a caregiver to more easily ascertain whether, for example, a bed pad has been insulted, by lifting an edge or corner of the bed covering to check the treated area. A configuration suitable for such situations is shown in FIG. 9F, in which an embodiment of structural element 900 includes a treated area 904 on surface 902 that includes multiple discrete portions 924 that do not overlap, but rather are located away from, target region 926. The individual chemiluminescent components can be disposed in discrete portions 924 as described above for the discrete portions of FIG. 9B, shapes of FIG. 9C, or as otherwise described above.

In some applications, it may be difficult to predict a target region in which a fluid insult can be expected. In the case of a bed pad or pet pad, the location of a fluid insult may vary with the user's relative position, which may shift during sleep or bedrest or other use of the absorbent article, so that a fluid insult may occur at any location relative to the absorbent article. A configuration featuring a pattern consisting of multiple discrete treated portions, which extends over a large area of the surface of structural element 900, as shown in FIG. 9C, for example, may thus be appropriate.

In any of the aforementioned example embodiments, the at least one component (also described herein as chemiluminescent material, first component, component, etc.) may be a luciferin or a luciferase, in accordance with the various species and concentration ranges as detailed herein.

Alternatively, the at least one component may be both a luciferin and a luciferase, in accordance with application techniques disclosed herein for applying two reactive components of a chemiluminescent system to the same substrate material as described above in alternative embodiments, such as the same or overlapping portion(s)/surface(s) of a substrate material (for example, see FIGS. 5D, 5E, and 5G).

In some applications, however, it may be preferable to incorporate both a luciferin and a luciferase into the structural element, but in a manner that avoids the challenges of application to the same or overlapping portions of a substrate material.

As explained in greater detail below, a variety of application techniques may be suitable for producing the various example embodiments of structural element 900 discussed herein. For example, a method of producing a structural element treated with at least one component of a chemiluminescent system, which reacts in the presence of an aqueous system to produce light, for incorporation into an absorbent article, may include applying a formulation to a predetermined area of a first surface of a structural element, wherein the formulation comprises the at least one component, and wherein the at least one component is selected from a luciferin and a luciferase. As noted above, the predetermined area may at least partially overlap one or more target regions in which a fluid insult may be expected during use of an absorbent article into which the structural element is incorporated, and may take any form, as explained above. Certain application techniques, e.g., inkjet printing techniques such as continuous inkjet printing, may allow for increased speed and/or accuracy of application, which may in turn allow configurations of a treated area in which two or more reactive components are applied in close proximity to each other, including overlapping patterns. Thus, in some applications, the treated area may incorporate both a luciferin and a luciferase, such as in a configuration that includes the components respectively applied to non-overlapping portions of the treated area. A configuration exemplifying such an arrangement is shown in FIG. 9G, in which an embodiment of structural element 900 includes a treated area 904 on surface 902 that is similar to that shown in FIG. 9E, but in which discrete portions 928 each consist of non-overlapping segments to which the two components are separately applied. “Non-overlapping” means that at least a minimal distance separates the two segments such that the chemiluminescent components disposed in each are not in fluid communication after application and prior to at least a first fluid insult. More specifically, discrete portions 928 each include two segments 930, which may be treated with one of the components, on either side of a segment 932, which may be treated with the other of the components. In such a configuration, the treated area may overlap a target region (not shown, for clarity). In use, when a fluid insult is present, one or both of the reactive components may be transferred to the other as the fluid wicks, to initiate the light-producing reaction, when in “fluid communication” with the other or both.

In addition to limiting treated areas 928 of FIG. 9G (for example, but not limiting) to an area of surface 902 expected to encounter a fluid insult at a given time, treating adjacent areas 930 and 932 with either of the individual components may allow for substantial savings of raw materials in many of the embodiments described herein by reducing or eliminating a broadly treated area (such as the surface 902 of structural element 900, the material within absorbent core, or other structure in the article) with at least one of the components. The pattern and/or shape of the adjacent treated areas is not limited. A non-limiting example is shown in FIG. 9J in two shapes, the shape of two adjacent and non-overlapping stars and moons on surface 902 of structural element 900. Inner star 940 includes a treated area comprising one component of the chemiluminescent system (e.g., a luciferin or a luciferase). Outer star 942 comprises the other component. Inner moon 941 includes a treated area comprising one component of the chemiluminescent system (e.g., a luciferin or a luciferase). Outer moon 943 comprises the other component. In this illustrative example, the outer star 942 and outer moon 943 are in a discontinuous (i.e., dashed) line, while inner star 940 and inner moon 941 are patterned in a continuous line. Further non-overlapping shapes and/or patterns may be incorporated within or outside of these shapes (not shown). Also, colored inks or pigments may be incorporated in any or all of the shapes to provide for a colored effect in lighted conditions and/or alterations of the observed color emitted by the chemiluminescent reaction (not shown), for example, as printed on an impermeable poly back sheet 900.

Moreover, the coat weight, concentration of the employed component(s), and/or distance between the inner shape 940, 941 and the outer shape 942, 943 can be varied (including overlapping patterns) within the structural element to manipulate the observed light emission intensity and duration of the chemiluminescent reaction. For example, fixed distances relatively close together like the star shapes 940 & 942 will create a faster transfer and availability of each component, assuming all other variables being equal. In comparison, moon shapes 941 & 943 have both close and distant dimensions between them, which will allow for a more sustained reaction and light emission property. The light emission properties can be further altered by varying the distance between the shapes, such as the star shapes 940 & 942 being closer together when placed further from the expected area of insult and further apart when placed nearer to the expected area of insult. Alternatively or conjointly, the coat weight or concentration of one or both of the components within the inner and outer shape treated areas can be varied throughout or in advantageous zones (e.g., concentric regions) on surface 902. Fixed or variable availability (e.g., spatial or concentration) of an excipient having reaction enhancing or inhibiting activity can similarly control light emission duration and/or intensity form the chemiluminescent reaction (not shown). Spatial separation for deferred reaction of the components of the chemiluminescent system can also be achieved by deployment of the components individually in separate and distinct structural elements 900 (e.g., the luciferin printed in star shape 940 on the poly back sheet and the luciferase diffusely throughout the absorbent core fluff pulp) or as shown in FIGS. 9H & 9I.

As noted above, the application of one or both chemiluminescent components may produce a treated area that is not visually distinct from untreated areas. In some embodiments, it may be desirable to have the treated area visually distinct, for example to quality-check the application process, to provide a visual wetness indicator other than chemiluminescence, and so forth. This may be accomplished by various means, one example of which is to also apply a non-chemiluminescent wetness indicator to the first surface. The non-chemiluminescent wetness indicator may be applied to at least partially overlap a target region, but not overlap the treated area, and so forth, as desired. Continuous inkjet printing, optionally in coordination with other application techniques, may be used to produce embodiments that also incorporate a non-chemiluminescent wetness indicator.

In still other embodiments of treated materials or structural elements in accordance with the present disclosure, one or more chemiluminescent components may be provided as discrete quantities in the form of a fluid, a gel, a powder, and so forth, such as to be incorporated into a pre-made absorbent article (for example, to convert such an absorbent article into one that incorporates a chemiluminescent system) or pre-made structural elements therefor. In some of such embodiments, an end user may incorporate one or more chemiluminescent components into a pre-made absorbent article, and so forth.

### Absorbent Articles

As noted above, it will be evident that multiple types of the treated materials in accordance with the present disclosure may be incorporated into an absorbent article. An illustrative example is provided in FIG. 4F, which incorporates treated tissue composition 300 and a layer of indicator particles 400, each of which incorporates a different chemiluminescent component, such that a first chemiluminescent component is transferred to a second chemiluminescent component by an aqueous system in the form of a fluid insult moving through the absorbent article, such as to initiate the light-producing reaction.

As another illustrative example, an embodiment of an absorbent article incorporates absorbent core 502, discussed further herein with respect to FIG. 7, together with a layer of indicator particles 400 arranged between the absorbent core and the back sheet of a diaper, in a manner structurally similar to the configuration shown in FIG. 4F. In such an embodiment in which the absorbent core 502 incorporates a first chemiluminescent component, and the particles 400 are treated with a second chemiluminescent component, a fluid insult moving through the diaper would transfer one chemiluminescent component to the other.

In a more general sense, however, absorbent articles produced in accordance with this disclosure may, but need not, incorporate the various treated materials described herein. Rather, absorbent articles produced in accordance with this disclosure may incorporate a chemiluminescent system by having the chemiluminescent components thereof separately disposed in (or on) a variety of structural elements used in an absorbent article (which may include one or more of the aforementioned treated materials).

In other words, an absorbent article constructed in accordance with this disclosure includes a liquid permeable top sheet, a liquid-impermeable back sheet, an absorbent material (such as fluff pulp and/or synthetic fibers) disposed between the top sheet and the back sheet, and one or more structural elements selected from the group including a liquid permeable tissue sheet and a particle that is formed from or otherwise includes hydrogen bonded cellulose pulp fibers, and a chemiluminescent system adapted to react in the presence of an aqueous system to produce light. In an embodiment, the liquid-permeable tissue sheet comprises fibers selected from cellulose fibers, synthetic fibers, and combinations thereof. The components of the chemiluminescent system, in such an absorbent article, are separately disposed in (or on) the top sheet, the back sheet, the absorbent material, and the structural element(s), in a configuration in which a first chemiluminescent component is transferred to a second chemiluminescent component by an aqueous system moving through the absorbent article.

Thus, the illustrative configurations illustrated in FIGS. 4D, 4E, and 4F are all example embodiments of such an absorbent article.

In some embodiments, one of the chemiluminescent components is disposed within the absorbent material (e.g., fluff pulp, synthetic fibers, SAP, and so forth). In some of such embodiments, another chemiluminescent component is disposed in a liquid permeable tissue sheet, such as a treated tissue composition as discussed above, and such as is shown, for example, in FIG. 4D. In some examples of this configuration, the chemiluminescent system includes a luciferin and a luciferase, with the luciferase disposed within the absorbent material and the luciferin disposed on the tissue sheet. In a particular embodiment, the luciferase is Gaussia luciferase, and the luciferin is coelenterazine. In a particular embodiment, the luciferase is Renilla luciferase, and the luciferin is coelenterazine. In a particular embodiment, the luciferase is Metridia luciferase, and the luciferin is coelenterazine. In a particular embodiment, the absorbent material treated with the chemiluminescent component includes cellulose fibers.

The incorporation of the chemiluminescent system into the absorbent articles discussed herein is generally done by the producer of the absorbent article, and/or the manufacturer(s) of the treated material(s) or structural element(s) integrated into the absorbent article. However, it is within the scope of this disclosure that the end user (or another individual, for example) may apply one or more chemiluminescent components into an absorbent article. For example, in some embodiments in accordance with this aspect of the disclosure, an absorbent article includes a liquid permeable top sheet, a liquid-impermeable back sheet, an absorbent material disposed between the top and back sheets, and a first chemiluminescent component disposed between the top sheet and the back sheet. Such embodiments also include a second chemiluminescent component in a measured quantity suitable for reaction with the first chemiluminescent component, in the presence of an aqueous system, to produce light of a predetermined duration and/or intensity. In such embodiments, which may be provided, for example, as a kit to the end user, the measured quantity may be provided in any suitable form, such as a liquid formulation, a gel, a powder, in particulate form such as the treated dice or strips disclosed herein, encapsulated particles, and so forth, such as to apply to the absorbent article. The absorbent article, in such embodiments, may be configured to allow application of the measured quantity, such as by incorporating a portion that is selectively openable and re-sealable. In one illustrative embodiment, a back sheet includes a flap that may be selectively peeled back in order to allow application of the measured quantity of the second chemiluminescent component to the interior structure of the absorbent article, and then replaced. Further, some embodiments may be configured to allow an existing (e.g., pre-made) absorbent article to be modified to include a chemiluminescent system, such as by an end user or third-party manufacturer such as by applying measured quantities of chemiluminescent components to the absorbent article.

Regardless of the form or method of incorporating the chemiluminescent system, or the components thereof, into an absorbent article, the total quantities may vary considerably depending, for example, on the configuration and use of the absorbent article. As an illustrative and non-limiting example, embodiments such as a baby standard diaper, for example, may include 0.00001-100.0 mg of luciferin and 0.00001-100.0 mg luciferase. In an embodiment, the absorbent article includes 0.00001-100.0 mg, 0.0001-100 mg, 0.001-100 mg, 0.01 mg-100 mg, 0.1-100 mg, or 1-100 mg of luciferin and 0.00001-100.0 mg, 0.0001-100 mg, 0.001-100 mg, 0.01 mg-100 mg, 0.1-100 mg, or 1-100 mg of luciferase, or within any range within any of these ranges, and all other possible subranges.

Some of such embodiments include coelenterazine in a total quantity ranging from 0.0001-20.0 mg and luciferase (Gaussia luciferase, Renilla luciferase, Metridia luciferase, or combinations thereof) in a total quantity ranging from 0.00003-20.0 mg. Some of such embodiments include coelenterazine in a total quantity ranging from 0.01-100.0 mg and luciferase (Gaussia luciferase, Renilla luciferase, Metridia luciferase, or combinations thereof) in a total quantity ranging from 0.2-40.0 mg. Of course, baby diapers may include lesser and greater amounts, and other absorbent articles may also include different amounts and/or ranges of the respective components.

### Treatment Formulations

Formulations useful for applying a chemiluminescent system to a substrate material, for example in the production of the treated materials and/or absorbent articles disclosed herein, include at least one component of the chemiluminescent system, selected from a luciferin and a luciferase, in a liquid carrier. Depending on factors such as its solubility in the liquid carrier, the reactive component may be dispersed in the liquid carrier, and/or dissolved, and so forth. The term “formulation,” as used herein, thus encompasses mixtures (e.g., dispersions, suspensions, and so forth) as well as solutions. For example, in an illustrative embodiment, the liquid carrier is a suitable solvent (e.g., ethanol) in which luciferin is dissolved. In an illustrative embodiment, the liquid carrier is water in which luciferase is dissolved; however, wherein the solvent is ethanol (such as in the form of a ground powder), the luciferase is dispersed.

Formulations in accordance with the present disclosure include one-component formulations—that is, incorporating either a luciferin or a luciferase—and two-component formulations—that is, incorporating both a luciferin and a luciferase.

As noted above, the chemiluminescent system, and specifically the components thereof, react in the present of water to produce light. One challenge in the incorporation of the reactive components into an absorbent article, and/or a material or structural element for an absorbent article, is doing so in a manner that does not prematurely initiate the reaction, such as during production. Although one manner of addressing this challenge is by providing the components in separate materials or structural elements, and/or in different locations in the absorbent article, in some applications it may be suitable to incorporate both components in the same substrate material. In such applications, the challenge therefore may become one of producing the substrate material in a manner that does not prematurely initiate the chemiluminescence. One way of addressing this challenge is in providing a two-component formulation in which the components do not react with each other. For example, in an illustrative embodiment of a two-component formulation, the liquid carrier is a suitable non-aqueous solvent (e.g., ethanol) in which luciferin is dissolved and in which the luciferase is dispersed.

A variety of solvents are suitable for use in, or as, the liquid carrier for either one-component or two-component formulations. Suitable solvents for luciferin, for example, include ethanol, isopropanol, n-butanol, isobutanol, ethyl acetate, methyl acetate, isopropyl acetate, acetone, pentanone, methyl ethyl ketone, n-butyl acetate. Combinations of the aforementioned solvents may also be used, illustrative examples of which include ethanol and isopropyl acetate, ethanol and acetone, pentanone and ethanol, and so forth. The aforementioned solvents and combinations are also suitable media in which luciferase may be dispersed. The choice of solvent may be determined by several factors, including solubility of the particular luciferin(s) in the solvent, whether other substances (e.g., luciferase, a binder, a porous transfer agent, a viscosity adjusting agent, a photoluminescent compound, a pH buffer, and so forth) will be included in the formulation, process considerations such as application method of the formulation, the substrate material(s) to which the formulation will be applied, and so forth.

The concentration of the chemiluminescent component(s) in the formulations is not particularly limited in the general sense, except by the solubility limit of a solvent of the liquid carrier, and may be as suitable for the particular application.

However, it was found that even in some situations in which a chemiluminescent component is not particularly soluble in a solvent, the solubility thereof can be facilitated through the use of an excipient—that is, a compound that functions to facilitate solubility of the chemiluminescent component in the solvent. One example of this is in the case of some luciferins, such as coelenterazine, which are not very soluble in water. However, for various reasons (e.g., handling requirements, recycle/recovery/disposal costs, raw material cost, capabilities of existing manufacturing equipment, etc.), water may be a more desirable solvent than, for example, the aforementioned organic solvents. Suitable excipients for coelenterazine generally include polar protic solvents other than water, including some of the aforementioned suitable organic solvents for luciferins (e.g., ethanol, butanol, propanol, isopropanol, pentanone) as well as other materials, such as hydroxypropyl-β-cyclodextrin (HPBC), and so forth, and combinations thereof. In some embodiments, the use of a solvent as an excipient in a partially aqueous formulation may be a suitable approach to decrease the amount otherwise used in a formulation.

Thus, in some embodiments, a partially aqueous formulation includes a luciferin, and a solvent to dissolve the luciferin that includes water and an excipient that facilitates solubility of the luciferin in water. In an embodiment, the partially aqueous formulation includes a luciferin, a solvent to dissolve the luciferin that comprises 40-99 weight percent water and 1-60 weight percent of an excipient adapted to facilitate the solubility of the luciferin in water, and, optionally, a binder adapted to bind luciferin to the substrate material. In an embodiment, the partially aqueous formulation includes a solvent that comprises 40-99, 50-99, 60-99, 70-99, 80-99, 90-99, or 95-99 weight percent water, or within any range within any of these ranges, and all other possible subranges. In an embodiment, the partially aqueous formulation includes 1-60, 5-60, 10-60, 20-60, 30-60, 40-60, 50-60, or more weight percent of an excipient, or within any range within any of these ranges, and all other possible subranges.

In some of such embodiments, the excipient is one or more of hydroxypropyl-β-cyclodextrin, ethanol, butanol, propanol, isopropanol, and pentanone. The effect of some excipients may be additive, in terms of contributing to the solubility of a luciferin. In such embodiments, the solvent is generally at least 40 weight percent water, and the concentration of the excipient may be determined by the desired concentration of the luciferin to be dissolved in the solvent. For example, in embodiments in which HPBC is used as an excipient, a concentration of about 45-50 mM of HPBC can facilitate dissolution of coelenterazine in water up to about a 3.7 mM concentration (this correlates to about 1.57 g coelenterazine per liter of water).

Accordingly, in some embodiments of a formulation in accordance with this disclosure, the liquid carrier includes a solvent in which a luciferin is dissolved, with the composition of the formulation ranging between 40 to 99 weight percent of the solvent, and 0.01 to 20 weight percent luciferin. In an embodiment, the formulation includes 40-80 weight percent, 45-85 weight percent, 50-90 weight, 60-95 weight percent, or 70-99 weight solvent, or within any range within any of these ranges, and all other possible subranges. In an embodiment, the formulation includes 0.01 to 20, 0.1 to 10, 0.1 to 15, 1 to 15, 5 to 15, or 5 to 20 weight percent luciferin, or within any range within any of these ranges, and all other possible subranges. In some of such embodiments, the solvent is selected from ethanol, isopropanol, n-butanol, isobutanol, ethyl acetate, methyl acetate, isopropyl acetate, acetone, pentanone, methyl ethyl ketone, n-butyl acetate, and combinations thereof. In examples of such an embodiment, the solvent includes ethanol. In some non-limiting examples of such an embodiment, the solvent is ethanol, and the formulation includes between 80-99 weight percent ethanol. In some non-limiting examples of such an embodiment, the solvent is ethanol, and the formulation includes between 45-50 weight percent ethanol. In some of the aforementioned embodiments, the luciferin is selected from coelenterazine, a coelenterazine analog, dinoflagellate luciferin, bacterial luciferin, fungal luciferin, firefly luciferin, vargulin, furimazine, and combinations thereof. In some non-limiting examples of such an embodiment, the luciferin is coelenterazine, and the formulation includes 0.1 to 0.3 weight percent luciferin. In some non-limiting examples of such an embodiment, the luciferin is coelenterazine, and the formulation includes 0.2 to 0.5 weight percent luciferin. In some non-limiting examples of such an embodiment, the luciferin is coelenterazine, and the formulation includes 0.5 to 0.9 weight percent luciferin. In some non-limiting examples of such an embodiment, the luciferin is coelenterazine, and the formulation includes 0.9 to 2.0 weight percent luciferin. In some non-limiting examples of such an embodiment, the luciferin is coelenterazine, and the formulation includes 2.0 to 10 weight percent luciferin. In some non-limiting examples of such an embodiment, the luciferin is coelenterazine, and the formulation includes 10 to 20 weight percent luciferin.

Luciferin-containing formulations may further include luciferase, in embodiments in which the liquid carrier is non-aqueous (and thus in which the chemiluminescent components will not react). Accordingly, in some embodiments of a formulation in accordance with this disclosure, a non-aqueous liquid carrier includes a solvent in which a luciferin is dissolved, with the composition of the formulation ranging between 40 to 99 weight percent of the solvent, between 0.01 to 20 weight percent luciferin, and between 0.01 to 20 weight percent luciferase. In such embodiments, the solvent and luciferin may be as indicated above. In some of such embodiments, the luciferase is selected from Gaussia luciferase, Renilla luciferase, Metridia luciferase, Oplophorus luciferase, dinoflagellate luciferase, copepod luciferase, and firefly luciferase. In some non-limiting examples of such an embodiment, the luciferase is Gaussia luciferase, Renilla luciferase, Metridia luciferase, and combinations thereof, and the formulation includes 0.05 to 0.2 weight percent luciferase. In some non-limiting examples of such an embodiment, the luciferase is Gaussia luciferase, Renilla luciferase, Metridia luciferase, and/or combinations thereof, and the formulation includes 0.2 to 0.6 weight percent luciferase. In some non-limiting examples of such an embodiment, the luciferase is Gaussia luciferase, Renilla luciferase, Metridia luciferase, and/or combinations thereof, and the formulation includes 0.1 to 1.0 weight percent luciferase. In some non-limiting examples of such an embodiment, the luciferase is Gaussia luciferase, Renilla luciferase, Metridia luciferase, and/or combinations thereof, and the formulation includes 1.0 to 10.0 weight percent luciferase. In some non-limiting examples of such an embodiment, the luciferase is Gaussia luciferase, Renilla luciferase, Metridia luciferase, and/or combinations thereof, and the formulation includes 10.0 to 20.0 weight percent luciferase.

Embodiments in which the liquid carrier/solvent is aqueous or partially aqueous are generally one-component formulations, as the chemiluminescent components react in the presence of water to produce light.

Formulations in accordance with this disclosure, such as any of those discussed above, may include one or more of a variety of other substances in addition to the liquid carrier/solvent and chemiluminescent component(s). As noted above, one example is a binder, for example to assist retention of the chemiluminescent component(s) on the substrate material to which the formulation will be applied, as described above with regard to the treated materials produced in accordance with this disclosure. Suitable binders are discussed above, and include ethyl cellulose, methyl cellulose, nitrocellulose, and polyurethane. Although not required to all embodiments, binders may act as a water barrier and/or time-release agent, as discussed above. Another example is a porous transfer agent, also as described above with regard to the various embodiments of treated materials. A porous transfer agent incorporated into a formulation as disclosed herein may provide a benefit after application of the formulation to the substrate material. In particular, a porous transfer agent may facilitate transfer of the chemiluminescent component(s), and/or an aqueous system, relative to a substrate material to which the formulation is applied, upon later contact of the treated substrate material with an aqueous system. As noted above, a porous transfer agent may thus ameliorate the tendency of some binders to reduce availability of the chemiluminescent components when an aqueous system is present. Suitable porous transfer agents are also discussed above, and include starch, amorphous silica, clay minerals, cellulose pulp fiber, cotton fiber, and synthetic polymer fiber. In an embodiment, the formulation includes a porous transfer agent in a range of about 0.01 weight percent to about 52 weight percent. In an embodiment, the formulation includes a porous transfer agent in a range of about 0.01 to about 5 weight percent, of about 0.1 to about 10 weight percent, of about 1 to about 20 weight percent, of about 5 to about 40 weight percent, or of about 10 to about 52 weight percent, or within any range within any of these ranges, and all other possible subranges.

Yet another example of an optional additive substance is a viscosity adjusting agent, which may be used to adjust the viscosity of the formulation to a desired level, such as a viscosity suitable for application of the formulation to a substrate material by any of a variety of methods (e.g., streaming, printing, coating, and so forth). Although technically speaking, any soluble or non-soluble additive to a solvent may function to alter the fluid dynamic properties of the solvent, the term “viscosity adjusting agent” refers herein to substances which impart a non-negligible change to the viscosity of a formulation. Suitable viscosity adjusting agents thus include substances that may also be suitable binders, such as ethyl cellulose, methyl cellulose, nitrocellulose, and polyurethane, substances that may also be suitable porous media, and so forth.

The inclusion of one or more of the aforementioned additives, the selection thereof, the concentration thereof, and so forth, may be determined by several factors, such as the intended application method for the formulation, the substrate material(s) to which the formulation will be applied, interaction between and among other substances in the formulation, and so forth.

For example, formulations that include a binder may include high levels thereof, for example if the substrate material would otherwise not retain the chemiluminescent component(s) well, or at all. On the other hand, it was found that even low levels of binder enhanced retention in some applications. Although this is not intended to be limiting, in general it was found that a suitable concentration for a binder in formulations in accordance with this disclosure ranged between 0.01 to 15 weight percent of the formulation. For example, in some of such embodiments, the binder concentration ranges between about 8.0 to about 12.5 weight percent. In some of such embodiments, the binder concentration ranges between about 0.01 and 1.2 weight percent. In some of such embodiments, the binder concentration ranges between about 1.2 and 8.0 weight percent. In an embodiment, the binder concentration is in a range of about 0.01 to about 1 weight percent, of about 0.1 to about 5 weight percent, of about 1 to about 10 weight percent, or about 1 to about 15 weight percent, or within any range within any of these ranges, and all other possible subranges.

Similarly, formulations that include a viscosity adjusting agent are not particularly limited in the amount of agent that is used, but in general it was found that a suitable concentration for a viscosity adjusting agent in formulations in accordance with this disclosure ranged between 0.01 to 30 weight percent of the formulation. In an embodiment, the viscosity adjusting agent has a concentration ranging from 0.01 to 30, 0.1 to 10, 1 to 15, 5 to 30 weight percent of the formulation, or within any range within any of these ranges, and all other possible subranges. One factor that may determine suitability is the application method for the formulation (discussed below).

As noted above, porous transfer agents may ameliorate the tendency of some binders to resist release of a chemiluminescent component from the substrate material in which it is incorporated, upon contact with an aqueous system. Even in the absence of such a binder, however, a porous transfer agent may facilitate the availability of the chemiluminescent component when an aqueous system is present. Even a small amount may be beneficial, in such circumstances. On the other hand, as many porous transfer agents are suspended or dispersed in the formulation rather than dissolved, high amounts of porous transfer agents may be used to produce a thicker, viscous formulation, for example to limit mobilization or flow of the formulation relative to the substrate material after it has been applied. This may be useful, for example, in applications in which it is desired to have the chemiluminescent component(s) localized or even limited to one or more specific areas on the substrate material, applications in which the substrate material may have less ability to limit the free flow of the formulation once applied (for example, a poly back sheet), and so forth. It may also be useful to reduce post-application drying time. In general, it was found that a suitable concentration for a porous transfer agent in formulations in accordance with this disclosure ranged between 0.01 to 52 weight percent of the formulation, depending on the application. For example, in some of such embodiments, the porous transfer agent concentration ranges between about 3.0 to about 6.0 weight percent. In some of such embodiments, the porous transfer agent concentration ranges between about 10 to about 25 weight percent. In some of such embodiments, the porous transfer agent concentration ranges between about 40 to about 52 weight percent.

Although several particular embodiments are discussed more thoroughly in the “Examples” section herein, the following summarizes various illustrative and non-exclusive example formulations in accordance with the present disclosure.

In a non-limiting example, the component is a luciferin, wherein the luciferin comprises coelenterazine, and wherein the liquid carrier includes a solvent in which the coelenterazine is dissolved and that comprises ethanol; and wherein the formulation comprises: 40-90 weight percent ethanol; 0.1-5.0 weight percent coelenterazine; 0.01-15 weight percent binder; and 9-52 weight percent porous transfer agent.

In a first, simple example “one-component” formulation, coelenterazine (“CTZ”) is dissolved in ethanol. A sample composition of such a formulation is 89 weight percent ethanol, and 11.0 weight percent raw CTZ (of about 50% purity). The formulation was found to be suitable for effective application of CTZ to a cellulosic tissue sheet. When incorporated into an absorbent article along with luciferase-treated fluff pulp, the chemiluminescence generated upon testing the structure was visible in a dark room without light, indicating that CTZ of comparatively low purity could be used in such applications.

Variants of the aforementioned formulation also included one or more binders dissolved in the solution. A sample composition, also using raw CTZ at about 50% purity, included 87.39 weight percent ethanol, 0.88 weight percent ethyl cellulose (a binder), 0.33 weight percent polyurethane (also a binder), 0.40 weight percent corn starch, and 11.0 weight percent of raw CTZ. The formulation also effectively applied CTZ to a tissue sheet. When incorporated into an absorbent article and tested as described above, the chemiluminescence was similarly visible, with some differences in observed terms of intensity after consecutive insults. Specifically, in this application, the presence of binders and the starch releasing agent in the formulation correlated to higher light production after fewer insults and less light production after more insults. Adding PVPA as a releasing agent or in place of a binder results in higher light production after fewer insults and less light production after more insults (see FIG. 15A). In contrast, utilizing a treated fluff pulp containing 2% alum (see FIG. 16) or adding hydroxypropyl-β-cyclodextrin in the luciferin application formulation acts as a binder to limit the availability of the luciferin. Both delay light production until after more insults.

In an non-limiting example, the formulation is a two-component formulation including wherein the luciferin comprises coelenterazine, the luciferase comprises Gaussia luciferase, Renilla luciferase, and/or Metridia luciferase, and the solvent comprises ethanol; and wherein the formulation comprises: 50-99 weight percent ethanol; 0.1-5.0 weight percent coelenterazine; 0.1-5.0 total weight percent luciferase; optionally, 0.01-30 total weight percent of one or more of: a binder adapted to retain the at least one component on a substrate material to which the formulation is applied; and a viscosity adjusting agent; and optionally, 0.1-10 weight percent porous transfer agent.

In a first, simple example “two-component” formulation, CTZ and ethyl cellulose are dissolved in ethanol, and Gaussia luciferase (“GLuc”) is dispersed in the solution. A sample composition of such a formulation is 89.8 weight percent ethanol, 8.6 weight percent ethyl cellulose, CTZ in a concentration less than 2.0 weight percent, and GLuc in a concentration less than 2.0 weight percent. The formulation was found to be suitable for effective application of both chemiluminescent components to a poly back sheet suitable for a diaper and is of a suitable viscosity for application via line-streaming.

Variants of the aforementioned formulation included a porous transfer agent dispersed in the solution. A sample composition, using corn starch as porous transfer media, is 82.5 weight percent ethanol, 8.8 weight percent ethyl cellulose, 3.3 weight percent polyurethane (another binder), CTZ in a concentration less than 1.0 weight percent, GLuc in a concentration less than 2.0 weight percent, and 4.0 weight percent corn starch. Another sample composition substitutes synthetic amorphous silica for the corn starch, but is otherwise the same. Both of these formulations were found to also be suitable for effective application of both chemiluminescent components to a poly back sheet suitable for a diaper. However, when tested, the chemiluminescence exhibited by the back sheet treated with formulations that included porous media was much stronger than that exhibited by the back sheet treated with the formulation that did not include porous media. Such formulations are also of suitable viscosity for application via line-streaming.

Among other compositional variations in the formulations summarized herein (e.g., inclusion of a binder, releasing agent, porous media, or other additive materials, and the concentration ranges thereof) is that the “two-component” formulations may instead include only one chemiluminescent component, for example in applications in which it is desired to produce a material or article treated with only one chemiluminescent component, such as in absorbent article configurations in which the chemiluminescent components are disposed separately within the structure of the absorbent article (such as in different materials and/or structural elements of the absorbent article). The converse—that a one-component formulation may be modified, consistent with this disclosure, to include more than one chemiluminescent component, such as both a luciferin and a luciferase—is also true, at least in embodiments in which the solvent or liquid carrier does not prematurely initiate the reaction between the chemiluminescent components or in dry formulation applications. For example, the CTZ-in-ethanol formulation described above could also include luciferase.

An example formulation that illustrates the breadth of the range of an additive material in a formulation is one that includes a comparatively high content porous transfer agent relative to those above, with a composition of 47.10 weight percent ethanol, 1.49 weight percent ethyl cellulose, polyurethane in a concentration less than 1.0 weight percent, CTZ in a concentration less than 0.50 weight percent, and 50.67 weight percent corn starch. The formulation was found to be flowable even with the high content of porous media, and was also found to be suitable for effective application of CTZ to a poly back sheet for a diaper. Once applied, the formulation was found to substantially remain in place on the substrate material and resisted migration/flow to other areas, and was found to exhibit effective bioluminescence when assembled into a diaper-like structure with an absorbent material that included luciferase, when subjected to insult testing. Although this example formulation is a “one-component” formulation, variants could also include luciferase, as the solvent would not trigger the light-producing reaction between the two chemiluminescent components.

In an example “one-component” formulation, coelenterazine of up to 3.7 mM concentration (about 1.57 g per liter) is dissolved in an aqueous solution of 45-50 mM hydroxypropyl-β-cyclodextrin. In another example, coelenterazine of desired concentration up to about 11 weight percent is dissolved in 1-30% ethanol or isopropanol (or a combination thereof). Variants of these examples also include up to 15 weight percent of a binder, and/or a porous transfer agent. Such partially aqueous luciferin formulations would thus be suitable for application of the luciferin for a variety of substrate materials discussed herein, for example absorbent materials such as fluff pulp, synthetic fibers, or combinations thereof. In another example, solvent soluble excipient, such as poly(l-vinylpyrrolidone-co-vinyl acetate (PVPA)), etc. can be added to a partial solvent system of 1-30% ethanol or isopropanol. The solvent soluble PVPA, for example, has a concentration of 0.2% in the partial solvent system. An addition of 0.2% PVPA has a dramatic effect on luciferin availability after fewer insults of an aqueous solution (see FIG. 15A).

In the “one-component” formulation above, in accordance with the principles and concepts disclosed herein, an artisan will be able to determine an appropriate combination of factors for any configuration of an absorbent article with no more than reasonable experimentation. In embodiments in which the reactive component is the luciferin coelenterazine, the treated tissue composition may comprise from 0.00002 to 20 weight percent coelenterazine. For example, in one such embodiment, the treated tissue composition comprises from 1 to 6 weight percent coelenterazine.

### Treatment Methods

In general, the formulations and variants thereof in accordance with one aspect of this disclosure may be applied to or incorporated into a substrate material using various methods, such as those disclosed in the aforementioned U.S. patent application Ser. No. 14/516,255, which describes several methods for incorporating a chemiluminescent system into fluff pulp, such as coating, rinsing, dipping, or spraying one or more non-aqueous solutions of the respective chemiluminescent component(s) onto a fluff pulp sheet prior to an air-laid process.

In one aspect of this disclosure, additional methods and techniques related to treating a substrate material with one or more reactive components of a chemiluminescent system are provided. In such embodiments, a substrate material is treated with one or both reactive components of a bioluminescent system—that is, a luciferin and a luciferase. Such methods may be used in addition to or as an alternative to standard methods and/or those described in the aforementioned '255 application, either to fluff pulp and/or to other substrate materials and/or structural elements, such as those suitable for use in an absorbent article.

As one illustrative and non-exclusive example of such a method in accordance with this disclosure, a method of producing a fluff pulp composition may be performed during an air-laid process, such as when a fluff pulp sheet is fiberized in a hammermill. Such a method includes fiberizing a sheet of fluff pulp fibers to produce a dispersion of individualized fluff pulp fibers in air, and spraying a formulation of at least one component of a chemiluminescent system into the dispersion. In such a method, the spraying step is configured to deposit an amount of the chemiluminescent component(s) on the individualized fluff pulp fibers corresponding to a concentration of 0.0003 to 10 weight percent of the component. The formulation in such a method is not particularly limited, and for example may take the form of any of those described herein. The spraying step may take place at various locations in the process. For example, in some embodiments, the fiberizing is performed in the chamber of a hammermill, and the formulation is sprayed into the chamber of the hammermill. In some embodiments in which the fiberizing is performed in the chamber of a hammermill the dispersion of fibers is then air conveyed from the hammermill, and the formulation is sprayed into the dispersion while and/or after it is air conveyed from the chamber.

Another illustrative and non-exclusive example of a method of producing a treated fluff pulp composition includes separately applying a non-aqueous solution that includes a luciferin, and an aqueous solution that includes a luciferase, to portions of a fluff pulp sheet. In some embodiments of this method, the portions are non-overlapping, such as on opposing surfaces of the fluff pulp sheet. Optionally, the non-overlapping portions may be on the same surface of the fluff pulp sheet. In one example, by substantially drying an aqueous application of the luciferase component prior to or after the application of the luciferin component, the separately applied components may be overlapping (where substantially drying means that there is insufficient water to allow for more than 5% of one or both components in the treated area to be consumed in a chemiluminescent reaction without further addition of water).

Of course, several treated materials and structural elements in addition to treated fluff pulp are described above, examples of which include treated tissue compositions, indicator particles of various sizes and shapes, absorbent cores and similar articles that include absorbent materials such as SAP and synthetic and/or cellulose (e.g., fluff pulp) fibers, other structural elements (a liquid permeable top sheet, a liquid permeable back sheet, and so forth) and parts thereof, and so forth.

Several formulations for producing the treated materials, or otherwise applying one or more chemiluminescent components to a substrate material, are also described above.

Developing a suitable application method of a treatment formulation to a substrate material or structural element can face several challenges, some of which are noted above. For example, one challenge may be incorporating the reactive components into a material and/or an absorbent article in a manner that does not prematurely initiate the light-producing reaction, such as during production or storage, but only during use of an absorbent article. One challenge may be ensuring retention of the chemiluminescent component(s) on the substrate material after application. A related challenge may be limiting mobility of the chemiluminescent component(s) relative to the substrate material after application.

As explained above, some challenges may be addressed by the inclusion of one or more additives into the formulation, such as a binder, for example to enhance retention, and/or porous media, for example to limit mobility, and so forth. Some challenges may be addressed by separately disposing the reactive components in an absorbent article—such as on two or more separate materials or structural elements of the absorbent article—so that one is transferred to the other during use, such as by an aqueous system moving through the absorbent article.

There may be additional challenges, such as in scaling up an application method from lab and/or pilot scale to commercial scale. Some challenges are presented by existing equipment configuration, for example in integrating an application method into existing machinery, such as tissue coating or printing machinery. Some challenges are presented by a desire to use a single application method for a variety of different substrate materials or structural elements. There is also the continuous goal of achieving better process efficiency. These and other challenges may (alternatively or additionally) be addressed in terms of the application method or technique.

As one example, it is often desired to increase the overall speed of a production method. However, applying a formulation to a substrate material generally requires consideration of subsequent removal of the liquid carrier (e.g., water, an organic solvent, etc.) from the material, which is often done with a heating or drying step.

Many materials suitable for incorporation into an absorbent article have the capacity to retain a threshold level of bound water after any free water is removed. This capacity, in cellulosic materials, is referred to as “fiber saturation point,” denoting the point at which only water bound in the cell walls remains (“free water” being any other moisture that is not so bound). The fiber saturation point of fluff pulp can vary considerably, depending on factors such as composition, species, and so forth, but generally ranges between about 15-25 weight percent. The threshold level of moisture for synthetic materials is generally lower, for example a poly sheet will generally have a threshold level of moisture of around 5 weight percent, but this may be as high as 10 weight percent.

It was found that this characteristic could be exploited, in one method of applying a chemiluminescent component to a substrate material, to reduce or eliminate the need for a drying step subsequent to application. Accordingly, in some embodiments, a method of applying a luciferase to a substrate material includes applying a formulation that includes a luciferase dispersed in an aqueous liquid to a surface of the substrate material, such that the moisture level of the substrate material is not increased above its threshold level of moisture.

In certain embodiments, the luciferase is selected from the group consisting of Gaussia luciferase, Renilla luciferase, Metridia luciferase, Oplophorus luciferase, dinoflagellate luciferase, copepod luciferase, and firefly luciferase.

The luciferase formulation may have any desired concentration within a suitable range, which considers factors such as the initial moisture content of the substrate material, the threshold moisture content of the substrate material, the amount of the formulation applied to the substrate material, the desired concentration of the luciferase on the substrate material, and so forth. A representative concentration range of the luciferase on the substrate material, for such a method, is between 0.01-20 mg per gram of substrate material. To achieve this, a concentration range of the luciferase in the formulation, in some embodiments, is between about 5.0-30 weight percent.

In some embodiments, the substrate material has a threshold level of moisture of up to 25 weight percent. In some embodiments, the substrate material is a fluff pulp sheet having a threshold level of moisture (or fiber saturation point) of at least 15 weight percent. In some of such embodiments, the luciferase formulation is applied to the surface at a rate that increases the moisture level of the fluff pulp sheet by less than 10 weight percent. In some embodiments, the substrate material is a poly sheet having a threshold level of moisture of up to 10 weight percent.

The following illustrative example considers a sheet of fluff pulp having a moisture content of 7.5% at ambient conditions and a fiber saturation point of about 15%. The following table shows an illustrative range of application levels of a 15 weight percent aqueous luciferase formulation to achieve a final pulp moisture content less than the fiber saturation point. A fluff pulp sheet treated in this manner may reduce or eliminate the need for a post-application drying step, due to the lack of free water in the treated material.

TABLE 2
Example Final Pulp Moisture Levels Using Different Application Levels
of a 15% Luciferase Formulation

| | Luciferase | Raw | | | |
|---|---|---|---|---|---|
| Luciferase | solution | luciferase | Pure | Net pulp | |
| solution | on | application, | luciferase, | moisture | Final Pulp |
| concentration | pulp, % | mg/g pulp | mg/diaper | increase, % | Moisture, % |
| 15.0% | 6.3% | 10 | 10.72 | 5.7% | 13.2% |
| | 4.5% | 7 | 7.50 | 4.0% | 11.5% |
| | 3.2% | 5 | 5.36 | 2.8% | 10.3% |
| | 2.0% | 3 | 3.22 | 1.7% | 9.2% |

Similar calculations can be performed for other concentrations of a luciferase formulation, such as to achieve a desired concentration of luciferase in a substrate material without exceeding the threshold level of moisture of the material.

Other application methods in accordance with the present disclosure apply two reactive components of a chemiluminescent system to the same substrate material, such as the same or overlapping portion(s)/surface(s) of a substrate material. As noted above, in many applications, it is preferable to dispose the reactive components of a chemiluminescent system in different or separate materials or structural elements of an absorbent article—such as luciferase in an absorbent material (e.g., fluff pulp, synthetic fibers, SAP, and so forth) and luciferin in another structural element (e.g., a treated tissue composition, poly back sheet, flakes, strips, etc.). Such a configuration may better assure that the chemiluminescent components do not inadvertently react prior to use, such as during production, transport, or storage, such as due to ambient moisture or humidity.

Regardless, in some circumstances it may be desired to incorporate more than one reactive component (e.g., both luciferin and luciferase) into the same material or structural element. As described above, this may be done through the use of a non-aqueous formulation that incorporates both luciferin and luciferase. However, it was found that this may also be done using an application method in which luciferase and luciferin are separately applied to the same substrate material—in particular, to the same area or surface of the substrate material. Such a method also exploits the capacity of a substrate material to bind moisture up to a threshold level, such that there is no or insufficient free water available to initiate the light-producing reaction.

Accordingly, in some embodiments, a method of applying a chemiluminescent system to a substrate material includes a luciferase treatment step, in which an area on a surface of the substrate is treated with a luciferase formulation that includes a luciferase dissolved in an aqueous liquid, and a luciferin treatment step, in which said area is treated with a luciferin formulation that includes a luciferin dissolved in a non-aqueous solvent. In such a method, the luciferase treatment step does not increase the moisture content of the substrate material above the threshold level of moisture. As such, the moisture content would not exceed the threshold level of moisture for the substrate material, and thus no free water would be available to initiate the reaction of the chemiluminescent components.

In certain embodiments, the luciferin is selected from the group consisting of coelenterazine, a coelenterazine analog, dinoflagellate luciferin, bacterial luciferin, fungal luciferin, firefly luciferin, vargulin, and furimazine. In certain embodiments, the luciferase is selected from the group consisting of Gaussia luciferase, Renilla luciferase, Metridia luciferase, Oplophorus luciferase, dinoflagellate luciferase, copepod luciferase, and firefly luciferase.

In such a method, the luciferin formulation includes a non-aqueous solvent, which will not initiate a reaction between the chemiluminescent components—rather, the aqueous luciferase formulation is the more significant consideration in terms of moisture level. As before, the luciferase formulation may have any desired concentration within a range suitable considering such factors as the initial moisture content of the substrate material, the threshold moisture content of the substrate material, the amount of the formulation applied to the substrate material, the concentration of luciferin on the substrate material, and so forth. Such a range is generally between about 5.0-30 weight percent. The illustrative example formulation application levels for a 15 weight percent luciferase formulation displayed in Table 2 are applicable to this method as well, and similar calculations can be performed for other concentrations of a luciferase formulation, such as to achieve a desired concentration of luciferase in a substrate material without exceeding the threshold level of moisture of the material.

The aforementioned application methods, which exploit the ability of a substrate material to bind a certain amount of water, offer one approach to reducing or avoiding the need for a subsequent step (such as a drying step) of removing a liquid carrier. However, an alternative application method in accordance with the present disclosure may be suitable for substrate materials having a lower threshold moisture level, such as poly back sheets or similar structural elements that incorporate synthetic materials.

Such a method produces a liquid-impermeable back sheet structure that is treated with at least one component of a chemiluminescent system. In an illustrative embodiment of such a method, a formulation is applied to a surface of a liquid-impermeable back sheet, with the formulation including luciferin and/or luciferase, a liquid carrier, and a binder adapted to retain the chemiluminescent component(s) to the back sheet. At least some of the liquid carrier is then removed from the back sheet.

The formulation in such a method may be among those described herein. One example formulation described above includes luciferin as the chemiluminescent component, dissolved in ethanol, with the composition including 40 to 90 weight percent ethanol, 0.1 to 5.0 weight percent luciferin, 0.01 to 15 weight percent binder, and 9 to 52 weight percent porous transfer agent. As noted above, materials used in or as a liquid-impermeable back sheet, such as synthetic materials, may provide certain challenges such as comparatively low retention of a chemiluminescent component applied thereto, migration or flow of the formulation relative to the material after it has been applied, and so forth. The incorporation of a binder may address the retention issue, and, as described above, a higher amount of porous media in a formulation may address the migration issue. Accordingly, in some embodiments of such a method, the formulation includes a reduced amount of liquid carrier and an increased amount of porous media. A particular, non-limiting example of such a formulation is described above, as including 47.10 weight percent ethanol, 1.49 weight percent ethyl cellulose, polyurethane in a concentration of less than 1.0 weight percent, CTZ in a concentration less than 0.5 weight percent, and 50.67 weight percent corn starch. Variants of this method may, however, include all manner of variations to the formulation according to the principles discussed above, including varying the amount of binder and/or porous media to impart a desired viscosity to the formulation, all of which are considered to be within the scope of this disclosure. In some embodiments of the method, the viscosity of the formulation is suitable for application to the back sheet by streaming, which refers to a process in which a flow of formulation is applied to a surface by means of a nozzle. A suitable viscosity for a streaming application may vary depending on nozzle design and configuration (e.g., the proximity of the nozzle to the surface, etc.). In some embodiments, any viscosity at which the formulation remains flowable is suitable.

As noted above, the method includes subsequent removal of at least some of the liquid carrier from the back sheet. In some embodiments of the method, this removal step includes heat treatment of the surface of the back sheet treated with the formulation. However, in some embodiments, a removal step includes contacting the back sheet with an absorbent material adapted to wick the liquid carrier from the surface of the back sheet. In some of such embodiments, the absorbent material is in the form of an absorbent core that includes absorbent fibers and/or SAP. In some of such embodiments, the absorbent material is treated with luciferase.

In a non-limiting example of such an application method that uses a wicking technique to remove the liquid carrier, the example luciferin-in-ethanol formulation described above was first streamed onto the surface of a poly back sheet. Immediately after application, a diaper core assembly formed from SAP dispersed in luciferase-treated pulp was laid down over the treated area. The diaper core assembly wicked the excess solvent (ethanol, in this case) more quickly than air-drying.

As such, the above-described wicking technique may be integrated into a diaper manufacturing process, in some cases reducing or even eliminating the need for a post-application drying step prior to assembling a diaper structure using the treated back sheet. Moreover, as noted above, if another chemiluminescent component is disposed in the wicking material (such as the diaper core assembly) or elsewhere in the diaper structure, the result is a diaper that incorporates two reactive chemiluminescent components, disposed separately within the diaper structure, such that one is transferred to the other upon receiving a liquid insult.

Variations to this method may include alternative or additional approaches to one or more of the aforementioned challenges. For example, some embodiments of the method may include, subsequent to applying the formulation to the surface of the back sheet, applying a coating over the treated surface, wherein the coating is water soluble or water permeable. In one example, a solvent soluble coating agent, such as ethyl cellulose, may be sprayed and dried on top of the formulation layer already deposited on the back sheet, to form a thin, water-permeable coating of ethyl cellulose. In another example, an aqueous soluble coating agent, such as carboxymethyl cellulose (CMC), may be sprayed and dried on top of the formulation layer already deposited on the back sheet, to form a water soluble film of CMC. Other example coating (i.e., encapsulating) materials include sugars and polysaccharides (e.g., starch, dextrin, etc.), gums, water soluble polymers (e.g., PVOH), gelatin and other amino acid and/or protein-based materials, super absorbent materials, and porous media. Such a coating may assist with retention of the chemiluminescent component on the back sheet and/or reduce the tendency of the formulation to migrate or flow. The water soluble/permeable nature of the coating would not significantly impede transfer of the chemiluminescent component(s) by an aqueous system, such as during use of an absorbent article that includes the treated back sheet.

Other application/production methods in accordance with the present disclosure may be used to produce the treated materials described herein, such as the treated tissue composition, and indicator particles, and so forth.

For example, illustrative methods for producing the indicator particles, such as those shown in FIGS. 6A and 6B, may include a combination of techniques and other concepts described elsewhere herein, such as partitioning a fluff pulp sheet into particles having the desired shape and size, applying a suitable chemiluminescent component formulation to the particles (such as by spraying, soaking, and so forth), and removing the solvent and/or liquid carrier (such as by drying). The sequence of these operations may be varied; for example, a variation of such a method may include partitioning the fluff pulp sheet into particles after the application of the chemiluminescent component formulation and removal of the solvent and/or liquid carrier. In embodiments in which the particles are treated with both a luciferin and a luciferase, the formulation may include both components, or a variation of the method may include sequential treatment of the fluff pulp sheet and/or the particles with two different formulations of the respective components, and so forth. Such methods may be integrated into the production of an absorbent article, such as by mixing the indicator particles with an absorbent material (e.g., fluff pulp, synthetic fibers, SAP, and so forth). In some embodiments of this method, luciferin-treated particles may be mixed with luciferase-treated particles, or all of the indicator particles may be treated with the same chemiluminescent component, with the absorbent material treated with another chemiluminescent component, and so forth, such that both chemiluminescent components are integrated into the absorbent article.

A variant of a method of producing an absorbent article using the indicator particles includes delivering the indicator particles to a diaper structure as it is being produced, such as delivery of the particles to the surface of an absorbent core intended to face the back sheet, such that the indicator particles will be disposed in the diaper between the absorbent core and the back sheet; or delivery of the particles to the inner surface of the back sheet prior to final assembly of the diaper; or delivery within the absorbent core as performed for SAP; and so forth. Accordingly, in an embodiment, the method of producing an absorbent article includes producing an encapsulated material consisting of particles comprising one of two reactive components of a chemiluminescent system, which is configured to produce light upon contact with an aqueous system, with a material that is one or more of water permeable and water soluble; producing an absorbent article, including disposing the encapsulated material between a top sheet that is liquid permeable and a back sheet that is liquid-impermeable, and disposing the other reactive component of the chemiluminescent system in a structural element of the absorbent article.

As another example, in an illustrative method of producing a treated tissue composition, a formulation is applied to a surface of a liquid permeable tissue sheet, with the formulation including luciferin and a solvent in which the luciferin is dissolved. At least some of the solvent is then removed from the tissue sheet, with the luciferin being retained on the tissue sheet.

Somewhat similarly to the illustrative methods of producing a treated back sheet, embodiments of the illustrative method for producing a treated tissue composition include one or more various aspects and variations thereof, covering a broad range of application techniques. For example, tissue printing is one technique by which ink and similar formulations may be applied to a continuous tissue sheet, such as in a tissue coater (an example of such a machine is a MirWec μCoater™ 350), and a formulation suitable for application to the tissue sheet by delivery via a tissue coater is within the scope of this disclosure. To be suitable, such a formulation would be customized to the capabilities of the machine, which may include adjusting the viscosity of the formulation to an appropriate level, incorporating suitable additives, and so forth. Typically, the tissue coater applies the ink or formulation using one or more cylinders over which the tissue sheet is carried, followed by a heating and/or drying step in which the liquid carrier is removed. Typically, the tissue sheet to which the formulation is applied is a continuous tissue sheet.

As an alternative, in some embodiments, the formulation is applied by streaming the formulation to the tissue sheet surface, such as by means of a streaming apparatus, with the tissue sheet moving relative to the streaming apparatus. In some of such embodiments, the streaming apparatus includes one or more nozzles positioned to be proximate to and/or in contact with the moving surface. The tissue sheet, in such embodiments, may be a continuous tissue sheet.

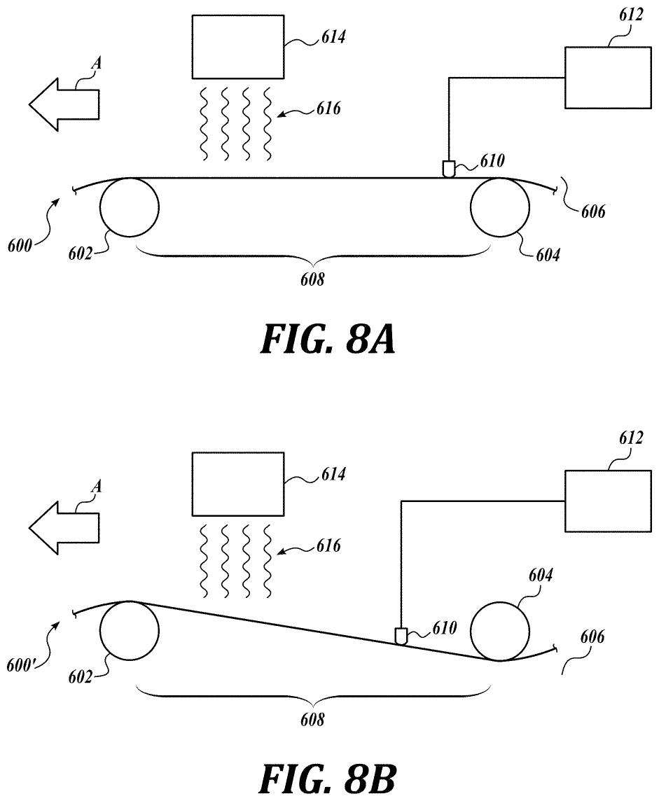

In accordance with one aspect of this disclosure, a schematic diagram of an example streaming apparatus suitable for use in such embodiments, is shown in FIGS. 8A and 8B. In FIG. 8A, streaming apparatus 600 is shown to include two pivot points in the form of rollers 602, 604 over which a continuous tissue sheet 606 is moved, in the direction indicated by arrow A. Tissue sheet 606 is thereby suspended, or floated, over a zone defined by the rollers, indicated in FIG. 8A as a tissue floating zone 608. The streaming apparatus 600 includes a nozzle 610 disposed to deliver a formulation to the top surface of the tissue sheet 606, such as by streaming the formulation, supplied from a reservoir 612, to the tissue sheet surface as the tissue sheet moves. Streaming apparatus 600 is also shown to include a heating element at 614, which provides a hot air zone 616 through which the treated tissue sheet is carried, to evaporate the solvent by heat treatment. FIG. 8B shows an illustrative alternative configuration of a streaming apparatus 600′, in which rollers 602, 604 have a different arrangement. A streaming apparatus having a configuration similar to that illustrated in FIGS. 8A and 8B was used to produce a treated tissue composition such as that shown as tissue composition 300 in FIG. 5A, such as with a longitudinal strip being discontinuous, dotted, dashed, straight, curved, according to one or more shapes, or the like. In some embodiments, the ambient temperature of the application environment provides sufficient thermal energy for drying of the liquid carrier of the formulation.

Several other variations to the configuration of a streaming apparatus are within the scope of this disclosure. For examples, a plurality of nozzles may be held stationary and selectively activated to produce a predetermined pattern or shape by activating and deactivating each nozzle at timed intervals. The rollers may be non-rotating cylinders. The formulation may be delivered via an arrangement of multiple nozzles. One or more nozzles may move relative to the tissue sheet, such as lateral to the direction in which the tissue sheet is moved, such as to produce a treated area that takes the form of a desired shape or pattern. To further this capability, one or more nozzles may be configured to stream the formulation intermittently and/or continuously, such as to produce a continuous or discontinuous treated area on the tissue sheet. Accordingly, various embodiments of the method include applying the formulation to the tissue sheet such that the treated area is any desired width, for example a longitudinal strip ranging from 0.1 mm to the width of the tissue sheet (for example, about 235 mm, the width of a standard diaper), in any desired arrangement or pattern (e.g., the treated area may be in the form of a strip that runs perpendicular or otherwise transverse to the longitudinal direction of the tissue sheet, continuous or discontinuous (such as dotted or dashed), straight or curved or including curved and/or straight portions, including shapes or other forms, and so forth). The treated area may range from 0.003-100% of the total area of the tissue sheet. As above, the total treated area may be anywhere from 1 to 99% of the area of the surface, for example from 1-2%, 1-10%, 5-20%, 1-25%, 10-50%, 20-90%, or within any range within any of these ranges, and all other possible subranges.

Embodiments of the method that incorporate such a streaming apparatus may offer some potential advantages over the use of a tissue printing machine. For example, a tissue floating zone may reduce the potential for bleed-through of the formulation through the tissue sheet to the cylinder(s) of a tissue printing machine. Also, floating the tissue may allow more contact with ambient air to dry the tissue, reducing the extent of heat treatment needed to remove solvent.

The formulation applied by embodiments of the method is not particularly limited, and several components and examples are discussed above, including sample compositions described as suitable for effective application of coelenterazine to a cellulosic tissue sheet. Accordingly, in some embodiments, the formulation includes 40 to 99 weight percent solvent and 0.01 to 20 weight percent luciferin. In some of such embodiments, the luciferin is coelenterazine. In some of such embodiments, the solvent is, or includes ethanol. In some of such embodiments, the solvent is, or includes ethanol and an excipient that facilitates the dissolution of the deposited luciferin on a treated tissue or a back sheet surface to chemiluminescent light producing reaction at an insult. In some of such embodiments, the solvent includes water and an excipient that facilitates the solubility of luciferin in water. In some embodiments, the formulation includes a binder and/or a viscosity adjusting agent, such as to adjust the viscosity to a desired level. In an embodiment, the formulation includes 0.01-30 weight percent binder. One of the aforementioned sample formulations included 87.39 weight percent ethanol, 0.88 weight percent ethyl cellulose (a binder), 0.33 weight percent polyurethane (also a binder), 0.40 weight percent corn starch and 11.0 weight percent of raw CTZ (at about 50% purity).

Embodiments of the method include applying the formulation to the tissue sheet at a rate to achieve a desired concentration of the chemiluminescent component(s) on the tissue sheet. As noted above, the concentration is not particularly limited, and may be expressed in various manners, such as a weight percent of the tissue sheet, a mass per unit length of the tissue sheet, and so forth. For example, in some embodiments in which the formulation includes luciferin, the formulation is applied at a rate to achieve a luciferin concentration on the tissue sheet from 0.00002 to 20 weight percent. In some embodiments in which the formulation includes coelenterazine, the formulation is applied at a rate to achieve a coelenterazine concentration on the tissue sheet from 0.01 mg-25 gram of coelenterazine per 12-inch length of tissue sheet. In some embodiments in which the formulation includes coelenterazine, the formulation is applied at a rate to achieve a coelenterazine concentration on the tissue sheet from 0.01-1.0 gram of coelenterazine per 12-inch length of tissue sheet. In some embodiments in which the formulation includes coelenterazine, the formulation is applied at a rate to achieve a coelenterazine concentration on the tissue sheet from 0.1-100 mg of coelenterazine per 12-inch length of tissue sheet.

Some embodiments of the method in accordance with the disclosure include additional steps, such as partitioning the treated tissue sheet into discrete lengths subsequent to application of the formulation. A related method of producing an absorbent core for incorporation into an absorbent article includes producing a treated tissue composition in accordance with the above, and incorporating the treated tissue composition into the absorbent core, such as by using the treated tissue composition as a wrapper for an absorbent material—that is, by partially or completely encompassing an absorbent material with the treated tissue composition. As discussed further herein, in an embodiment, such a treated tissue composition can include fibers, such as fibers selected from the group consisting of cellulose fibers, synthetic fibers, and combinations thereof. As noted above, application of the formulations disclosed herein, to a variety of substrate materials, may be done via any of a variety of methods, including streaming, printing, coating, spraying, rinsing, soaking, dipping, and so forth. The discussion above sets forth the principles of customizing a formulation for such applications, and example compositional ranges for illustrative applications. As noted above, one factor that may determine suitability for a particular application method is the viscosity of the formulation, which can be adjusted, for example, by means of incorporating one or more of the additive materials discussed herein (e.g., a binder, porous media, a releasing agent, other viscosity adjusting agents, and so forth). The viscosity ranges appropriate for certain applications may differ, or overlap.

The formulations in accordance with the present disclosure may be configured as inks suitable for various methods of ink application, such as streaming, printing, coating, and so forth. The following simplified descriptions illustrate differences among some application methods. Streaming, for example, refers to a process in which a flow of a formulation is applied to a surface by means of one or more nozzles (moving or stationary). Printing generally refers to processes in which an ink formulation is deposited onto a substrate material.

For example, in intaglio printing techniques, such as gravure and rotogravure, an image to be printed is engraved into an image carrier, which is then supplied with an ink formulation; the ink in the recessed area(s) forming the image is transferred to a substrate material pressed against the inked image carrier. In relief printing techniques, such as letterpress and flexography, the image to be printed is raised (instead of recessed) and supplied with ink, which is then transferred to a substrate material pressed against the relief plate. In screen-printing, a mesh is used to transfer ink onto a substrate, except in areas made impermeable to the ink, such as by a blocking stencil. Various coating methods, such as slot-die coating and ultra-strand coating, apply a formulation that usually includes a molten material onto a substrate material.

In inkjet printing techniques, droplets of ink are propelled against a substrate material. Most consumer and commercial/industrial inkjet printers use drop-on-demand (“DOD”) techniques, in which droplets are ejected from one or more ink chambers responsive to pulses of current. However, advancements in inkjet printing have produced several techniques that may allow for very rapid application of a variety of ink formulations to substrate materials and/or structural elements in accordance with the present disclosure, such as the ink formulations discussed herein. In some methods of continuous inkjet (“CU”) printing, for example, a printer imparts a controlled, variable electrostatic charge to individual ink droplets of a continuously produced stream. CIJ printing thus differs from DOD printing in that droplets are continuously ejected, which may afford greater speed in printing as compared to DOD techniques. The CIJ droplets are then propelled through a magnetic field generated in the printhead. A droplet is deflected toward the substrate material, with the extent of deflection determined by its electrostatic charge, with uncharged droplets being collected (e.g., inside the printhead) and cycled back into the ink supply, and in this manner characters or other images or patterns are printed on the substrate material.

As discussed above, illustrative methods of producing structural elements treated with one or more components of a chemiluminescent system, such as those shown in FIGS. 9A-9I, include applying a formulation to a predetermined area of a first surface of the structural element, wherein the formulation comprises the one or more components.

Accordingly, in an embodiment, the such methods include method of producing a structural element treated with at least one component of a chemiluminescent system, which reacts in the presence of an aqueous system to produce light, for incorporation into an absorbent article, the method comprising: applying a formulation to a predetermined area of a first surface of a structural element, wherein the formulation comprises the at least one component, and wherein the at least one component is selected from a luciferin and a luciferase.

In an embodiment, the predetermined area at least partially overlaps a region in which one or more fluid insults are expected during use of an absorbent article into which the structural element is incorporated, as discussed further herein with respect to FIGS. 9A-9J. Such a predetermined area can define a continuous shape. Correspondingly, the predetermined area can include two or more discrete portions.

The formulations applied to the structural element can include the formulations of the present disclosure, which include at least one component of a chemiluminescent system. In an embodiment, the at least one component is a luciferase. In an embodiment, the luciferase is selected from the group consisting of Gaussia luciferase, Renilla luciferase, Metridia luciferase, Oplophorus luciferase, dinoflagellate luciferase, copepod luciferase, and firefly luciferase. In an embodiment, the at least one component is a luciferin and a luciferase. In an embodiment, the luciferin is selected from the group consisting of coelenterazine, a coelenterazine analog, dinoflagellate luciferin, bacterial luciferin, fungal luciferin, firefly luciferin, vargulin, and furimazine, and/or wherein the luciferase is selected from the group consisting of Gaussia luciferase, Renilla luciferase, Metridia luciferase, Oplophorus luciferase, dinoflagellate luciferase, copepod luciferase, and firefly luciferase.

In some of such methods, the formulation is an ink formulation and the application includes printing, for example inkjet printing, such as CIJ printing. In some of such methods, both a luciferin and a luciferase are applied, for example in separate formulations, and/or to separate portions of the predetermined area, such as by CIJ or other inkjet printing. As described above, the other of the one or more components (i.e., the other of the luciferin or luciferase) may also be applied to the material of the structural element by a separate application method. The electrostatic charge of the ink formulations can be adjusted by the addition or organic or inorganic salts, including salts of the luciferin.

Further, versatile application techniques such as CIJ printing may be used for application of multiple formulations, such as ink formulations, to a substrate material. In an embodiment, applying a formulation includes applying at least two formulations, wherein one of the at least two formulations includes the luciferin, and wherein another of the at least two formulations includes the luciferase. Such application of at least two formulations can include applying a formulation includes separately applying the at least two formulations. In this regard, the at least two formulations can be applied, respectively, to non-overlapping portions of the predetermined area. As provided throughout, overlapping portions of the predetermined area may be treated with the two formulations in a manner that will minimize or avoid reacting the two components of the chemiluminescent system by more than 5% prior to the addition of an external source of water (i.e., a fluid insult).

As noted above, it may be desirable in some applications to apply other materials in addition to chemiluminescent materials to a substrate material or structural element, for example a non-chemiluminescent wetness indicator. Such variations are considered to be within the scope of this disclosure.

The methods of producing structural elements, in accordance with an embodiment of the disclosure can include, for example, applying a non-chemiluminescent wetness indicator to the first surface. Such a non-chemiluminescent wetness indicator can be applied to at least partially overlap the predetermined area. Likewise, the non-chemiluminescent wetness indicator is applied to not overlap the predetermined area.

As such, it is evident that these example application methods may have different requirements for a suitable formulation. However, the formulations in accordance with the present disclosure may be customized to a particular application method, such as by incorporating suitable additives (e.g., to achieve a desired viscosity, composition, thermal stability, rheological behavior, and so forth) while still effectively conveying the chemiluminescent component(s) of the formulation to the substrate material.

### EXAMPLES

The following paragraphs provide a description of illustrative manners in which treated materials having an incorporated chemiluminescent system are manufactured and tested. The results illustrate the benefits of the chemiluminescent system for detecting wetness in the dark. The chemiluminescence can be seen through the absorbent article into which it is incorporated, as well as through clothing. The examples are illustrative and not limiting.

While the chemiluminescent intensity in many of the examples is assessed by visibility to human eyes in low light or in a dark room, some of the examples discuss relative light unit (RLU) analysis performed by a luminometer, generally for comparative purposes. In such an RLU analysis, chemiluminescence is measured without any light filter. Roughly speaking, a value of 10,000,000 to 15,000,000 RLU corresponds to the lower threshold of visibility by human eyes in a dark room. However, higher or lower RLU levels may be required or permissible for visibility by some individuals.

### Porous Transfer Agent Example 1: Two-Component Formulation Preparation And Test

A solvent solution containing coelenterazine (“CTZ”) and Gaussia luciferase (GLuc) was prepared with a binder (ethyl cellulose (E-C 4 cP, from Sigma)) in ethanol. A sample formulation is shown in the table below.

TABLE 3
Example formulation for two-component (with binder) test

| Component | wt. % |
|---|---|
| Ethanol | 89.8 |
| E-C 4 cP | 8.6 |
| CTZ | <2.0 |
| GLuc | <2.0 |

20 μl of the above solvent solution was pipetted and drawn onto a poly back sheet as a roundish dot, and the poly sheet was dried at 80° C. for 30 seconds.

Once dried, the treated area was insulted with 0.2 ml of phosphate-buffered saline (“PBS”), pH 7.5. PBS is a water-based buffer solution that can be used to simulate urine in absorbent article testing. The formulation of the PBS used in the Examples herein includes 137 mM sodium chloride, 2.7 mM potassium chloride, 10 mM sodium phosphate dibasic, 1.8 mM potassium phosphate monobasic, 1.0 mM calcium chloride, and 0.5 mM magnesium chloride. Very low bioluminescence was observed, consistent with slow or reduced release of CTZ and/or GLuc from the poly sheet.

### Porous Transfer Agent Example 2: Two-Component Formulation with Porous Media Preparation and Test

A solvent solution containing CTZ and GLuc was prepared with various binders, for example ethyl cellulose (E-C 4 cP, from Sigma) and polyurethane (Versamid PUR 1120, from BASF), in ethanol. Porous media was also added, for example corn starch (Sigma Cat. No. 54180) and synthetic amorphous silica (SAS). Two sample formulations, identical except for the porous media used, are shown in the table below. Sample Formulation “A” contained corn starch. Sample Formulation “B” contained SAS.

TABLE 4
Example formulation for two-component
(with binder and porous media) test

| Component | Sample A | Sample B |
|---|---|---|
| Ethanol | 82.5 wt % | 82.5 wt % |
| E-C 4 cP | 8.8 wt % | 8.8 wt % |
| PUR | 3.3 wt % | 3.3 wt % |
| CTZ | <1.0 wt % | <1.0 wt % |
| GLuc | <2.0 wt % | <2.0 wt % |
| Corn starch | 4.0 wt % | — |
| SAS | — | 4.0 wt % |

The solvent solutions were each tested by pipetting 20 μl of the solution and drawing the solution down onto a poly back sheet as a roundish dot. The poly sheet was then dried at 80° C. for 30 seconds.

Once dried, each treated area was each insulted with 0.2 ml of pH 7.5 PBS. In both cases, extremely strong bioluminescence was observed, consistent with non-inhibited or assisted release of CTZ and/or GLuc from the poly sheet.

### Tissue Example 1: Preparation of Treated Tissue Sheet

Using a lab scale implementation of the floating tissue streaming design schematically shown at FIGS. 8A and 8B, formulations of coelenterazine (“CTZ”) were streamed onto a continuous, moving tissue sheet floated between two pivot points via a nozzle, to produce a treated area in the form of a longitudinal strip. Two sample formulations are shown in the table below. Sample Formulation “A” contained no binders. Sample Formulation “B” was prepared with binders ethyl cellulose (E-C 4 cP, from Sigma) and polyurethane (Versamid PUR 1120, from BASF), and corn starch.

TABLE 5
Example formulations for CTZ streaming onto tissue

| Component | Sample A | Sample B |
|---|---|---|
| Ethanol | 89.0 wt % | 87.39 wt % |
| Raw CTZ (~50% purity) | 11.0 wt % | 11.0 wt % |
| E-C 4 cP | — | 0.88 wt % |
| PUR | — | 0.33 wt % |
| Corn Starch | — | 0.40 wt % |

The formulation was streamed at a rate to apply 1.0 mg of CTZ (100% purity equivalent) per 12-inch length.

The streamed tissue was dried in an incubator at 80° C. for 15 sec to simulate a hot air zone. Higher temperatures can reduce drying time significantly.

The configuration schematically shown in FIGS. 8A and 8B was trialed successfully with similar formulations on a MirWec μCoater™ 350. Other tissue coating/printing apparatus would also be suitable.

### Tissue Example 2: Diaper Assembly with Treated Tissue Sheet and Insult Testing

A size 4 baby diaper was cut open and modified to include a diaper core that contained SAP and luciferase-treated fluff pulp. Streamed tissue samples prepared in Tissue Example 1 were placed between the back sheet and the modified diaper core. The diapers were reassembled and insulted with 45 ml of pH 7.5 PBS. After the insult was fully absorbed by the diaper, at T=0 hour, the chemiluminescence generated by the chemistry in the modified diapers was observed and imaged in a dark room without light. The chemiluminescence was visible after the insult. The modified diapers were subsequently insulted with the same dose of PBS buffer saline at T=2, 4, 6, and 8 hours. The chemiluminescence was visible after each insult.

### Tissue Example 3: Relative Light Unit (RLU) Analysis

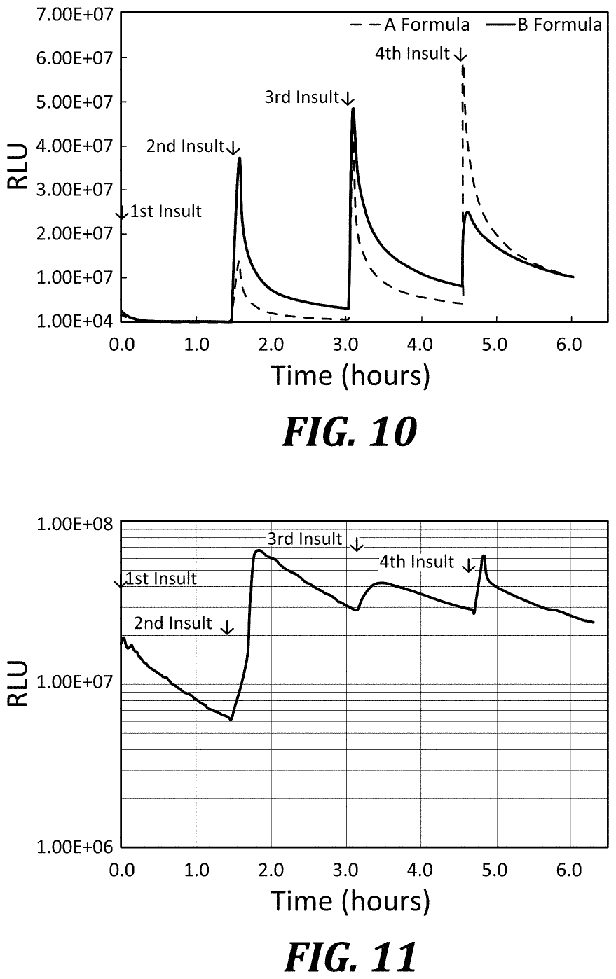

Reassembled diapers that were modified to include streamed tissue samples prepared with both sample formulations from Example 1 were cut with dies of 1-inch diameter to produce mini-diapers, which were insulted four consecutive times at 1.5 hour intervals with a 0.63 ml dose of pH 7.5 PBS, an amount equivalent to 45 ml of pH 7.5 PBS for the full diaper insult dose. The bioluminescence relative light units (RLU) were measured in a GloMax® Discover System Luminometer (Promega, Madison, Wis.). The RLU plot for the analysis, shown at FIG. 10, shows the level of chemiluminescence at and after each insult. The plots are based on the average of triplicate tests. The Sample A formulation samples exhibit lower light-production at 2^{nd} insult but much higher light-production at 4^{th} insult, while the Sample B formulation samples exhibit more light at 2^{nd} insult but less light at 4^{th} insult.

### Tissue Example 4: Staged Bioluminescence Glowing Design with Treated Tissue Sheet

Using a lab scale implementation of the floating tissue streaming design as done in Tissue Example 1, two formulations of coelenterazine (“CTZ”) were applied onto a continuous, moving tissue sheet floated between two pivot points via a nozzle, to produce a treated area in the form of two solid longitudinal strips. The two sample formulations contained binders and were identical except for the concentration of CTZ and the balance of solvent (Sample Formulation “C” had about 0.9% by weight CTZ at about 50% purity; Sample Formulation “D” had about twice the CTZ concentration of Formulation C). Sample Formulation C was applied to the tissue sheet in a line approximately three times the width as the Sample Formulation D line with same amount of volume of each being applied. The tissue sample was wetted to mimic an insult with a sufficient amount of a luciferase containing solution (about 2 mL with GLuc concentration of 1 mg/mL). At T=0, the “C” stream glowed brightly, while the “D” stream did not (not shown). At T=2 hr, the “C” stream continued to glow with minimal loss in intensity, while the “D” stream had only diffuse glowing at the periphery of the line (not shown). At T=4 hr, the “C” stream continued to glow with noticeable loss in intensity, while the “D” stream had increased intensity within the area of the line (not shown).

### Flake Particle Example 1: Preparation of Indicator Flakes

Pulp flakes with hexagon shapes are used for the preparation of pulp based indicator flakes. The hexagon pulp flakes have 1 mm thickness and four sides of 2 mm and two sides of 3 mm. Each flake is about 11 mg with a moisture content of about 7%. The flakes were prepared from 750 g/m² CF416 pulp board from International Paper.

A solvent solution containing coelenterazine (“CTZ”) and Gaussia luciferase (GLuc) was prepared with various binders, for example ethyl cellulose (E-C 4 cP, from Sigma) and polyurethane (Versamid PUR 1120, from BASF). The GLuc was finely ground prior to adding to the solution, to facilitate dispersion. A sample formulation is shown in the table below.

TABLE 6
Example formulation for treatment of pulp based indicator flakes

| Component | wt. % |
|---|---|
| Ethanol | 98.449 |
| E-C 4 cP | 0.535 |
| PUR | 0.203 |
| CTZ | <1.0 |
| GLuc | <1.0 |
| Corn starch | 0.242 |

The solution was yellow in color. Once well mixed, the solution was added to each flake. A total of 0.7 g of pulp hexagon flakes (about 65 flakes) were treated and used for each diaper application. After treatment, the pulp flakes were dried with hot air in an oven at 80° C. for 2 minutes.

In this example, the dried flakes included CTZ in a concentration less than 0.50 weight % and GLuc in a concentration less than 0.01 weight %. 0.7 g of dried flakes included a total of 1.0 mg of CTZ and 0.025 mg of GLuc.

### Flake Particle Example 2: Diaper Assembly with Indicator Flakes

A longitudinal, rectangular section of the back sheet of a size 4 baby diaper was cut open and peeled back without interrupting the diaper core structure. The flakes were evenly distributed in a single, non-contiguous layer on the inner surface of the back sheet section. The section of back sheet was then sealed back to the diaper with transparent tape placed over the cut edges.

### Flake Particle Example 3: Repeated Diaper Insults with PBS

The diaper in Example 2 was insulted with 60 ml of pH 7.5 PBS.

After the insult was fully absorbed by the diaper, at T=0 hour, the chemiluminescence generated by the chemistry on the treated pulp flakes were observed and imaged in a dark room without light. The chemiluminescence was extremely visible after the insult. By T=1.5 hours, the chemiluminescence was no longer visible. A second insult was applied at T=1.5 hours, and the chemiluminescence was again very visible. By T=2.0 hours, the chemiluminescence was no longer visible. A third insult was applied at T=3.0 hours, and the chemiluminescence was again visible, although less than after the first and second insults. By T=4.0 hours, the chemiluminescence was no longer visible.

### Flake Particle Example 4: Relative Light Unit (RLU) Analysis

A one-inch diameter disc was punched out of a diaper. The back sheet was removed, and 5 treated hexagon pulp flakes treated according to Example 1 were placed in a row between the back sheet and the diaper core. Four consecutive insults of pH 7.5 PBS were performed at 1.5-hour intervals. The chemiluminescence was monitored by a GloMax® Discover System Luminometer (Promega, Madison, Wis.). The RLU plot for the analysis, presented as FIG. 11, shows the level of chemiluminescence at and after each insult. The results show that the glowing intensity (RLU) spikes after the first insult, then gradually decreases. At second insult, the spike is much higher, then decreases and maintains an intensity above the first insult spike until gradually decreasing in intensity after the 4^{th} insult.

### Strip Particle Example 1: Preparation of Indicator Strips

Pulp strips of 1 mm×2 mm×350 mm are cut and used for the preparation of pulp based indicator strips. Each strip weighs about 0.7 g. The strips were prepared using 750 g/m² CF416 pulp board from International Paper.

A solvent solution containing coelenterazine (“CTZ”) and suspended Gaussia luciferase (GLuc) was prepared with various binders, for example ethyl cellulose (E-C 4 cP, from Sigma) and polyurethane (Versamid PUR 1120, from BASF). The GLuc was finely ground prior to adding to the solution, to facilitate dispersion. A sample formulation is shown in the table below.

TABLE 7
Example formulation for treatment of pulp based
indicator particles

| Component | wt. % |
|---|---|
| Ethanol | 98.449 |
| E-C 4 cP | 0.535 |
| PUR | 0.203 |
| CTZ | <1.0 |
| GLuc | <0.50 |
| Corn starch | 0.242 |

The solution was yellow in color. Once well mixed, the solution was added to each strip. After treatment, the pulp strips were dried with hot air in an oven at 80° C. for 2 minutes.

In this example, the dried strips each included CTZ in a concentration less than 0.50 weight % and GLuc in a concentration less than 0.10 weight %. Each dried strip included a total of 1.0 mg of CTZ and 0.025 mg of GLuc.

### Strip Particle Example 2: Diaper Assembly with Indicator Strips

A longitudinal, rectangular section of the back sheet of a size 4 baby diaper was cut open and peeled back without interrupting the diaper core structure. A single strip was placed longitudinally in the center of the inner surface of the back sheet section. The section of back sheet was then sealed back to the diaper with transparent tape placed over the cut edges.

### Strip Particle Example 3: Repeated Diaper Insults With PBS

The diaper in Example 2 was insulted with 60 ml of pH 7.5 PBS.

After the insult was fully absorbed by the diaper, at T=0 hour, the chemiluminescence generated by the chemistry on the treated pulp flakes were observed and imaged in a dark room without light. The chemiluminescence was extremely visible after the insult. By T=1.5 hours, the chemiluminescence was faintly visible. A second insult was applied at T=1.5 hours, and the chemiluminescence was again very visible. By T=2.0 hours, the chemiluminescence was faintly visible. A third insult was applied at T=3.0 hours, and the chemiluminescence was again visible, although notably less than after the second insult. By T=4.0 hours, the chemiluminescence was no longer visible.

### Strip Particle Example 4: Relative Light Unit (RLU) Analysis

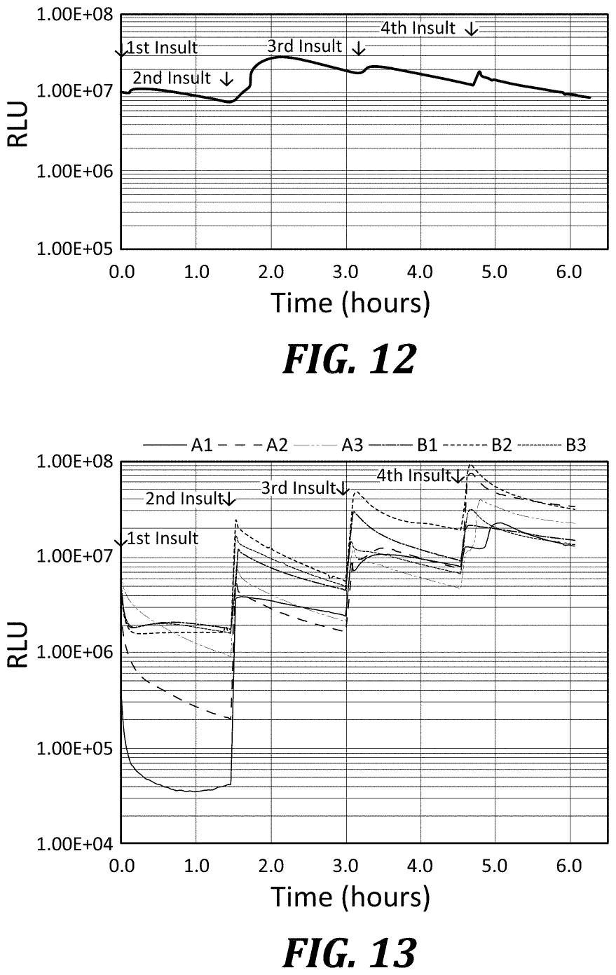

A one-inch diameter disc was punched out of a diaper. The back sheet was removed, and a 1-inch treated pulp strip treated according to Example 1 was placed between the back sheet and the diaper core. Four consecutive insults of pH 7.5 PBS were performed at 1.5 hours intervals. The chemiluminescence was monitored by a GloMax® Discover System Luminometer (Promega, Madison, Wis.). The RLU plot, presented as FIG. 12, shows the level of chemiluminescence at and after each insult. The results show that the glowing intensity (RLU) increases after the first insult, then gradually decreases. At second insult, the spike is much higher, then decreases and maintains an intensity above the first insult spike until gradually decreasing in intensity after the 4^{th} insult. From T=2.0 hours (following the spike after the second insult) to T=4.0 hours, the chemiluminescence was fairly stable and visible to human eyes in a dark room.

### Strip Particle Example 5: Production of CTZ-Treated Indicator Strips of Various Dimensions

CF416 pulp board (basis weight 750 g/m², International Paper) was processed in a Crumbler® rotary shear system (Forest Concepts, LLC) with a 1.8 mm blade cutter and a 0.8 mm blade cutter to produce strips of pulp having the respective widths. The strips were sorted by length as long strips (e.g., longer than 20 mm) and short strips (10-20 mm). The apparent density or bulk of the 1.8 mm strips was 0.0886 cc/g and that of the 0.8 mm strips was 0.145 cc/g. Strips of various dimensions were used in order to test aspects of the production process, such as speed and efficiency, as well as to determine whether smaller strips could be produced as efficiently. Smaller strips may be more pneumatic-transportable, and thus more suitable for use in standard diaper fluff core forming apparatus.

A solvent solution containing coelenterazine (“CTZ”) was prepared with various binders, for example ethyl cellulose (E-C 4 cP, from Sigma) and polyurethane (Versamid PUR 1120, from BASF). Corn starch was also added (Sigma Catalog No. S4180). A sample formulation is shown in the table below. One gram of the sample composition contains 2.0 mg CTZ.

TABLE 8
Example formulation for treatment of pulp based indicator strips

| Component | wt. % |
|---|---|
| Ethanol | 98.19 |
| E-C 4 cP | 0.88 |
| PUR | 0.33 |
| CTZ | <0.50 |
| Corn starch | <0.50 |

Once well mixed, the solution was applied to the strips by mixing 2.0 g portions of the solution with 2.0 g of the various pulp strips in a small beaker. After treatment, the pulp strips were dried with hot air in an oven at 80° C. for 2 minutes.

### Strip Particle Example 6: Diaper Core Assembly with CTZ-Treated Indicator

Strips and Insult Testing One gram of CTZ-treated strips of the aforementioned sizes (each gram containing a total of 2.0 mg CTZ) were sprinkled onto the back surface of a diaper core that contained luciferase-treated fluff pulp (at a concentration of approximately 0.56 mg luciferase per gram of fluff, or approximately 5.6 mg of luciferase per diaper core). The diaper core assembly was then sandwiched between a liquid-impermeable poly back sheet and a liquid permeable top sheet, with the CTZ-treated strips disposed adjacent the back sheet.

An insult of 60 ml of pH 7.5 PBS was added to the diaper core. The chemiluminescence was observed and imaged, and was quite visible through the back sheet in a dark room, and also under dim light.

### Back Sheet Example 1: CTZ Formulation for Back Sheet Application

A solvent solution containing coelenterazine (“CTZ”) was prepared with various binders, for example ethyl cellulose (E-C 4 cP, from Sigma) and polyurethane (Versamid PUR 1120, from BASF). The binders were diluted in ethanol, and then CTZ was added. Once dissolved, corn starch was added. The mixture was mixed by vortexing. A sample formulation is shown in the table below.

TABLE 9
Example formulation for treatment of back sheet

| Component | wt. % |
|---|---|
| Ethanol | 47.10 |
| E-C 4 cP | 1.49 |
| PUR | <1.0 |
| CTZ | <0.50 |
| Corn starch | 50.67 |

Corn starch did not exhibit agglomeration in ethanol and similar formulations up to 52 weight percent starch were found to remain flowable.

### Back Sheet Example 2: Application and Wicking-Facilitated Drying

A small syringe (1 ml) was used to stream the formulation from Example 1 in a six-inch line across a poly back sheet (XP-1943SX poly film, Berry Global Inc., Evansville, Ind.), so that 64 mg of the formulation per inch of the back sheet (about 0.05 to 0.30 mg CTZ per inch) was applied.

Immediately after application, a diaper core made of fluff pulp treated with luciferase in a concentration less than 1.0 weight percent, and SAP, was laid down over the streamed line on the back sheet.

The fluff core wicked the solvent (ethanol, in this case) more quickly than air-drying.

### Back Sheet Example 3: Chemiluminescence Testing

Samples of (1) the assembly of treated back sheet and diaper core, and (2) the diaper core only, were dosed with pH 7.5 PBS. The samples of the assembly of the treated back sheet and diaper core exhibited strong bioluminescence. The samples of the diaper core only also exhibited some bioluminescence, indicating that some of the CTZ had diffused into the diaper core when used to wick the solvent.

### Back Sheet Example 4: Relative Light Unit (RLU) Analysis

A standard diaper top sheet with acquisition distribution layer (ADL) was applied to the bare side of the diaper core of the assembly from Example 3, to sandwich the diaper core between the top sheet/ADL and the treated back sheet. Six one-inch diameter discs were punched out of this assembly to produce six “mini-diaper” assemblies. These were placed in a six-sample test cassette designed for bioluminescence testing in a GloMax® Discover System Luminometer (Promega, Madison, Wis.). Each mini-diaper was insulted with a 0.84 ml dose of pH 7.5 PBS buffer solution, an amount proportional to a 60 ml insult delivered to a medium-size diaper. Consecutive insults were performed at 1.5-hour intervals. The RLU plot for the analysis, presented as FIG. 13, shows the level of chemiluminescence at and after each insult. The results show that the glowing intensity (RLU) increases after the first insult (at T=0), then gradually decreases. There is a spike after each successive insult, then a successively slower decrease. The results show that in this application, the more liquid present correlates to more intense light.

In addition, the chemiluminescence after the 4^{th} insult was observed to be visible in a dark room, despite the comparatively small treated area (a one-inch line, in each mini-diaper) relative to a fully-treated baby diaper core.

### SAP Level Example: Varying Amounts of Super Absorbent Polymer Affects Water Availability in the Aqueous System

Diaper cores were made with different contents of superabsorbent polymer (SAP): 0, 6, 12, 18, 24 and 36 weight percentage based on total cellulose fibers (fluff) in the core. The cellulose fibers were treated with luciferase (GLuc). Tissue were streamed to have a concentration of pure coelenterazine (“CTZ”) less than 1.0 mg, with a formulation of CTZ (about 50% purity) in ethanol with ethyl cellulose (E-C 4 cP) 0.88 wt %, polyurethane (PUR) 0.33 wt %, and corn starch 0.40 wt %.

The diaper cores with luciferase (GLuc) containing cellulose fibers and different SAP contents were modified to include the tissue treated with the CTZ formulation. In each case, the treated tissue was between the diaper core and the diaper back sheet. The modified diapers were cut with dies of 1-inch diameter to produce mini-diapers, which were insulted four consecutive times at 2 hour intervals with a 0.63 ml dose of pH 7.5 PBS, an amount equivalent to 45 ml of pH 7.5 PBS for the full diaper insult dose. The bioluminescence relative light units (RLU) were measured in a GloMax® Discover System Luminometer (Promega, Madison, Wis.). The RLU plots are showed in FIG. 14A for the SAP contents of 0, 6 and 12 weight percentage, and in FIG. 14B for the SAP contents of 18, 24 and 36 weight percentage.

Mineral Salt Application Example

PBS buffer was used as the carrier solution for treating pulp strips with aluminum chloride. Each strip of pulp was treated with its weight of aluminum chloride containing PBS buffer. For example, if it was desired to have a pulp that was treated with 1% aluminum chloride a PBS buffer containing 1% aluminum chloride was used. If it was desired to have a pulp that was treated with 2% aluminum chloride a PBS buffer containing 2% aluminum chloride was used, and so forth.

After treatment the pulp strips were allowed to dry and were then fiberized in a Kamas hammermill.

Pads were formed by mixing 7.4 g fiber with 4.2 g of BASF T9400 SAP in a 6″ pad former. The pads were then pressed at 50 psi for 20 seconds.

Mineral salt addition of aluminum and magnesium was found to inhibit reaction conditions after early insults due to decreased (unfavorable) pH. More insults result in a buffered or increased pH, which then favors reaction conditions, resulting in higher light production peaks after several insults (see FIG. 16).

The addition of other mineral salts that do not decrease pH will counteract the effect of SAP by increasing osmotic pressure in the fluff pulp, for example. Thus, more free water will be available for the aqueous system to start the reaction of the chemiluminescent system.

### CONCLUSION AND ILLUSTRATIVE EMBODIMENTS

The various illustrative ranges provided for the components and materials discussed herein are each intended to encompass the example upper and/or lower bounds of the given range, as well as any and all sub-ranges within the given range, without explicitly reciting such sub-ranges. Likewise, the various illustrative combinations of the components, steps, processes, materials, concepts, and principles discussed above, for example in the various treated materials, absorbent articles, formulations, treatment methods, production methods, and so forth, are intended to encompass all combinations thereof that would be evident to an artisan given this disclosure, without explicitly reciting such combinations. As such, while illustrative embodiments have been illustrated and described, it will be appreciated that various changes can be made therein without departing from the spirit and scope of the disclosure.

As used herein, the word “about” as it relates to a quantity indicates a number within range of minor variation above or below the stated reference number. For example, “about” can refer to a number within a range of 10%, 9%, 8%, 7%, 6%, 5%, 4%, 3%, 2%, or 1% above or below the indicated reference number. In some embodiments, “about” refers to a number within a range of 5% above or below the indicated reference number. In some embodiments, “about” refers to a number within a range of 10% above or below the indicated reference number. In some embodiments, “about” refers to a number within a range of 1% above or below the indicated reference number.

Illustrative, non-exclusive examples of inventive subject matter according to the present disclosure are described in the following enumerated paragraphs:

1. A treated tissue composition comprising:

a liquid permeable tissue sheet comprising cellulosic fibers and having two opposed surfaces;

wherein at least one surface is treated with at least one component of a chemiluminescent system, wherein the chemiluminescent system is adapted to react in the presence of an aqueous system to produce light;

wherein the at least one component is selected from a luciferin and a luciferase; and

wherein the at least one component is retained on the at least one surface.

1.1. The treated tissue composition of paragraph 1, wherein the at least one component is a luciferin.

1.1.1. The treated tissue composition of paragraph 1.1, wherein the luciferin is selected from the group consisting of coelenterazine, a coelenterazine analog, dinoflagellate luciferin, bacterial luciferin, fungal luciferin, firefly luciferin, vargulin, and furimazine.

1.1.2. The treated tissue composition of paragraph 1.1, wherein the luciferin is coelenterazine.

1.1.2.1. The treated tissue composition of paragraph 1.1.1, comprising 0.00002 to 20 weight percent coelenterazine.

1.1.2.1.1. The treated tissue composition of paragraph 1.1.2.1, comprising 0.00002 to 0.01 weight percent coelenterazine.

1.1.2.1.2. The treated tissue composition of paragraph 1.1.2.1, comprising 0.01 to 2 weight percent coelenterazine.

1.1.2.1.3. The treated tissue composition of paragraph 1.1.2.1, comprising 2 to 10 weight percent coelenterazine.

1.1.2.1.3.1. The treated tissue composition of paragraph 1.1.2.1.3, comprising 1 to 6 weight percent coelenterazine.

1.1.2.1.4. The treated tissue composition of paragraph 1.1.2.1, comprising 10 to 20 weight percent coelenterazine.

1.1.2.2. The treated tissue composition of paragraph 1.1.1, wherein the tissue sheet is 0.1-235 mm wide, and comprises 0.01 mg-25 g of coelenterazine per 12-inch length, with the weight of the coelenterazine per 12-inch length being less than or equal to the weight of a 12-inch length of untreated tissue sheet.

1.1.2.3. The treated tissue composition of paragraph 1.1.1, wherein the tissue sheet is 0.1-235 mm wide, and comprises 0.1-100 mg of coelenterazine per 12-inch length, with the weight of the coelenterazine per 12-inch length being less than or equal to the weight of a 12-inch length of untreated tissue sheet.

1.2. The treated tissue composition of any of paragraphs 1-1.1.1.2, wherein the at least one component is retained on the at least one surface by a binder.

1.2.1. The treated tissue composition of paragraph 1.2, wherein the binder comprises one or more binders selected from the group consisting of ethyl cellulose, methyl cellulose, nitrocellulose, and polyurethane.

1.3. The treated tissue composition of any of paragraphs 1-1.2, wherein the tissue sheet has a basis weight of 10-1500 g/m².

1.4. The treated tissue composition of any of paragraphs 1-1.3, wherein the tissue sheet further comprises synthetic fibers.

1.5. The treated tissue composition of any of paragraphs 1-1.4, wherein the area of the at least one surface treated with the component has a width less than or equal to the width of the tissue sheet.

1.5.1. The treated tissue composition of paragraph 1.5, wherein the treated area is in the form of a longitudinal strip.

1.5.1.1. The treated tissue composition of paragraph 1.5.1, wherein the longitudinal strip is continuous.

1.5.1.2. The treated tissue composition of paragraph 1.5.1, wherein the longitudinal strip is discontinuous.

1.5.1.2.1. The treated tissue composition of paragraph 1.5.1.2, wherein the longitudinal strip is one or more of dotted and dashed.

1.5.1.3. The treated tissue composition of any of paragraphs 1.5.1-1.5.1.2.1, wherein the longitudinal strip is straight.

1.5.1.4. The treated tissue composition of any of paragraphs 1.5.1-1.5.1.2.1, wherein the longitudinal strip includes one or more curved portions.

1.5.1.5. The treated tissue composition of any of paragraphs 1.5.1-1.5.1.2.1, wherein the longitudinal strip includes one or more straight portions.

1.5.2. The treated tissue composition of paragraph 1.5, wherein the treated area is in the form of one or more shapes.

1.5.3. The treated tissue composition of paragraph 1.5, wherein the total treated area is 0.003-100% of the area of the tissue sheet.

1.6. The treated tissue composition of paragraph 1, wherein the at least one component is a luciferase.

1.6.1. The treated tissue composition of paragraph 1.6, wherein the luciferase is selected from the group consisting of Gaussia luciferase, Renilla luciferase, Metridia luciferase, Oplophorus luciferase, dinoflagellate luciferase, copepod luciferase, and firefly luciferase.

1.7. The treated tissue composition of any of paragraphs 1-1.6, wherein the at least one component is a luciferin and a luciferase.

1.7.1. The treated tissue composition of paragraph 1.7, wherein the luciferin is selected from the group consisting of coelenterazine, a coelenterazine analog, dinoflagellate luciferin, bacterial luciferin, fungal luciferin, firefly luciferin, vargulin, and furimazine, and/or wherein the luciferase is selected from the group consisting of Gaussia luciferase, Renilla luciferase, Metridia luciferase, Oplophorus luciferase, dinoflagellate luciferase, copepod luciferase, and firefly luciferase.

1.7.2. The treated tissue composition of paragraph 1.7 or 1.7.1, wherein the luciferin is coelenterazine and the luciferase is one or more of Gaussia luciferase, Renilla luciferase, and Metridia luciferase.

1.8. The treated tissue composition of any of paragraphs 1-1.7.2, further comprising a releasing agent adapted to facilitate the release of the at least one component from the at least one surface in the presence of an aqueous system.

1.9. The treated tissue composition of any of paragraphs 1-1.8, comprising at least two liquid permeable layers, one of which is the tissue sheet.

1.9.1. The treated tissue composition of paragraph 1.9, comprising a first and a second liquid permeable tissue sheet, wherein at least one surface of each tissue sheet is treated with a different component of the chemiluminescent system.

1.9.2. The treated tissue composition of paragraph 1.9 or 1.9.1, wherein the tissue sheet is sandwiched between two liquid permeable layers.

1.10. An absorbent core for an absorbent article comprising the treated tissue composition of any of paragraphs 1-1.9.2.

1.10.1. The absorbent core of paragraph 1.10, comprising an absorbent structure that is at least partially encompassed by the treated tissue composition of any of paragraphs 1-1.9.2.

1.10.2. An absorbent article comprising the absorbent core of paragraph 1.10 or 1.10.1.

1.11. An absorbent core for an absorbent article, comprising an absorbent structure that is at least partially encompassed by the treated tissue composition of any of paragraphs 1-1.1.1.2, wherein the treated tissue composition comprises luciferin, and wherein the absorbent structure comprises luciferase.

2. An indicator particle comprising hydrogen bonded cellulose pulp fibers and at least one component of a chemiluminescent system, wherein the chemiluminescent system is adapted to react in the presence of an aqueous system to produce light;

wherein the at least one component is selected from a luciferin and a luciferase; and

wherein the at least one component is retained on the cellulose pulp fibers.

2.1. The indicator particle of paragraph 2, wherein the at least one component is disposed on the cellulose fibers of at least one surface of the particle.

2.2. The indicator particle of paragraph 2, wherein the at least one component is disposed on the cellulose pulp fibers throughout the particle.

2.3. The indicator particle of paragraph 2, wherein the at least one component is a luciferin.

2.3.1. The indicator particle of paragraph 2.3, wherein the luciferin is selected from the group consisting of coelenterazine, a coelenterazine analog, dinoflagellate luciferin, bacterial luciferin, fungal luciferin, firefly luciferin, vargulin, and furimazine.

2.4. The indicator particle of paragraph 2, wherein the at least one component is a luciferase.

2.4.1. The indicator particle of paragraph 2.4, wherein the luciferase is selected from the group consisting of Gaussia luciferase, Renilla luciferase, Metridia luciferase, Oplophorus luciferase, dinoflagellate luciferase, copepod luciferase, and firefly luciferase.

2.4.2. The indicator particle of paragraph 2.4, wherein the luciferase is one of more of Gaussia luciferase, Renilla luciferase, and Metridia luciferase.

2.5. The indicator particle of paragraph 2, comprising both a luciferin and a luciferase,

wherein the luciferin is selected from the group consisting of coelenterazine, a coelenterazine analog, dinoflagellate luciferin, bacterial luciferin, fungal luciferin, firefly luciferin, vargulin, and furimazine; and

wherein the luciferase is selected from the group consisting of Gaussia luciferase, Renilla luciferase, Metridia luciferase, Oplophorus luciferase, dinoflagellate luciferase, copepod luciferase, and firefly luciferase.

2.5.1. The indicator particle of paragraph 2.5, wherein the luciferin is coelenterazine and wherein the luciferase is one or more of Gaussia luciferase, Renilla luciferase, and Metridia luciferase.

2.5.1.1. The indicator particle of paragraph 2.5.1, wherein the particle comprises 0.00002-20.0 weight percent coelenterazine.

2.5.1.1.1. The indicator particle of paragraph 2.5.1.1, wherein the particle comprises 0.00002-0.01 weight percent coelenterazine.

2.5.1.1.2. The indicator particle of paragraph 2.5.1.1, wherein the particle comprises 0.01-0.20 weight percent coelenterazine.

2.5.1.1.2. The indicator particle of paragraph 2.5.1.1, wherein the particle comprises 0.2-5.0 weight percent coelenterazine.

2.5.1.1.3. The indicator particle of paragraph 2.5.1.1, wherein the particle comprises 5.0-20.0 weight percent coelenterazine.

2.5.1.2. The indicator particle of any of paragraphs 2.5.1-2.5.1.1.3, wherein the particle comprises 0.0003-10.0 weight percent luciferase.

2.5.1.2.1. The indicator particle of paragraph 2.5.1.2, wherein the particle comprises 0.001-0.10 weight percent luciferase.

2.5.1.2.2. The indicator particle of paragraph 2.5.1.2, wherein the particle comprises 0.001-0.10 weight percent luciferase.

2.5.1.2.3. The indicator particle of paragraph 2.5.1.2, wherein the particle comprises 0.10-2.0 weight percent luciferase.

2.5.1.2.4. The indicator particle of paragraph 2.5.1.2, wherein the particle comprises 2.0-10 weight percent luciferase.

2.5.1.3. The indicator particle of paragraph 2.5.1, wherein the particle comprises 0.00002-20.0 weight percent coelenterazine and 0.0003-10.0 weight percent Gaussia luciferase, Renilla luciferase, and/or Metridia luciferase.

2.6. The indicator particle of any of paragraphs 2-2.5.1.3, having a length, a width, and a thickness;

wherein the length is greater than or equal to the width; and

wherein the width is greater than or equal to the thickness.

2.6.1. The indicator particle of paragraph 2.6, wherein the ratio of the length to the width is less than 1.5.

2.6.1.1. The indicator particle of paragraph 2.6.1, wherein the product of the length and the width is between 0.1-300 mm².

2.6.1.2. The indicator particle of paragraph 2.6.1, wherein the product of the length and the width is between 8-30 mm².

2.6.2. The indicator particle of paragraph 2.6, wherein the ratio of the length to the width is greater than or equal to 1.5.

2.6.2.1. The indicator particle of paragraph 2.6.2, wherein the cross-sectional area of the indicator particle is 0.01-200 mm².

2.6.2.2. The indicator particle of paragraph 2.6.2 or 2.6.2.1, having a length of 1-800 mm.

2.6.2.3. The indicator particle of paragraph 2.6.2 or 2.6.2.1, having a length of 1-350 mm.

2.6.2.4. The indicator particle of any of paragraphs 2.6.2-2.6.2.3, having a width of 0.5-2.5 mm and a thickness of 0.05-2.0 mm.

2.7. The indicator particle of any of paragraphs 2-2.6.2.4, further comprising synthetic fibers.

2.7.1. The indicator particle of paragraph 2.7, wherein the fiber diameter of the synthetic fibers is 1-100 microns.

2.8. The indicator particle of any of paragraphs 2-2.7.1, having a basis weight of 10-850 g/m².

2.9. The indicator particle of any of paragraphs 2-2.8, further comprising a binder, wherein the binder retains the at least one component on the cellulose pulp fibers.

2.9.1. The indicator particle of paragraph 2.9, wherein the binder comprises one or more binders selected from the group consisting of ethyl cellulose, methyl cellulose, nitrocellulose, and polyurethane.

2.9.1.1. The indicator particle of paragraph 2.9.1, wherein the at least one component is luciferin.

2.10. The indicator particle of any of paragraphs 2-2.9.1.1, further comprising a porous transfer agent adapted, upon contact of the indicator particle with an aqueous system, to facilitate transfer of the at least one component, and/or the aqueous system, relative to the indicator particle.

2.10.1. The indicator particle of paragraph 2.10, wherein the porous transfer agent is selected from the group consisting of starch, amorphous silica, clay minerals, cellulose pulp fiber, cotton fiber, and synthetic polymer fiber.

2.11. The indicator particle of any of paragraphs 2-2.10.1, further comprising a releasing agent adapted to facilitate the release of the at least one component from the cellulose pulp fibers in the presence of an aqueous system.

2.12. An absorbent article comprising a plurality of the indicator particle of any of paragraphs 2-2.11.

2.12.1. The absorbent article of paragraph 2.12, further comprising a top sheet that is liquid permeable, a back sheet that is liquid impermeable, and absorbent material disposed between the top sheet and the back sheet;

wherein the indicator particles are disposed between the top sheet and the back sheet.

2.12.2. The absorbent article of paragraph 2.12 or 2.12.1, wherein the indicator particles are disposed between the absorbent material and the back sheet.

2.12.3. The absorbent article of any of paragraphs 2.12-2.12.2, wherein each of the plurality of indicator particles comprises both a luciferin and a luciferase.

2.12.3.1. The absorbent article of paragraph 2.12.3,

wherein the luciferin is selected from the group consisting of coelenterazine, a coelenterazine analog, dinoflagellate luciferin, bacterial luciferin, fungal luciferin, firefly luciferin, vargulin, and furimazine; and

wherein the luciferase is selected from the group consisting of Gaussia luciferase, Renilla luciferase, Metridia luciferase, Oplophorus luciferase, dinoflagellate luciferase, copepod luciferase, and firefly luciferase.

2.12.3.1.1. The absorbent article of paragraph 2.12.3.1, wherein the luciferin is coelenterazine and the luciferase is one or more of Gaussia luciferase, Renilla luciferase, and Metridia luciferase; and

wherein the plurality of indicator particles collectively contain 0.0001-20.0 mg of coelenterazine and 0.00003-20.0 mg total of luciferase.

3. An article, comprising:
synthetic fibers; and

at least one component of a chemiluminescent system that is adapted to react in the presence of an aqueous system to produce light;

wherein the at least one component is selected from a luciferin and a luciferase.

3.1. The article of paragraph 3, wherein the synthetic fibers form an absorbent matrix.

3.2. The article of paragraph 3.1, wherein the absorbent matrix comprises of synthetic fibers.

3.3. The article of any of paragraphs 3-3.2, wherein the at least one component is a luciferin.

3.3.1. The article of paragraph 3.3, wherein the luciferin is selected from the group consisting of coelenterazine, a coelenterazine analog, dinoflagellate luciferin, bacterial luciferin, fungal luciferin, firefly luciferin, vargulin, and furimazine.

3.4. The article of any of paragraphs 3-3.2, wherein the at least one component is a luciferase.

3.4.1. The article of paragraph 3.4, wherein the luciferase is selected from the group consisting of Gaussia luciferase, Renilla luciferase, Metridia luciferase, Oplophorus luciferase, dinoflagellate luciferase, copepod luciferase, and firefly luciferase.

3.4.1.1. The article of paragraph 3.4.1, wherein the luciferase is Gaussia luciferase.

3.4.1.2. The article of paragraph 3.4.1, wherein the luciferase is Renilla luciferase.

3.4.1.3. The article of paragraph 3.4.1, wherein the luciferase is Metridia luciferase.

3.4.1.4. The article of paragraph 3.4.1, wherein the luciferase is two or more of Gaussia luciferase, Renilla luciferase, and Metridia luciferase.

3.5. The article of any of paragraphs 3-3.2, comprising both a luciferin and a luciferase,

wherein the luciferin is selected from the group consisting of coelenterazine, a coelenterazine analog, dinoflagellate luciferin, bacterial luciferin, fungal luciferin, firefly luciferin, vargulin, and furimazine; and

wherein the luciferase is selected from the group consisting of Gaussia luciferase, Renilla luciferase, Metridia luciferase, Oplophorus luciferase, dinoflagellate luciferase, copepod luciferase, and firefly luciferase.

3.5.1. The article of paragraph 3.5.1, wherein the luciferase is one or more of Gaussia luciferase, Renilla luciferase, and Metridia luciferase.

3.6. The article of any of paragraphs 3-3.5.1, wherein the article is configured as an absorbent core for use in an absorbent article.

3.6.1. The article of paragraph 3.6, wherein the synthetic fibers are at least partially encompassed by a liquid permeable material.

3.6.1.1. The article of paragraph 3.6.1, wherein the liquid permeable material is a tissue sheet.

3.7. The article of any of paragraphs 3-3.6.1.1, wherein the at least one component is retained on the synthetic fibers.

3.7.1. The article of paragraph 3.7, further comprising a binder, wherein the binder retains the at least one component on the synthetic fibers.

3.7.1.1. The article of paragraph 3.7.1, wherein the binder comprises one or more binders selected from the group consisting of ethyl cellulose, methyl cellulose, nitrocellulose, and polyurethane.

3.8. The article of any of paragraphs 3.7-3.7.1.1, further comprising a releasing agent adapted to facilitate the release of the at least one component from the synthetic fibers in the presence of an aqueous system.

3.9. An absorbent article incorporating the article of any of paragraphs 3-3.8.

4. An absorbent article, comprising:

a top sheet that is liquid permeable;

a back sheet that is liquid impermeable;

an absorbent material disposed between the top sheet and the back sheet;

at least one structural element selected from the group consisting of a liquid permeable tissue sheet, and a particle comprising hydrogen bonded cellulose pulp fibers; and

a chemiluminescent system adapted to react in the presence of an aqueous system to produce light;

wherein components of said chemiluminescent system are separately disposed in, or on, two or more of the group consisting of the absorbent material, the top sheet, and the at least one structural element, in a configuration in which a first component is transferred to a second component by the aqueous system moving through the absorbent article.

4.1. The absorbent article of paragraph 4, wherein a first of said components is disposed within the absorbent material.

4.1.1. The absorbent article of paragraph 4.1, further comprising a liquid permeable tissue sheet at least partially encompassing the absorbent material, wherein a second of said components is disposed on at least one surface of the tissue sheet.

4.1.1.1. The absorbent article of paragraph 4.1.1,

wherein the chemiluminescent system comprises a luciferin and a luciferase,

wherein the luciferase is disposed within the absorbent material, and

wherein the luciferin is disposed on the tissue sheet.

4.1.1.1.1. The absorbent article of paragraph 4.1.1.1,

wherein the luciferin is selected from the group consisting of coelenterazine, a coelenterazine analog, dinoflagellate luciferin, bacterial luciferin, fungal luciferin, firefly luciferin, vargulin, and furimazine; and

wherein the luciferase is selected from the group consisting of Gaussia luciferase, Renilla luciferase, Metridia luciferase, Oplophorus luciferase, dinoflagellate luciferase, copepod luciferase, and firefly luciferase.

4.1.1.1.2. The absorbent article of paragraph 4.1.1.1 or 4.1.1.1.1, wherein the luciferin is coelenterazine and the luciferase is one or more of Gaussia luciferase, Renilla luciferase, and Metridia luciferase.

4.1.2. The absorbent article of paragraph 4.1, wherein the absorbent material comprises fibers treated with said component.

4.1.2.1. The absorbent article of paragraph 4.1.2, wherein the treated fibers include cellulosic fibers.

5. An absorbent article, comprising:

a top sheet that is liquid permeable;

a back sheet that is liquid impermeable;

a fibrous, absorbent material comprising fibers treated with a luciferase; and

a tissue sheet, comprising at least one surface treated with a luciferin;

wherein the tissue sheet and the absorbent material are disposed between the top sheet and the back sheet in a configuration in which one of the luciferin and the luciferase is transferred to the other by an aqueous system moving through the absorbent article.

5.1. The absorbent article of paragraph 5, wherein the tissue sheet at least partially encompasses the absorbent material to form an absorbent core.

5.2. The absorbent article of either of paragraphs 5 or 5.1,

wherein the luciferin is selected from the group consisting of coelenterazine, a coelenterazine analog, dinoflagellate luciferin, bacterial luciferin, fungal luciferin, firefly luciferin, vargulin, and furimazine; and

wherein the luciferase is selected from the group consisting of Gaussia luciferase, Renilla luciferase, Metridia luciferase, Oplophorus luciferase, dinoflagellate luciferase, copepod luciferase, and firefly luciferase.

5.3. The absorbent article of any of paragraphs 5-5.2, wherein the luciferase is one or more of Gaussia luciferase, Renilla luciferase, Metridia luciferase, and wherein the luciferin is coelenterazine.

5.3.1. The absorbent article of paragraph 5.3, comprising 0.00001-100.0 mg coelenterazine and 0.00001-100.0 mg total luciferase.

5.3.2. The absorbent article of paragraph 5.3, comprising 0.0001-20.0 mg coelenterazine and 0.00003-20.0 mg total luciferase.

5.3.3. The absorbent article of paragraph 5.3, comprising 0.01-100.0 mg coelenterazine and 0.2-40 mg total luciferase.

6. A formulation, comprising:

at least one component of a chemiluminescent system that is adapted to react in the presence of an aqueous system to produce light, wherein the at least one component is selected from a luciferin and a luciferase; and

a liquid carrier.

6.1. The formulation of paragraph 6, wherein the at least one component is a luciferin.

6.1.1. The formulation of paragraph 6.1,

wherein the liquid carrier includes a solvent in which the luciferin is dissolved; and

wherein the formulation comprises 40-99 weight percent solvent and 0.01-20 weight percent luciferin.

6.1.1.1. The formulation of paragraph 6.1.1, wherein the solvent is selected from the group consisting of ethanol, isopropanol, n-butanol, isobutanol, ethyl acetate, methyl acetate, isopropyl acetate, acetone, pentanone, methyl ethyl ketone, n-butyl acetate, and combinations thereof.

6.1.1.2. The formulation of paragraph 6.1.1 or 6.1.1.1, wherein the solvent includes ethanol.

6.1.2. The formulation of paragraph 6.1 or 6.1.1, wherein the luciferin is selected from the group consisting of coelenterazine, a coelenterazine analog, dinoflagellate luciferin, bacterial luciferin, fungal luciferin, firefly luciferin, vargulin, and furimazine.

6.1.2.1. The formulation of any of paragraphs 6.1-6.1.2, wherein the luciferin is coelenterazine.

6.1.2.1.1. The formulation of paragraph 6.1.2.1, wherein the formulation comprises 0.1-0.3 weight percent coelenterazine.

6.1.2.1.2. The formulation of paragraph 6.1.2.1, wherein the formulation comprises 0.2-0.5 weight percent coelenterazine.

6.1.2.1.3. The formulation of paragraph 6.1.2.1, wherein the formulation comprises 0.5-0.9 weight percent coelenterazine.

6.1.2.1.4. The formulation of paragraph 6.1.2.1, wherein the formulation comprises 0.9-2.0 weight percent coelenterazine.

6.1.2.1.5. The formulation of paragraph 6.1.2.1, wherein the formulation comprises 2.0-10 weight percent coelenterazine.

6.1.2.1.6. The formulation of paragraph 6.1.2.1, wherein the formulation comprises 10-20 weight percent coelenterazine.

6.1.3. The formulation of any of paragraphs 6.1-6.1.2.1, wherein the liquid carrier is non-aqueous, and wherein the formulation further comprises 0.01-20 weight percent luciferase.

6.2. The formulation of any of paragraphs 6-6.1.3, further comprising a binder adapted to retain the at least one component on a substrate material to which the formulation is applied.

6.2.1. The formulation of paragraph 6.2, wherein the formulation comprises 0.1-30 weight percent of the binder.

6.2.2. The formulation of paragraph 6.2 or 6.2.1, wherein the binder is selected from the group consisting of ethyl cellulose, methyl cellulose, nitrocellulose, and polyurethane.

6.3. The formulation of any of paragraphs 6-6.1.3, further comprising a viscosity adjusting agent in an amount sufficient to impart a desired viscosity to the formulation.

6.3.1. The formulation of paragraph 6.3, wherein the formulation comprises 0.1-15 weight percent of the viscosity adjusting agent.

6.3.1.1. The formulation of paragraph 6.3 or 6.3.1, wherein the desired viscosity is a viscosity suitable for application of the formulation by streaming.

6.3.1.2. The formulation of paragraph 6.3 or 6.3.1, wherein the desired viscosity is a viscosity suitable for application of the formulation by printing.

6.3.1.3. The formulation of paragraph 6.3 or 6.3.1, wherein the viscosity is suitable for application of the formulation by coating.

6.3.2. The formulation of any of paragraphs 6.3-6.3.1.3, wherein the viscosity adjusting agent is selected from the group consisting of ethyl cellulose, methyl cellulose, nitrocellulose, and polyurethane.

6.4. The formulation of any of paragraphs 6.1-6.3.2, further comprising a porous transfer agent adapted, upon contact of a substrate material treated with the formulation with an aqueous system, to facilitate transfer of the component, and/or the aqueous system, relative to the substrate material.

6.4.1. The formulation of paragraph 6.4, comprising 0.01-52 weight percent porous transfer agent.

6.4.2. The formulation of paragraph 6.4 or 6.4.1, wherein the porous transfer agent is selected from the group consisting of starch, amorphous silica, clay minerals, cellulose pulp fiber, cotton fiber, and synthetic polymer fiber.

6.5. The formulation of paragraph 6, comprising both a luciferin and a luciferase.

6.5.1. The formulation of paragraph 6.5, wherein the liquid carrier includes a solvent in which the luciferin is dissolved, and wherein the luciferase is dispersed in the liquid carrier.

6.5.1.1 The formulation of paragraph 6.5.1, wherein the formulation comprises 40-99 weight percent solvent, 0.01-20 weight percent luciferin, and 0.01-20 weight percent luciferase.

6.5.1.1.1. The formulation of paragraph 6.5.1.1, wherein the luciferin comprises coelenterazine, and the luciferase comprises Gaussia luciferase, Renilla luciferase, and/or Metridia luciferase.

6.5.1.1.1.1. The formulation of paragraph 6.5.1.1.1, comprising 0.05 to 0.2 total weight percent luciferase.

6.5.1.1.1.2. The formulation of paragraph 6.5.1.1.1, comprising 0.2 to 0.6 total weight percent luciferase.

6.5.1.1.1.3. The formulation of paragraph 6.5.1.1.1, comprising 0.1 to 1.0 total weight percent luciferase.

6.5.1.1.1.4. The formulation of paragraph 6.5.1.1.1, comprising 1.0 to 10 total weight percent luciferase.

6.5.1.1.1.5. The formulation of paragraph 6.5.1.1.1, comprising 10 to 20 total weight percent luciferase.

6.5.2. The formulation of paragraph 6.5 or 6.5.1, wherein the luciferin comprises coelenterazine, the luciferase comprises Gaussia luciferase, Renilla luciferase, and/or Metridia luciferase, and the solvent comprises ethanol; and

wherein the formulation comprises:
50-99 weight percent ethanol;
0.1-5.0 weight percent coelenterazine;
0.1-5.0 total weight percent luciferase;
optionally, 0.01-30 total weight percent of one or more of:
   a binder adapted to retain the at least one component on a substrate material to which the formulation is applied; and
   a viscosity adjusting agent; and
optionally, 0.1-10 weight percent porous transfer agent.

6.5.3. The formulation of any of paragraphs 6.5-6.5.2, further including a binder, and wherein the binder is selected from the group consisting of ethyl cellulose, methyl cellulose, nitrocellulose, and polyurethane.

6.5.4. The formulation of any of paragraphs 6.5-6.5.3, further including a viscosity adjusting agent, and wherein the viscosity adjusting agent is selected from the group consisting of ethyl cellulose, methyl cellulose, nitrocellulose, and polyurethane.

6.5.5. The formulation of any of paragraphs 6.5-6.5.4, further including a porous transfer agent, and wherein the porous transfer agent is selected from the group consisting of starch, amorphous silica, clay minerals, cellulose pulp fiber, cotton fiber, and synthetic polymer fiber.

6.6. The formulation of paragraph 6, wherein the component is a luciferin, wherein the luciferin comprises coelenterazine, and wherein the liquid carrier includes a solvent in which the coelenterazine is dissolved and that comprises ethanol; and

wherein the formulation comprises:
40-90 weight percent ethanol;
0.1-5.0 weight percent coelenterazine;
0.01-15 weight percent binder; and
9-52 weight percent porous transfer agent.

6.6.1 The formulation of paragraph 6.6, further comprising a luciferase.

6.6.2. The formulation of paragraph 6.6 or 6.6.1, wherein the binder is selected from the group consisting of ethyl cellulose, methyl cellulose, nitrocellulose, and polyurethane.

6.6.3. The formulation of any of paragraphs 6.6-6.6.2, wherein the porous transfer agent is selected from the group consisting of starch, amorphous silica, clay minerals, cellulose pulp fiber, cotton fiber, and synthetic polymer fiber.

7. A partially aqueous formulation for applying a luciferin to a substrate material, the formulation comprising a luciferin, a solvent to dissolve the luciferin that comprises 40-99 weight percent water and 1-60 weight percent of an excipient adapted to facilitate the solubility of the luciferin in water, and, optionally, a binder adapted to bind luciferin to the substrate material.

7.1. The formulation of paragraph 7, wherein the excipient comprises a polar protic solvent other than water.

7.2. The formulation of paragraph 7 or 7.1, wherein the excipient is selected from the group consisting of hydroxypropyl-β-cyclodextrin, ethanol, butanol, propanol, isopropanol, pentanone, and combinations thereof.

7.3. The formulation of any of paragraphs 7-7.2, further including a binder, wherein the binder is selected from the group consisting of ethyl cellulose, methyl cellulose, and polyurethane, and wherein the formulation comprises up to 15 weight percent binder.

7.3.1. The formulation of paragraph 7.3, comprising 0.01 to 1.2 weight percent binder.

7.3.2. The formulation of paragraph 7.3, comprising 8.0 to 12.5 weight percent binder.

7.4. The formulation of any of paragraphs 7-7.3, further comprising a porous transfer agent.

7.4.1. The formulation of paragraph 7.4, wherein the porous transfer agent is selected from the group consisting of starch, amorphous silica, clay minerals, cellulose pulp fiber, cotton fiber, and synthetic polymer fiber.

7.5. The formulation of any of paragraphs 7-7.4.1, wherein the luciferin is selected from the group consisting of coelenterazine, a coelenterazine analog, dinoflagellate luciferin, bacterial luciferin, fungal luciferin, firefly luciferin, vargulin, and furimazine.

7.5.1. The formulation of paragraph 7.5, wherein the luciferin is coelenterazine.

7.5.1.1. The formulation of paragraph 7.5.1, wherein the excipient includes hydroxypropyl-β-cyclodextrin in a concentration of 45-50 mM, and wherein the formulation includes the luciferin in a concentration of up to 3.7 mM.

7.5.1.2. The formulation of paragraph 7, comprising up to 11 weight percent coelenterazine in solution.

8. A method of applying a luciferase to a substrate material capable of retaining up to a threshold level of moisture after any free water is removed, the method comprising:

applying a formulation that includes a luciferase dispersed in an aqueous liquid to a surface of the substrate material to achieve a luciferase concentration of the substrate of 0.01-20 mg per gram of substrate material;

wherein the applying step does not increase the moisture level of the substrate material above the threshold level of moisture.

8.1. The method of paragraph 8, wherein the substrate material is a fluff pulp sheet having a threshold level of moisture of at least 15 weight percent.

8.1.1. The method of paragraph 8.1, wherein the luciferase formulation is applied to the surface at a rate that increases the moisture level of the fluff pulp sheet by less than 10 weight percent.

8.2. The method of paragraph 8, wherein the substrate material has a threshold level of moisture of up to 25 weight percent.

8.3. The method of any of paragraphs 8-8.2, wherein the luciferase formulation has a luciferase concentration of 5.0-30 weight percent.

8.4. The method of any of paragraphs 8-8.3, wherein the luciferase is selected from the group consisting of Gaussia luciferase, Renilla luciferase, Metridia luciferase, Oplophorus luciferase, dinoflagellate luciferase, copepod luciferase, and firefly luciferase.

8.4.1. The method of paragraph 8.4, wherein the luciferase is one or more of Gaussia luciferase, Renilla luciferase, and Metridia luciferase.

9. A method of applying a chemiluminescent system, which reacts in the presence of free water to produce light, to a substrate material capable of retaining up to a threshold level of moisture after any free water is removed, the method comprising:

a luciferase treatment step, in which an area on a surface of the substrate is treated with a luciferase formulation that includes a luciferase in an aqueous liquid;

a luciferin treatment step, in which said area is treated with a luciferin formulation that includes a luciferin dissolved in a non-aqueous solvent;

wherein the luciferase treatment step does not increase the moisture content of the substrate material above the threshold level of moisture.

9.1. The method of paragraph 9, wherein the substrate is a fluff pulp sheet having a threshold level of moisture of at least 15 weight percent.

9.1.1. The method of paragraph 9.1, wherein the luciferase formulation is applied to the surface at a rate that increases the moisture level of the fluff pulp sheet by less than 10 weight percent.

9.2. The method of paragraph 9, wherein the substrate material has a threshold level of moisture of up to 25 weight percent.

9.3. The method of any of paragraphs 9-9.2, wherein the luciferase formulation has a luciferase concentration of 5.0-30 weight percent.

9.4. The method of any of paragraphs 9-9.3,
wherein the luciferin is selected from the group consisting of coelenterazine, a coelenterazine analog, dinoflagellate luciferin, bacterial luciferin, fungal luciferin, firefly luciferin, vargulin, and furimazine; and

wherein the luciferase is selected from the group consisting of Gaussia luciferase, Renilla luciferase, Metridia luciferase, Oplophorus luciferase, dinoflagellate luciferase, copepod luciferase, and firefly luciferase.

10. A method of producing a treated tissue composition for incorporation into an absorbent article, the method comprising:

applying a formulation to a surface of a liquid permeable tissue sheet, wherein the formulation comprises a luciferin and a solvent in which the luciferin is dissolved, and wherein the luciferin is retained on the tissue sheet upon applying the formulation; and

subsequently removing the solvent from the tissue sheet.

10.1. The method of paragraph 10, wherein applying includes streaming the formulation to the surface.

10.1.1. The method of paragraph 10.1,

wherein the formulation is streamed by means of a streaming apparatus, and

wherein applying further includes moving the tissue sheet relative to the streaming apparatus.

10.1.1.1. The method of paragraph 10.1.1, wherein the streaming apparatus includes one or more nozzles through which the formulation is streamed, with the one or more nozzles positioned to be in contact with the moving surface.

10.1.1.2. The method of paragraph 10.1.1 or 10.1.1.1, wherein the surface of the tissue sheet to which the formulation is streamed is suspended between two fixed points.

10.2. The method of any of paragraphs 10-10.1.2, wherein the tissue sheet is continuous.

10.3. The method of any of paragraphs 10-10.2, wherein removing the solvent includes heat treatment of the surface of the tissue sheet treated with the formulation.

10.4. The method of any of paragraphs 10-10.3, wherein the tissue sheet comprises cellulosic fibers, and wherein the formulation further includes a binder adapted to retain the luciferin on the cellulosic fibers.

10.4.1. The method of paragraph 10.4, wherein the tissue sheet further comprises synthetic fibers.

10.5. The method of any of paragraphs 10-10.4.1, wherein the formulation comprises 40-99 weight percent solvent and 0.01-20 weight percent luciferin.

10.5.1. The method of paragraph 10.5, wherein the luciferin is selected from the group consisting of coelenterazine, a coelenterazine analog, dinoflagellate luciferin, bacterial luciferin, fungal luciferin, firefly luciferin, vargulin, and furimazine.

10.5.1.1. The method of paragraph 10.5.1, wherein the luciferin is coelenterazine, and wherein the formulation is applied at a rate to produce a treated tissue composition having a coelenterazine concentration of 0.00002 to 20 weight percent.

10.5.2. The method of paragraph 10.5 or 10.5.1, wherein the formulation is applied at a rate to produce a treated tissue composition having a luciferin concentration of 0.001-10 weight percent.

10.5.3. The method of any of paragraphs 10.5-10.5.2, wherein the solvent includes ethanol.

10.5.4. The method of any of paragraphs 10.5-10.5.3, wherein the solvent comprises 40-99 weight percent water and 1-60 weight percent of an excipient configured to facilitate the solubility of the luciferin in water.

10.5.5. The method of any of paragraphs 10.5-10.5.4, wherein the formulation further comprises 0.01-30 weight percent of a binder adapted to retain the luciferin on the cellulosic fibers.

10.5.5.1 The method of paragraph 10.5.5, wherein the amount of binder is selected to impart a desired viscosity to the formulation.

10.6. The method of any of paragraphs 10-10.5.4.1, wherein the formulation is applied to the surface in the form of a longitudinal strip having a width that is less than or equal to the width of the tissue sheet.

10.6.1. The method of paragraph 10.6, wherein the longitudinal strip is continuous.

10.6.2. The method of paragraph 10.6, wherein the longitudinal strip is discontinuous.

10.6.2.1. The method of paragraph 10.6.2, wherein the longitudinal strip is one or more of dotted and dashed.

10.6.3. The method of any of paragraphs 10.6-10.6.2.1, wherein the longitudinal strip is straight.

10.6.4. The method of any of paragraphs 10.6-10.6.2.1, wherein the longitudinal strip includes one or more curved portions.

10.6.5. The method of any of paragraphs 10.6-10.6.2.1, wherein the longitudinal strip includes one or more straight portions.

10.7. The method of any of paragraphs 10-10.5.4.1, wherein the formulation is applied to the surface in the form of one or more shapes.

10.8. The method of any of paragraphs 10-10.7, wherein the formulation further comprises a luciferase.

10.9. The method of any of paragraphs 10-10.8, wherein the method is at least partially performed on a tissue printing machine.

10.10. The method of any of paragraphs 10-10.9, wherein the luciferin is coelenterazine, and wherein the formulation is applied at a rate to produce a treated tissue composition having 0.01 mg-25 g coelenterazine per 12″ length of tissue sheet.

10.10.1. The method of paragraph 10.10, wherein the formulation is applied at a rate to produce a treated tissue composition having 0.01-100 mg coelenterazine per 12″ length of tissue sheet.

10.10.2. The method of paragraph 10.10, wherein the formulation is applied at a rate to produce a treated tissue composition having 0.01-1.0 g coelenterazine per 12″ length of tissue sheet.

10.10.3. The method of any of paragraphs 10.10-10.10.2, wherein the tissue sheet to which the formulation is applied is continuous, and wherein the method further comprises partitioning the tissue sheet into discrete lengths subsequent to applying the formulation.

10.11. A method of producing an absorbent core for incorporation into an absorbent article, the method comprising:

producing a treated tissue composition according to the method of any of paragraphs 10-10.10;

at least partially encompassing an absorbent material with the treated composition.

11. A method of producing a liquid-impermeable back sheet structure treated with at least one component of a chemiluminescent system, which reacts in the presence of an aqueous system to produce light, for incorporation into an absorbent article, the method comprising:

applying a formulation to a surface of a liquid-impermeable back sheet, wherein the formulation comprises:
the at least one component, wherein the at least one component is selected from a luciferin and a luciferase;
a liquid carrier; and
a binder adapted to retain the component on the back sheet; and

subsequently removing at least some of the liquid carrier from the back sheet.

11.1. The method of paragraph 11, wherein the component is a luciferin, and wherein the liquid carrier includes a solvent in which the luciferin is dissolved and that comprises ethanol; and

wherein the formulation comprises:
40-90 weight percent ethanol;
0.1-5.0 weight percent luciferin;
0.01-15 weight percent binder; and
9-52 weight percent porous transfer agent.

11.2. The method of paragraph 11 or 11.1, wherein the luciferin is selected from the group consisting of coelenterazine, a coelenterazine analog, dinoflagellate luciferin, bacterial luciferin, fungal luciferin, firefly luciferin, vargulin, and furimazine.

11.2.1. The method of paragraph 11.2, wherein the luciferin comprises coelenterazine.

11.3. The method of any of paragraphs 11-11.2, wherein the binder is selected from the group consisting of ethyl cellulose, methyl cellulose, nitrocellulose, and polyurethane.

11.4. The method of any of paragraphs 11-11.3, wherein the porous transfer agent is selected from the group consisting of starch, amorphous silica, clay minerals, cellulose pulp fiber, cotton fiber, and synthetic polymer fiber.

11.5. The method of any of paragraphs 11-11.4, wherein the amount of one or more of the binder and the porous transfer agent is selected to impart a desired viscosity to the formulation.

11.6. The method of any of paragraphs 11-11.5, wherein applying includes streaming the formulation to the surface.

11.7. The method of any of paragraphs 11-11.6, further comprising applying a coating over the surface of the back sheet treated with the formulation, wherein the coating is one or more of water soluble or water permeable.

11.8. The method of any of paragraphs 11-11.7, wherein removing the liquid carrier includes heat treatment of the surface of the back sheet treated with the formulation.

11.9. The method of any of paragraphs 11-11.8, wherein removing the liquid carrier includes contacting the surface of the back sheet treated with the formulation with an absorbent material adapted to wick the liquid carrier from the surface of the back sheet.

11.9.1. The method of paragraph 11.9, wherein the at least one component in the formulation is a luciferin, and wherein the absorbent material is treated with a luciferase.

11.9.1.1. The method of paragraph 11.9.1, wherein the absorbent material is in the form of an absorbent core that includes one or more of absorbent fibers and superabsorbent polymer.

12. A composition, comprising:

an encapsulated material consisting of particles comprising a first component of a chemiluminescent system, wherein the particles each have coating that covers the entire surface of the particle, wherein the coating comprises a material that is one or more of water permeable and water soluble, and wherein the particles comprise a predetermined quantity of the component; and

a predetermined quantity of a second component of the chemiluminescent system;

wherein the predetermined quantities of the respective components are those sufficient to produce light of a desired intensity in the presence of an aqueous system.

12.1. An absorbent article, comprising:

a top sheet that is liquid permeable;

a back sheet that is liquid impermeable;

an absorbent material disposed between the top sheet and the back sheet; and

the composition of paragraph 12.

13. A method of producing an absorbent article for detection of an aqueous insult, the method comprising:

producing an encapsulated material consisting of particles comprising one of two reactive components of a chemiluminescent system, which is configured to produce light upon contact with an aqueous system, with a material that is one or more of water permeable and water soluble;

producing an absorbent article, including
disposing the encapsulated material between a top sheet that is liquid permeable and a back sheet that is liquid impermeable, and
disposing the other reactive component of the chemiluminescent system in a structural element of the absorbent article.

14. A method of producing a fluff pulp treated with a chemiluminescent system, the method comprising separately applying a non-aqueous solution that includes a luciferin, and an aqueous solution that includes a luciferase, to portions of a fluff pulp sheet.

14.1. The method of paragraph 14, wherein the respective portions to which the luciferin and the luciferase are applied are non-overlapping.

14.2. The method of paragraph 14 or 14.1, wherein the portions are on opposing surfaces of the fluff pulp sheet.

14.3. The method of paragraph 14 or 14.1, wherein the portions are on the same surface of the fluff pulp sheet.

15. A method of producing a fluff pulp composition, comprising:

fiberizing a sheet of fluff pulp fibers to produce a dispersion of individualized fluff pulp fibers in air; and

spraying a formulation of at least one component of a chemiluminescent system adapted to react in the presence of an aqueous system to produce light into the dispersion;

wherein the concentration of the at least one component retained on the individualized fluff pulp fibers is 0.0003-10.0 weight percent of the individualized fluff pulp fibers.

15.1. The method of paragraph 15,

wherein the fiberizing is performed in the chamber of a hammermill, and

wherein the formulation is sprayed into the chamber of the hammermill.

15.2. The method of paragraph 15,

wherein the fiberizing is performed in the chamber of a hammermill,

wherein the dispersion is air conveyed from the chamber, and

wherein the formulation is sprayed into the dispersion after it is air conveyed from the chamber.

16. A kit, comprising

an absorbent article, comprising a top sheet that is liquid permeable, a back sheet that is liquid impermeable, an absorbent material disposed between the top sheet and the back sheet, and a first component of a chemiluminescent system disposed between the top sheet and the back sheet, wherein the first component is one of a luciferin and a luciferase; and

a measured quantity of a second component of the chemiluminescent system, wherein the quantity is suitable for reaction with the first component, in the presence of an aqueous system, to produce light of a predetermined duration and/or intensity.

16.1 The kit of paragraph 16, wherein the second component is in the form of a liquid formulation, gel, or powder.

16.2. The kit of paragraph 16 or 16.1, wherein at least a portion of the absorbent article is configured to allow application of the second component thereto.

16.2.1. The kit of paragraph 16.2, wherein one or more of the top sheet and the back sheet are selectively openable and re-sealable.

17. A structural element for incorporation into an absorbent article, the structural element comprising:

a first surface having a treated area, the treated area being treated with at least one component of a chemiluminescent system adapted to react in the presence of an aqueous system to produce light;

wherein the at least one component is selected from a luciferin and a luciferase; and

wherein the total treated area is less than the area of the first surface.

17.1. The structural element of paragraph 17,

wherein the first surface includes a target region, which corresponds to a region in which one or more fluid insults are expected during use of an absorbent article into which the structural element is incorporated; and

wherein the treated area at least partially overlaps the target region.

17.1.1. The structural element of paragraph 17.1,

wherein the target region includes two or more regions that correspond to regions in which respective, sequential fluid insults are expected during use of the absorbent article; and

wherein the treated area includes two or more discrete portions which, respectively, at least partially overlap the two or more corresponding regions.

17.1.1.1. The structural element of paragraph 17.1.1, wherein the first portion of the treated area is configured to produce visible light that differs in at least one visual respect from that of the second portion.

17.1.1.1.1. The structural element of paragraph 17.1.1.1, wherein the first portion of the treated area is configured to provide visible light of different intensity with respect to that of the second portion.

17.1.1.1.2. The structural element of paragraph 17.1.1.1, wherein the first portion of the treated area is configured to provide visible light of different color with respect to that of the second portion.

17.1.1.1.3. The structural element of paragraph 17.1.1.1, wherein the first portion of the treated area is configured to provide visible light of different duration with respect to that of the second portion.

17.1.1.2. The structural element of any of paragraphs 17.1.1.-17.1.1.1.3, wherein each of the two or more discrete portions of the treated area only overlap the respective corresponding regions.

17.1.1.3. The structural element of any of paragraphs 17.1.1.-17.1.1.2, wherein the first and second portions of the treated area overlap the respective corresponding portions to a different extent.

17.1.1.4. The structural element of any of paragraphs 17.1.1.-17.1.1.3, wherein at least two of the corresponding regions are substantially concentric.

17.1.2. The structural element of paragraph 17.1,

wherein the target region includes two or more regions that correspond to regions in which respective, sequential fluid insults are expected during use of the absorbent article; and

wherein the treated area at least partially overlaps one, but not another, of the two or more corresponding regions.

17.2. The structural element of any of paragraphs 17-17.1.1.4, wherein the treated area is in the form of a continuous shape.

17.2.1. The structural element of paragraph 17.2, wherein the treated area is in the form of a longitudinal strip.

17.2.1.1. The structural element of paragraph 17.2 or 17.2.1, wherein the first surface has a length, and wherein the longitudinal strip has a length less than the length of the first surface.

17.3. The structural element of any of paragraphs 17-17.1.1.4, wherein the treated area is in the form of a pattern comprising two or more discrete portions.

17.3.1. The structural element of paragraph 17.3, wherein the pattern is in the form of a discontinuous, longitudinal strip.

17.3.1.1. The structural element of paragraph 17.3 or 17.3.1, wherein the first surface has a length, and wherein the longitudinal strip has a length less than the length of the first surface.

17.3.2. The structural element of paragraph 17.3, wherein the pattern is in the form of an arrangement of shapes.

17.3.3. The structural element of paragraph 17.3 or 17.3.2, wherein the pattern extends over an area less than the area of the first surface.

17.4. The structural element of any of paragraphs 17-17.3.1.1, wherein the at least one component is a luciferin.

17.4.1. The structural element of paragraph 17.4, wherein the luciferin is selected from the group consisting of coelenterazine, a coelenterazine analog, dinoflagellate luciferin, bacterial luciferin, fungal luciferin, firefly luciferin, vargulin, and furimazine.

17.5. The structural element of any of paragraphs 17-17.3.1.1, wherein the at least one component is a luciferase.

17.5.1. The structural element of paragraph 17.5, wherein the luciferase is selected from the group consisting of Gaussia luciferase, Renilla luciferase, Metridia luciferase, Oplophorus luciferase, dinoflagellate luciferase, copepod luciferase, and firefly luciferase.

17.6. The structural element of any of paragraphs 17-17.3.1.1, wherein the at least one component is a luciferin and a luciferase.

17.6.1. The structural element of paragraph 17.6, wherein the luciferin is selected from the group consisting of coelenterazine, a coelenterazine analog, dinoflagellate luciferin, bacterial luciferin, fungal luciferin, firefly luciferin, vargulin, and furimazine, and/or wherein the luciferase is selected from the group consisting of Gaussia luciferase, Renilla luciferase, Metridia luciferase, Oplophorus luciferase, dinoflagellate luciferase, copepod luciferase, and firefly luciferase.

17.6.2. The structural element of paragraph 17.6 or 17.6.1, wherein the treated area includes separate areas that are each treated with luciferin and luciferase.

17.6.2.1. The structural element of paragraph 17.6.2, wherein at least two separate areas are adjacent to each other.

17.7. The structural element of any of paragraphs 17-17.6.2.1, wherein the treated area is a first treated area, and wherein the first surface further comprises a second treated area treated with a non-chemiluminescent wetness indicator.

17.7.1. The structural element of paragraph 17.7, wherein the first and second treated areas are non-overlapping.

17.8. The structural element of any of paragraphs 17-17.7.1, wherein the treated area is 1-99% of the area of the surface, or any range therein.

17.9. The structural element of any of paragraphs 17-17.8, wherein the structural element is in a form selected from the group consisting of an absorbent core, a layer of material used in an absorbent core, a liquid permeable sheet, a liquid impermeable sheet, a tissue sheet, and a back sheet.

17.10. An absorbent article incorporating a structural element according to any of paragraphs 17-17.9, wherein the absorbent article is in a form selected from group consisting of an infant diaper, a wearable adult incontinence product, a feminine hygiene product, a pet pad, and a bed pad.

17.11. A method of producing a structural element according to any of paragraphs 17-17.9, the method comprising applying a formulation to the first surface of a structural element to form a treated area, wherein the formulation comprises the at least one component of the chemiluminescent system.

17.11.1. The method of paragraph 17.11, wherein the formulation is an ink formulation, and wherein applying the formulation includes applying the ink formulation by printing.

17.11.1.1 The method of paragraph 17.11.1, wherein the printing includes inkjet printing.

17.11.1.1.1. The method of paragraph 17.11.1.1, wherein the inkjet printing includes continuous inkjet printing.

18. A method of producing a structural element treated with at least one component of a chemiluminescent system, which reacts in the presence of an aqueous system to produce light, for incorporation into an absorbent article, the method comprising:

applying a formulation to a predetermined area of a first surface of a structural element, wherein the formulation comprises the at least one component, and wherein the at least one component is selected from a luciferin and a luciferase.

18.1. The method of paragraph 18, wherein the predetermined area at least partially overlaps a region in which one or more fluid insults are expected during use of an absorbent article into which the structural element is incorporated.

18.2. The method of paragraph 18 or 18.1, wherein the predetermined area is in the form of a continuous shape.

18.3. The method of any of paragraphs 18-18.2, wherein the predetermined area includes two or more discrete portions.

18.3.1. The method of paragraph 18.3, wherein the predetermined area is in the form of a pattern comprising two or more discrete portions.

18.4. The method of any of paragraphs 18-18.3.1, wherein the at least one component is a luciferin.

18.4.1. The method of paragraph 18.4, wherein the luciferin is selected from the group consisting of coelenterazine, a coelenterazine analog, dinoflagellate luciferin, bacterial luciferin, fungal luciferin, firefly luciferin, vargulin, and furimazine.

18.5. The method of any of paragraphs 18-18.3.1, wherein the at least one component is a luciferase.

18.5.1. The method of paragraph 18.5, wherein the luciferase is selected from the group consisting of Gaussia luciferase, Renilla luciferase, Metridia luciferase, Oplophorus luciferase, dinoflagellate luciferase, copepod luciferase, and firefly luciferase.

18.6. The method of any of paragraphs 18-18.3.1, wherein the at least one component is a luciferin and a luciferase.

18.6.1. The method of paragraph 18.6, wherein the luciferin is selected from the group consisting of coelenterazine, a coelenterazine analog, dinoflagellate luciferin, bacterial luciferin, fungal luciferin, firefly luciferin, vargulin, and furimazine, and/or wherein the luciferase is selected from the group consisting of Gaussia luciferase, Renilla luciferase, Metridia luciferase, Oplophorus luciferase, dinoflagellate luciferase, copepod luciferase, and firefly luciferase.

18.6.2. The method of paragraph 18.6, wherein applying a formulation includes applying at least two formulations, wherein one of the at least two formulations includes the luciferin, and wherein another of the at least two formulations includes the luciferase.

18.6.2.1. The method of paragraph 18.6.2, wherein applying a formulation includes separately applying the at least two formulations.

18.6.2.2. The method of paragraph 18.6.2. or 18.6.2.1, wherein the at least two formulations are applied, respectively, to non-overlapping portions of the predetermined area.

18.7. The method of any of paragraphs 18-18.6.2.2, wherein the formulation is an ink formulation, and wherein applying the formulation includes applying the ink formulation by printing.

18.7.1. The method of paragraph 18.7, wherein the printing includes inkjet printing.

18.7.1.1. The method of paragraph 18.7.1, wherein the inkjet printing includes continuous inkjet printing.

18.8. The method of any of paragraphs 18-18.7.1.1, further comprising applying a non-chemiluminescent wetness indicator to the first surface.

18.8.1. The method of paragraph 18.8, wherein the non-chemiluminescent wetness indicator is applied to at least partially overlap the predetermined area.

18.8.2. The method of paragraph 18.8, wherein the non-chemiluminescent wetness indicator is applied to not overlap the predetermined area.

1. A structural element for incorporation into an absorbent article, the structural element comprising:
a first surface having a treated area, the treated area being treated with at least one component of a chemiluminescent system adapted to react in the presence of an aqueous system to produce light;
wherein the at least one component is selected from a luciferin and a luciferase; and
wherein the treated area is less than an area of the first surface.

2. The structural element of claim 1,
wherein the first surface includes a target region that corresponds to a region in which one or more fluid insults are expected during use of an absorbent article into which the structural element is incorporated; and
wherein the treated area at least partially overlaps the target region.

3. The structural element of claim 2,
wherein the target region includes two or more regions that correspond to regions in which respective, sequential fluid insults are expected during use of the absorbent article; and
wherein the treated area includes two or more discrete portions which, respectively, at least partially overlap the two or more regions.

4. The structural element of claim 3, wherein a first portion of the two or more discrete portions is configured to produce visible light that differs in at least one visual respect from that of a second portion of the two or more discrete portions.

5-6. (canceled)

7. The structural element of claim 4, wherein the first portion of the treated area is configured to provide visible light of different duration with respect to that of the second portion.

8-10. (canceled)

11. The structural element of claim 2,
wherein the target region includes two or more regions that correspond to regions in which respective, sequential fluid insults are expected during use of the absorbent article; and
wherein the treated area at least partially overlaps one region, but not another region, of the two or more regions.

12. The structural element of claim 1, wherein the treated area is in the form of a continuous shape.

13. The structural element of claim 12, wherein the treated area is in the form of a longitudinal strip.

14. (canceled)

15. The structural element of claim 1, wherein the treated area is in the form of a pattern comprising two or more discrete portions.

16. The structural element of claim 15, wherein the pattern is in the form of a discontinuous, longitudinal strip.

17-19. (canceled)

20. The structural element of claim 1, wherein the at least one component is a luciferin.

21. The structural element of claim 20, wherein the luciferin is selected from the group consisting of coelenterazine, a coelenterazine analog, dinoflagellate luciferin, bacterial luciferin, fungal luciferin, firefly luciferin, vargulin, and furimazine.

22. The structural element of claim 1, wherein the at least one component is a luciferase.

23. The structural element of claim 22, wherein the luciferase is selected from the group consisting of Gaussia luciferase, Renilla luciferase, Metridia luciferase, Oplophorus luciferase, dinoflagellate luciferase, copepod luciferase, and firefly luciferase.

24. The structural element of claim 1, wherein the at least one component is a luciferin and a luciferase.

25. (canceled)

26. The structural element of claim 24, wherein the treated area includes separate areas wherein one area is each treated with luciferin and another area is treated with luciferase.

27-30. (canceled)

31. The structural element of claim 1, wherein the treated area is about 1-99% of the area of the first surface, or any range therein.

32. The structural element of claim 1, wherein the structural element is in a form selected from the group consisting of an absorbent core, a layer of material used in an absorbent core, a liquid-permeable sheet, a liquid-impermeable sheet, a tissue sheet, and a back sheet.

33. An absorbent article incorporating a structural element according to claim 1, wherein the absorbent article is in a form selected from group consisting of an infant diaper, a wearable adult incontinence product, a feminine hygiene product, a pet pad, and a bed pad.

34. A method of producing a structural element according to claim 1, the method comprising applying a formulation to the first surface of the structural element to form the treated area, wherein the formulation comprises the at least one component of the chemiluminescent system.

35. The method of claim 34, wherein the formulation is an ink formulation, and wherein applying the formulation includes applying the ink formulation by printing.

36-37. (canceled)

38. A method of producing a structural element treated with at least one component of a chemiluminescent system that reacts in the presence of an aqueous system to produce light for incorporation into an absorbent article, the method comprising:
applying a formulation to a predetermined area of a first surface of a structural element, wherein the formulation comprises the at least one component, and wherein the at least one component is selected from a luciferin and a luciferase.

39. The method of claim 38, wherein the predetermined area at least partially overlaps a region in which one or more fluid insults are expected during use of an absorbent article into which the structural element is incorporated.

40. The method of claim 38, wherein
the predetermined area is in the form of a continuous shape.

41. The method of claim 38, wherein the predetermined area includes two or more discrete portions.

42. (canceled)

43. The method of claim 38, wherein the at least one component is a luciferin.

44. The method of claim 43, wherein the luciferin is selected from the group consisting of coelenterazine, a coelenterazine analog, dinoflagellate luciferin, bacterial luciferin, fungal luciferin, firefly luciferin, vargulin, and furimazine.

45. The method of claim 38, wherein the at least one component is a luciferase.

46. The method of claim 45, wherein the luciferase is selected from the group consisting of Gaussia luciferase, Renilla luciferase, Metridia luciferase, Oplophorus luciferase, dinoflagellate luciferase, copepod luciferase, and firefly luciferase.

47. The method of claim 38, wherein the at least one component is a luciferin and a luciferase.

48. (canceled)

49. The method of claim 47, wherein applying a formulation includes applying at least two formulations, wherein one of the at least two formulations includes the luciferin, and wherein another of the at least two formulations includes the luciferase.

50. The method of claim 49, wherein applying the at least two formulations includes separately applying the at least two formulations.

51. (canceled)

52. The method of claim 38, wherein the formulation is an ink formulation, and wherein applying the formulation includes applying the ink formulation by printing.

53-57. (canceled)

58. A treated tissue composition comprising:
a liquid-permeable tissue sheet comprising cellulosic fibers and having two opposed surfaces;
wherein at least one surface of the two opposed surfaces is treated with at least one component of a chemiluminescent system, wherein the chemiluminescent system is adapted to react in the presence of an aqueous system to produce light;
wherein the at least one component is selected from a luciferin and a luciferase; and
wherein the at least one component is retained on the at least one surface.

59. The treated tissue composition of claim 58, wherein the at least one component is a luciferin.

60. The treated tissue composition of claim 59, wherein the luciferin is selected from the group consisting of coelenterazine, a coelenterazine analog, dinoflagellate luciferin, bacterial luciferin, fungal luciferin, firefly luciferin, vargulin, and furimazine.

61. The treated tissue composition of claim 59, wherein the luciferin is coelenterazine.

62. The treated tissue composition of claim 61, wherein the treated tissue composition comprises about 0.00002 to about 20 weight percent coelenterazine.

63. The treated tissue composition of claim 61, wherein the treated tissue composition comprises about 0.00002 to about 0.01 weight percent coelenterazine.

64. The treated tissue composition of claim 61, wherein the treated tissue composition comprises about 0.01 to about 2 weight percent coelenterazine.

65. The treated tissue composition of claim 61, wherein the treated tissue composition comprises about 2 to about 10 weight percent coelenterazine.

66. (canceled)

67. The treated tissue composition of claim 61, wherein the treated tissue composition comprises about 10 to about 20 weight percent coelenterazine.

68. The treated tissue composition of claim 61, wherein the liquid-permeable tissue sheet is about 0.1-235 mm wide, and comprises about 0.01 mg-25 g of coelenterazine per 12-inch length, with a weight of the coelenterazine per 12-inch length being less than or equal to a weight of a 12-inch length of untreated tissue sheet.

69. (canceled)

70. The treated tissue composition of claim 58, wherein the at least one component is retained on the at least one surface by a binder.

71. The treated tissue composition of claim 70, wherein the binder comprises one or more binders selected from the group consisting of ethyl cellulose, methyl cellulose, nitrocellulose, and polyurethane.

72-73. (canceled)

74. The treated tissue composition of claim 58, wherein a treated area of the at least one surface treated with the at least one component has a width less than or equal to a width of the liquid-permeable tissue sheet, and wherein the treated area is in the form of a longitudinal strip.

75-83. (canceled)

84. The treated tissue composition of claim 58, wherein the at least one component is a luciferase.

85. The treated tissue composition of claim 84, wherein the luciferase is selected from the group consisting of Gaussia luciferase, Renilla luciferase, Metridia luciferase, Oplophorus luciferase, dinoflagellate luciferase, copepod luciferase, and firefly luciferase.

86. The treated tissue composition of claim 58, wherein the at least one component is a luciferin and a luciferase.

87. (canceled)

88. The treated tissue composition of claim 86, wherein the luciferin is coelenterazine and the luciferase is selected from the group consisting of Gaussia luciferase, Renilla luciferase, and Metridia luciferase.

89. The treated tissue composition of claim 58, further comprising a releasing agent adapted to facilitate release of the at least one component from the at least one surface in the presence of an aqueous system.

90. The treated tissue composition of claim 58, comprising at least two liquid-permeable layers, one of which is the liquid-permeable tissue sheet.

91. The treated tissue composition of claim 90, comprising a first and a second liquid-permeable tissue sheet, wherein at least one surface of each tissue sheet is treated with a different component of the chemiluminescent system.

92. (canceled)

93. An absorbent core for an absorbent article comprising the treated tissue composition of claim 58.

94. The absorbent core of claim 93, comprising an absorbent structure that is at least partially encompassed by the treated tissue composition of claim 58.

95. (canceled)

96. An absorbent article comprising the absorbent core of claim 93.

97. An absorbent core for an absorbent article, comprising an absorbent structure that is at least partially encompassed by the treated tissue composition of claim 58, wherein the treated tissue composition comprises luciferin, and wherein the absorbent structure comprises luciferase.

98-247. (canceled)

248. A method of producing a treated tissue composition for incorporation into an absorbent article, the method comprising:
applying a formulation to a surface of a liquid-permeable tissue sheet, wherein the formulation comprises a luciferin and a solvent in which the luciferin is dissolved, and wherein the luciferin is retained on the liquid-permeable tissue sheet upon applying the formulation; and
subsequently removing the solvent from the liquid-permeable tissue sheet.

249. The method of claim 248, wherein applying includes streaming the formulation to the surface.

250. The method of claim 249,
wherein the formulation is streamed with a streaming apparatus, and
wherein applying further includes moving the liquid-permeable tissue sheet relative to the streaming apparatus.

251. The method of claim 250, wherein the streaming apparatus includes one or more nozzles through which the formulation is streamed, wherein the one or more nozzles are positioned to be in contact with the surface.

252. (canceled)

253. The method of claim 248, wherein the liquid-permeable tissue sheet is a continuous sheet.

254. The method of claim 248, wherein removing the solvent comprises heating the surface of the liquid-permeable tissue sheet treated with the formulation.

255. The method of claim 248, wherein the liquid-permeable tissue sheet comprises cellulosic fibers, and wherein the formulation further includes a binder adapted to retain the luciferin on the cellulosic fibers.

256. (canceled)

257. The method of claim 248, wherein the formulation comprises about 40-99 weight percent solvent and about 0.01-20 weight percent luciferin.

258. The method of claim 257, wherein the luciferin is selected from the group consisting of coelenterazine, a coelenterazine analog, dinoflagellate luciferin, bacterial luciferin, fungal luciferin, firefly luciferin, vargulin, and furimazine.

259. The method of claim 258, wherein the luciferin is coelenterazine, and wherein the formulation is applied at a rate to produce the treated tissue composition having a coelenterazine concentration of about 0.00002 to about 20 weight percent.

260. The method of claim 257, wherein the formulation is applied at a rate to produce the treated tissue composition having a luciferin concentration of about 0.001-10 weight percent.

261. The method of claim 257, wherein the solvent includes ethanol.

262. The method of claim 257, wherein the solvent comprises about 40-99 weight percent water and about 1-60 weight percent of an excipient configured to facilitate the solubility of the luciferin in water.

263. The method of claim 257, wherein the formulation further comprises about 0.01-30 weight percent of a binder adapted to retain the luciferin on the cellulosic fibers.

264. The method of claim 263, wherein an amount of the binder is selected to impart a desired viscosity to the formulation.

265. The method of claim 248, wherein the formulation is applied to the surface in the form of a longitudinal strip having a width that is less than or equal to the width of the tissue sheet.

266. The method of claim 265, wherein the longitudinal strip is a continuous longitudinal strip.

267-272. (canceled)

273. The method of claim 248, wherein
the formulation further comprises a luciferase.

274. The method of claim 248, wherein
the method is at least partially performed on a tissue printing machine.

275. The method of claim 248, wherein the luciferin is coelenterazine, and wherein the formulation is applied at a rate to produce the treated tissue composition having about 0.01 mg-25 g coelenterazine per 12″ length of tissue sheet.

276. The method of claim 275, wherein the formulation is applied at a rate to produce the treated tissue composition having about 0.01-100 mg coelenterazine per 12″ length of tissue sheet.

277. The method of claim 248, wherein the formulation is applied at a rate to produce the treated tissue composition having about 0.01-1.0 g coelenterazine per 12″ length of tissue sheet.

278. The method of claim 275, wherein the liquid-permeable tissue sheet to which the formulation is applied is continuous, and wherein the method further comprises partitioning the liquid-permeable tissue sheet into discrete lengths subsequent to applying the formulation.

279-307. (canceled)
